(19) United States
(12) Patent Application Publication
(10) Pub. No.: US 2011/0117055 A1
(43) Pub. Date: May 19, 2011

(54) Methods of Treating Hepatitis C Virus with Oxoacetamide Compounds

(76) Inventors: James E. MacDonald, Encinitas, CA (US); McKelvy F. Jeffrey, San Diego, CA (US); Flossie Wong-Staal, San Diego, CA (US)

(21) Appl. No.: 12/949,646

(22) Filed: Nov. 18, 2010

Related U.S. Application Data
(60) Provisional application No. 61/262,899, filed on Nov. 19, 2009.

(51) Int. Cl. A61K 38/21; A61P 31/14; A61K 31/5375; A61K 31/167; A61K 31/18; A61K 31/4418; C07D 295/088; C07C 311/08; C07C 235/84; C07D 413/12; A61K 31/5377; C07C 235/78; C07D 417/12; A61K 31/541; A61K 31/553

(52) U.S. Cl. 424/85.4; 514/239.2; 514/616; 514/605; 514/621; 514/352; 544/165; 564/99; 564/158; 544/131; 514/235.5; 544/146; 514/231.5; 564/169; 544/141; 544/86; 514/231.8; 544/58.2; 514/227.8; 540/544; 514/211.15

(57) ABSTRACT

Provided herein are hepatitis C virus entry inhibitor oxoacetamide compounds, pharmaceutical compositions thereof, and methods for their use in treatment or prevention of hepatitis C virus infection in a subject in need thereof.

This application claims the benefit of U.S. Provisional Patent Application No. 61/262,899, filed Nov. 19, 2009, the entirety of which is incorporated herein by reference.

### FIELD

Provided herein are hepatitis C virus entry inhibitor oxoacetamide compounds, pharmaceutical compositions thereof, and methods for their use in treatment or prevention of hepatitis C virus infection in a subject in need thereof.

### BACKGROUND

In 1989, a main causative virus of non-A non-B post-transfusion hepatitis was found and named hepatitis C virus (HCV). Since then, several types of hepatitis viruses have been found besides type A, type B and type C, wherein hepatitis caused by HCV is called hepatitis C. Subjects infected with HCV are considered to involve several percent of the world population, and infection with HCV characteristically becomes chronic.

HCV is an enveloped RNA virus, wherein the genome is a single strand plus-strand RNA, and belongs to the genus Hepacivirus of Flavivirus (from The International Committee on Taxonomy of Viruses, International Union of Microbiological Societies). Of the same hepatitis viruses, for example, hepatitis B virus (HBV), which is a DNA virus, is eliminated by the immune system, and infection with this virus ends in an acute infection except for neonates and infants having yet immature immunological competence. In contrast, HCV somehow avoids the immune system of the host due to an unknown mechanism. Once infected with this virus, even an adult having a mature immune system frequently develops persistent infection.

When chronic hepatitis is associated with the persistent infection with HCV, it advances to cirrhosis or hepatic cancer in a high rate. Enucleation of tumor by operation does not help much, because the subject often develops recurrent hepatic cancer due to the sequela inflammation in non-cancerous parts.

Thus, an effective therapeutic method for treating hepatitis C infection is desired. Apart from the symptomatic therapy to suppress inflammation with an anti-inflammatory agent, the development of a therapeutic agent that reduces HCV to a low level free from inflammation and that eradicates HCV has been strongly demanded. An optimal therapeutic agent would provide a virologic response classified as a “sustained virologic response,” which is defined as undetectable levels of virus in blood six months or more after completing hepatitis C therapy.

Currently, an effective HCV vaccine has not been found. The only effective method known for the eradication of HCV at present is treatment with an interferon (e.g., Pegasys or Pegintron®), as a single agent or in combination with the nucleoside analog ribavirin. However, even the most effective HCV therapy, alpha-interferon and ribavirin, leads to sustained efficacy in only about 40% of patients (Poynard et al., Lancet 1998, 352, 1426-1432). For the rest of the subjects, it has no effect or provides only a temporary effect. Therefore, an anti-HCV drug to be used in the place of or concurrently with interferon and/or ribavirin is greatly needed.

Various therapeutic stratagems for HCV are undergoing clinical and pre-clinical testing, including the inhibition of protein processing or virus RNA replication. See, e.g., Hugle et al., C. Rev. Med. Virol., 2003, 13: 361-371. One such strategy, which is general for the development of antiviral agents, is to inactivate virally encoded enzymes that are essential for the replication of the virus. For example, NS3 protease inhibitors have been studied for the potential treatment of HCV infections via viral replication mechanisms. See Tan, et al., Nature Rev. Drug Discov., 2002, 1: 867-881. However, many of these agents lose therapeutic effectiveness due to drug resistance resulting from the high rate of mutation in HCV. See, e.g., Zein et al., Clin. Microbiol. Rev., 2000, 13: 223-235. Thus, while agents that target viral replication are potentially promising as HCV therapeutics, the development of agents which attach the virus via alternative mechanisms will remain a pressing issue for the foreseeable future.

An emerging area of antiviral research is the area of small molecule entry inhibitors. These drugs are designed to block the entry of a virus into a mammalian cell by interfering with various phases of attachment and/or fusion between the virus and the cell. For example, two HIV entry inhibitors, enfuvirtide (Fuzeon®) and maraviroc (Selzentry®), exist as marketed drugs while many others are under development. See, e.g., Reeves, J. D., ENTRY INHIBITORS IN HIV THERAPY (Reeves, J. D., Derdeyn, C. A., ed., Birkhauser 2007); Liu, S. et al., J. Biol. Chem., 2005, 280: 11259-11273.

The development of HCV entry inhibitors would be advantageous over current therapy for many reasons. HCV entry inhibitors may be used to compliment the current standard of treatment for HCV, interferon and ribavirin, as these drugs work by alternative mechanisms. HCV entry inhibitors are likely to be more effective in treating drug-resistant strains of HCV. Furthermore, the use of an HCV entry inhibitor is less likely to lead to drug resistance as the entry inhibitor targets the host cell instead of the virus itself.

In light of the fact that HCV infection has reached epidemic levels worldwide, new effective compositions, including for example HCV entry inhibitors, for the treatment or prevention of hepatitis C infection are needed.

### SUMMARY

Provided herein are hepatitis C virus entry inhibitor compounds, pharmaceutical compositions thereof, and methods for their use in treatment or prevention of hepatitis C virus infection in a subject in need thereof. Without being limited to a particular mechanism of action, compounds provided herein are capable of inhibiting the entry of HCV into a host cell by interacting with SR-B1, the host hepatocyte cell membrane protein involved in the docking and entry of HCV into the host. Thus, provided herein are HCV entry inhibitors and pharmaceutical compositions thereof, for use in treatment or prevention of HCV infection in a subject in need thereof.

In certain embodiments, the HCV entry inhibitor is selected from the group consisting of oxoacetamide compounds of general formula (I) or (II):

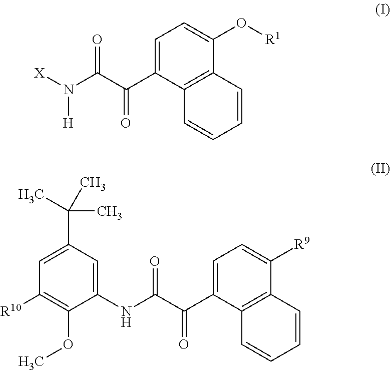

wherein

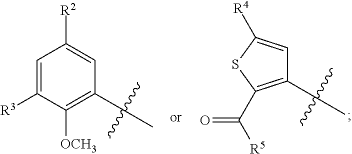

X is

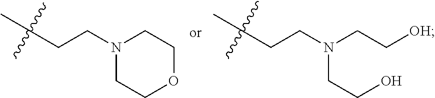

R¹ is methyl, ethyl, isopropyl,
R² is C₁-C₈ alkyl; C₅-C₈ cycloalkyl, or C₇-C₁₀ arylalkyl;
R³ is hydrogen, cyano, —CONHR⁶, —NHSO₂R⁷ or —SO₂N(R⁸)₂;
R⁴ is C₁-C₄ alkyl;
R⁵ is C₁-C₄ alkoxy or —N(R⁸)₂;
R⁶ is 2-pyridyl or C₁-C₆ alkyl, wherein one or more carbon atoms is optionally replaced by an oxygen atom;
R⁷ is C₁-C₄ alkyl, CH₂CF₃, benzyl or phenyl; and
R⁸ is C₁-C₄ alkyl;
R⁹ is bromo or 6-(methylamino)pyridin-3-yl;
R¹⁰ is hydrogen or —CONHR¹¹; and
R¹¹ is hydrogen or C₁-C₄ alkyl;
provided that if R³ is —NHSO₂R⁷ and R⁷ is methyl, then R¹ is not methyl;
provided that if R¹⁰ is hydrogen, R⁹ is 6-(methylamino)pyridin-3-yl; and
provided that if R¹¹ is cyclopropyl, R⁹ is bromo.

It is to be understood that the compounds provided herein may contain chiral centers. Such chiral centers may be of either the (R) or (S) configuration, or may be a mixture thereof. In certain cases the substituents of the compounds of formula I may contribute to optical and/or stereoisomerism. Thus, the compounds provided herein may be enantiomerically pure, or be stereoisomeric or diastereomeric mixtures.

Also provided herein are pharmaceutically acceptable salts of the compounds of formula I.

In one embodiment, the compounds provided herein are present in a substantially pure form. As used herein, “substantially pure” means sufficiently homogeneous to appear free of readily detectable impurities as determined by standard methods of analysis, such as thin layer chromatography (TLC), gel electrophoresis, high performance liquid chromatography (HPLC), nuclear magnetic resonance (NMR), and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. The instant disclosure is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

Also provided herein are isotopically enriched analogs of the compounds provided herein. Isotopic enrichment (for example, deuteration) of pharmaceuticals to improve pharmacokinetics (“PK”), pharmacodynamics (“PD”), and toxicity profiles, has been demonstrated previously with some classes of drugs. See, for example, Lijinsky et. al., Food Cosmet. Toxicol., 20: 393 (1982); Lijinsky et. al., J. Nat. Cancer Inst., 69: 1127 (1982); Mangold et. al., Mutation Res., 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D., Chem. Biol. Interact., 117: 191 (1999).

Isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). See, e.g, Foster et al., Adv. Drug Res., 1985, 14: 1-36; Kushner et al., Can. J. Physiol. Pharmacol., 1999, 77: 79-88.

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, will provide a similar kinetic isotope effects.

For example, the DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching. The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

Therefore, isotopic enrichment at certain positions of a compound provided herein will produce a detectable KIE that will affect the pharmacokinetic, pharmacologic, and/or toxicological profiles of a compound provided herein in comparison with a similar compound having a natural isotopic composition.

### BRIEF DESCRIPTION OF THE DRAWINGS

FIG. 1 is a schematic structure of HCV2aChLuc (HCV2aJ6/JFH Chimeric Renilla-Luciferase clone) genome.

FIG. 2 shows the characterization of HCV2aChLuc viral infection in cell culture.

FIG. 3 is a graphical depiction of the dose-dependent inhibitory effect of Compound 1 and Compound 5 on HCV2aChLuc viral entry.

FIG. 4 demonstrates that Compound 1 and Compound 5 did not measurably inhibit HCV RNA replication.

FIG. 5 demonstrates that Compound 1 and Compound 5 were not measurably toxic to Huh7 cells.

FIG. 6 demonstrates that Compound 1 has no measurable inhibitory effect on BVDV infection.

FIG. 7 is a schematic structure of HCV1a/2a chimeras with adaptive mutations.

FIG. 8 demonstrates that Compound 1 and Compound 5 each inhibit both HCV bearing structural proteins of genotype 1a or 2a.

FIG. 9 is a graphical depiction of the effects of the combination of Compound 1 or Compound 5 with IFN-α (interferon-alpha).

FIG. 10 is a graphical depiction of the effects of the combination of Compound 1 or Compound 5 with ribavirin.

FIG. 11 is a graphical depiction of the effects of the combination of Compound 1 or Compound 5 with HCV NS3 protease inhibitor VX950.

FIG. 12 demonstrates that HCV2aChLuc (NS3:A156S) mutant is highly resistant to VX950 but not Compounds 1 and 5.

FIG. 13 is (a) a schematic structure of the E2:G451R mutation introduced into the backbone of HCV2aCh, and (b) a graphical depiction demonstrating that the E2:G451R mutation had a reduced dependency on SR-B1 and increased binding to CD81.

FIG. 14 shows the antiviral activity of Compound 1 with the HCV2aCh (E2:G451R) mutant virus.

FIG. 15 shows the antiviral activity of Compound 5 with the HCV2aCh (E2:G451R) mutant virus.

FIG. 16 shows immuno-fluorescence read-outs demonstrating the antiviral activity of Compound 5 with the HCV2aCh (E2:G451R) mutant virus.

FIG. 17 is a graphical depiction of the synergistic effects of the combination of Compound 1 or Compound 5 with HCV NS3 protease inhibitor VX950.

FIG. 18 shows the Combination Index (CI) of Compound 5 in combination with various anti-HCV compounds.

FIG. 19 shows the Combination Index (CI) of Compound 5 in combination with VX-950 and relative luciferase activities of the compounds.

FIG. 20 shows the relative luciferase activities of Compound 5 and ribavirin, both alone and in combination.

FIG. 21 shows that a HCV2aChRLuc (A156S) protease mutant is resistant to VX-950 but not to Compound 5.

### DETAILED DESCRIPTION

Provided herein are methods of treating or preventing hepatitis C infection in a subject in need thereof, and pharmaceutical compositions and dosage forms useful for such methods. The methods and compositions are described in detail in the sections below.

### DEFINITIONS

When referring to the compounds provided herein, the following terms have the following meanings unless indicated otherwise. Unless defined otherwise, all technical and scientific terms used herein have the same meaning as is commonly understood by one of ordinary skill in the art. In the event that there are a plurality of definitions for a term herein, those in this section prevail unless stated otherwise.

“Pharmaceutically acceptable salt” includes any salt of a compound provided herein which retains its biological properties and which is not toxic or otherwise undesirable for pharmaceutical use. Such salts may be derived from a variety of organic and inorganic counter-ions well known in the art. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Salts further include, by way of example only, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

The term “host”, as used herein, includes any unicellular or multicellular organism in which the virus can replicate, including cell lines and animals, and preferably a human. Alternatively, the host can be carrying a part of the Flaviviridae viral genome, whose replication or function can be altered by the compounds provided herein. The term host specifically includes infected cells, cells transfected with all or part of the Flaviviridae genome and animals, in particular, primates (including chimpanzees) and humans. In most animal applications, the host is a human patient. Veterinary applications, in certain indications, however, are clearly anticipated herein (such as chimpanzees).

As used herein, the terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In one embodiment, the subject is refractory or non-responsive to current treatments for hepatitis C infection. In another embodiment, the subject is a farm animal (e.g., a horse, a cow, a pig, etc.) or a pet (e.g., a dog or a cat). In one embodiment, the subject is a human.

As used herein, the IC₅₀ refers to an amount, concentration or dosage of a particular test compound that achieves a 50% inhibition of a maximal response in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In one embodiment, a therapeutic agent is an agent which is known to be useful for, or has been or is currently being used for the treatment or prevention of a disorder or one or more symptoms thereof.

As used herein, the terms “subject” and “patient” are used interchangeably herein. The terms “subject” and “subjects” refer to an animal, preferably a mammal including a non-primate (e.g., a cow, pig, horse, cat, dog, rat, and mouse) and a primate (e.g., a monkey such as a cynomolgous monkey, a chimpanzee and a human), and more preferably a human. In one embodiment, the subject is refractory or non-responsive to current treatments for hepatitis C infection. In another embodiment, the subject is a farm animal (e.g., a horse, a cow, a pig, etc.) or a pet (e.g., a dog or a cat). In a preferred embodiment, the subject is a human.

As used herein, the terms “therapeutic agent” and “therapeutic agents” refer to any agent(s) which can be used in the treatment, management, or amelioration of a disorder or one or more symptoms thereof. In certain embodiments, the term “therapeutic agent” refers to a compound provided herein. In certain other embodiments, the term “therapeutic agent” refers does not refer to a compound provided herein. Preferably, a therapeutic agent is an agent which is known to be useful for, or has been or is currently being used for the treatment, management, prevention, or amelioration of a disorder or one or more symptoms thereof.

As used herein, “therapeutically effective amount” means an amount of a compound or complex or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

As used herein, “treating” or “treatment” of any disease or disorder refers, in one embodiment, to ameliorating a disease or disorder that exists in a subject. In another embodiment, “treating” or “treatment” refers to ameliorating at least one physical parameter, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

As used herein, the terms “prophylactic agent” and “prophylactic agents” as used refer to any agent(s) which can be used in the prevention of a disorder or one or more symptoms thereof. In certain embodiments, the term “prophylactic agent” refers to a compound provided herein. In certain other embodiments, the term “prophylactic agent” does not refer a compound provided herein. Preferably, a prophylactic agent is an agent which is known to be useful for, or has been or is currently being used to the prevent or impede the onset, development, progression and/or severity of a disorder.

As used herein, the terms “prevent,” “preventing” and “prevention” refer to the prevention of the recurrence, onset, or development of one or more symptoms of a disorder in a subject resulting from the administration of a therapy (e.g., a prophylactic or therapeutic agent), or the administration of a combination of therapies (e.g., a combination of prophylactic or therapeutic agents).

As used herein, the phrase “prophylactically effective amount” refers to the amount of a therapy (e.g., prophylactic agent) which is sufficient to result in the prevention of the development, recurrence or onset of one or more symptoms associated with a disorder (, or to enhance or improve the prophylactic effect(s) of another therapy (e.g., another prophylactic agent).

As used herein, “isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen”, the position is understood to have hydrogen at its natural isotopic composition.

As used herein, “isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

As used herein, the nomenclature alkyl, alkoxy, carbonyl, etc. is used as is generally understood by those of skill in this art.

As used herein, “alkyl” carbon chains, if not specified, contain from 1 to 20 carbons, 1 to 16 carbons or 1 to 6 carbons and are straight, branched or cyclic. Alkyl groups that are cyclic include cycloalkyl carbon chains as defined herein, or alkyl carbon chains in which part of the chain is cyclic, e.g., methylenecyclopropane, methylenecyclobutane, etc. In certain embodiments, alkyl carbon chains contain from 1 to 6 carbons. Exemplary alkyl groups herein include, but are not limited to, methyl, ethyl, propyl, isopropyl, isobutyl, n-butyl, sec-butyl, tert-butyl, isopentyl, neopentyl, tert-pentyl, isohexyl. As used herein, lower alkyl refers to carbon chains having from about 1 carbons up to about 6 carbons.

As used herein, “alkenyl” carbon chains, if not specified, contain from 2 to 20 carbons, 2 to 16 carbons or 2 to 6 carbons and are straight or branched. In certain embodiments, alkenyl carbon chains contain from 2 to 6 carbons. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. The alkenyl carbon chains of 2 to 6 carbons, in certain embodiments, contain 1 to 2 double bonds. Exemplary alkenyl groups herein include, but are not limited to, vinyl, 1-propenyl, 2-propenyl, isopropenyl, 1-butenyl, 2-butenyl, 3-butenyl, 1,3-butadienyl. As used herein, lower alkenyl refer to carbon chains having from about 2 carbons up to about 6 carbons.

As used herein, “alkynyl” carbon chains, if not specified, contain from 2 to 20 carbons, 2 to 16 carbons or 2 to 6 carbons and are straight or branched. In certain embodiments, alkynyl carbon chains contain from 2 to 6 carbons. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Alkynyl carbon chains of from 2 to 6 carbons, in certain embodiments, contain 1 to 2 triple bonds. Exemplary alkynyl groups herein include, but are not limited to, ethynyl, 1-propynyl and 2-propynyl. As used herein, lower alkynyl refer to carbon chains having from about 2 carbons up to about 6 carbons.

As used herein, “aryl” refers to aromatic monocyclic or multicyclic groups containing from 6 to 19 carbon atoms. Aryl groups include, but are not limited to groups such as fluorenyl, substituted fluorenyl, phenyl, substituted phenyl, naphthyl and substituted naphthyl.

As used herein, “cycloalkyl” refers to a saturated mono- or multicyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spino-connected fashion.

As used herein, “halo”, “halogen” or “halide” refers to F, Cl, Br or I.

As used herein, “haloalkyl” refers to an alkyl group in which one or more of the hydrogen atoms are replaced by halogen. “Lower haloalkyl” refers to a lower alkyl group in which one or more of the hydrogen atoms are replaced by halogen. Such groups include, but are not limited to, chloromethyl, trifluoromethyl and 1-chloro-2-fluoroethyl.

As used herein, “arylalkyl” refers to an aryl group which is bonded to an alkyl group. The point of attachment of the arylalkyl group may though either the aryl or the alkyl moiety. Such groups include, but are not limited to, benzyl (i.e., phenylmethyl), phenylethyl and 1-methyl-1-phenylethyl.

As used herein, “acyl” refers to a radical —C(O)R, where R is hydrogen, alkyl, cycloalkyl, cycloheteroalkyl, aryl, arylalkyl, heteroalkyl, heteroaryl, heteroarylalkyl as defined herein. Representative examples include, but are not limited to, formyl, acetyl, cylcohexylcarbonyl, cyclohexylmethylcarbonyl, benzoyl, benzylcarbonyl and the like.

As used herein, “alkoxy” refers to the group —OR where R is alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

As used herein, “alkoxycarbonyl” refers to a radical —C(O)-alkoxy where alkoxy is as defined herein.

As used herein, “amino” refers to the radical —NH₂.

As used herein, “alkylamino” refers to the group alkyl-NR′—, wherein R′ is selected from hydrogen and alkyl.

As used herein, “dialkylamino” means a radical —NRR′ where R and R′ independently represent an alkyl, substituted alkyl, aryl, substituted aryl, cycloalkyl, substituted cycloalkyl, cycloheteroalkyl, substituted cycloheteroalkyl, heteroaryl, or substituted heteroaryl group as defined herein.

As used herein, “carboxy” refers to the radical —C(O)OH.

As used herein, “hydroxy” refers to the radical —OH.

As used herein, “nitro” refers to the radical —NO₂.

As used herein, “cyano” refers to the radical —CN.

As used herein, “solvate” refers to a compound provided herein or a salt thereof, that further includes a stoichiometric or non-stoichiometric amount of solvent bound by non-covalent intermolecular forces. Where the solvent is water, the solvate is a hydrate.

It is to be understood that compounds having the same molecular formula but differing in the nature or sequence of bonding of their atoms or in the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers”. When a compound has an asymmetric center, for example, when it is bonded to four different groups, a pair of enantiomers is possible. An enantiomer can be characterized by the absolute configuration of its asymmetric center and is designated (R) or (S) according to the rules of Cahn and Prelog (Calm et al., 1966, Angew. Chem. 78:413-447, Angew. Chem., Int. Ed. Engl. 5:385-414 (errata: Angew. Chem., Int. Ed. Engl. 5:511); Prelog and Helmchen, 1982, Angew. Chem. 94:614-631, Angew. Chem. Internat. Ed. Eng. 21:567-583; Mata and Lobo, 1993, Tetrahedron:Asymmetry 4:657-668) or can be characterized by the manner in which the molecule rotates the plane of polarized light and is designated dextrorotatory or levorotatory (i.e., as (+)- or (−)-isomers, respectively). A chiral compound can exist as either individual enantiomer or as a mixture thereof. A mixture containing equal proportions of enantiomers is called a “racemic mixture.”

In certain embodiments, the compounds provided herein may possess one or more asymmetric centers; such compounds can therefore be produced as the individual (R)- or (S)-enantiomer or as a mixture thereof. Unless indicated otherwise, for example by designation of stereochemistry at any position of a formula, the description or naming of a particular compound in the specification and claims is intended to include both individual enantiomers and mixtures, racemic or otherwise, thereof. Methods for determination of stereochemistry and separation of stereoisomers are well-known in the art. In particular embodiments, the stereoisomers of the compounds depicted herein are formed upon treatment with base.

In certain embodiments, the compounds provided herein are “stereochemically pure.” A stereochemically pure compound or has a level of stereochemical purity that would be recognized as “pure” by those of skill in the art. Of course, this level of purity will be less than 100%. In certain embodiments, “stereochemically pure” designates a compound that is substantially free of alternate isomers. In particular embodiments, the compound is 85%, 90%, 91%, 92%, 93%, 94%, 95%, 96%, 97%, 98%, 99%, 99.5% or 99.9% free of other isomers.

As used herein, the term “label” refers to a display of written, printed or graphic matter upon the immediate container of an article, for example the written material displayed on a vial containing a pharmaceutically active agent.

As used herein, the term “labeling” refers to all labels and other written, printed or graphic matter upon any article or any of its containers or wrappers or accompanying such article, for example, a package insert or instructional videotapes or DVDs accompanying or associated with a container of a pharmaceutically active agent.

HCV Treatment

It has surprisingly been found that a narrow group of p38MAP kinase inhibitors are potent HCV entry inhibitors. Without being limited to a particular mechanism of action, compounds provided herein are capable of inhibiting the entry of HCV into a host cell by interacting with SR-B1, the host hepatocyte cell membrane protein involved in the docking and entry of HCV into the host. Accordingly, provided herein are HCV entry inhibitors and pharmaceutical compositions thereof, for use in treatment or prevention of HCV infection in a subject in need thereof. Methods of treatment are described in detail in the sections below. The compound may be any compound provided herein as described in the sections below. In certain embodiments, the compound is in the form of a pharmaceutical composition or dosage form, as described in the sections below.

Current therapy for HCV, as mentioned above, is co-administration of interferon and ribavirin. It is believed that the current therapy operates by modulation of the immune system of a subject to treat or prevent infection by HCV. Operating by a novel mechanism, the compounds, compositions and methods herein offer a novel therapy for the treatment or prevention of HCV infection. As such they can be advantageous for any subject infected with, or at risk for infection with, HCV and particularly for subjects that have not responded to current therapy.

In certain embodiments, the subject may be any subject infected with, or at risk for infection with, HCV. Infection or risk for infection can be determined according to any technique deemed suitable by the practitioner of skill in the art. Particularly preferred subjects are humans infected with HCV.

The HCV can be any HCV known to those of skill in the art. There are at least six genotypes and at least 50 subtypes of HCV currently known to those of skill in the art. The HCV can be of any genotype or subtype known to those of skill. In certain embodiments, the HCV is of a genotype or subtype not yet characterized. In certain embodiments, the subject is infected with HCV of a single genotype. In certain embodiments, the subject is infected with HCV of multiple subtypes or multiple genotypes.

In certain embodiments, the HCV is genotype 1 and can be of any subtype. For instance, in certain embodiments, the HCV is subtype 1a, 1b or 1c. It is believed that HCV infection of genotype 1 responds poorly to current interferon therapy. In certain embodiments, the methods provided herein are advantageous for therapy of HCV infection with genotype 1.

In certain embodiments, the HCV is other than genotype 1. In certain embodiments, the HCV is genotype 2 and can be of any subtype. For instance, in certain embodiments, the HCV is subtype 2a, 2b or 2c. In certain embodiments, the HCV is genotype 3 and can be of any subtype. For instance, in certain embodiments, the HCV is subtype 3a, 3b or 10a. In certain embodiments, the HCV is genotype 4 and can be of any subtype. For instance, in certain embodiments, the HCV is subtype 4a. In certain embodiments, the HCV is genotype 5 and can be of any subtype. For instance, in certain embodiments, the HCV is subtype 5a. In certain embodiments, the HCV is genotype 6 and can be of any subtype. For instance, in certain embodiments, the HCV is subtype 6a, 6b, 7b, 8b, 9a or 11a. See, e.g., Simmonds, 2004, J Gen Virol. 85:3173-88; Simmonds, 2001, J. Gen. Virol., 82, 693-712, the contents of which are incorporated by reference in their entirety.

In certain embodiments, the subject has never received therapy or prophylaxis for HCV infection. In further embodiments, the subject has previously received therapy or prophylaxis for HCV infection. For instance, in certain embodiments, the subject has not responded to HCV therapy. Indeed, under current interferon therapy, up to 50% or more HCV subjects do not respond to therapy. In certain embodiments, the subject can be a subject that received therapy but continued to suffer from viral infection or one or more symptoms thereof. In certain embodiments, the subject can be a subject that received therapy but failed to achieve a sustained virologic response. In certain embodiments, the subject has received therapy for HCV infection but has failed show a 2 log₁₀ decline in HCV RNA levels after 12 weeks of therapy. It is believed that subjects who have not shown more than 2 log₁₀ reduction in serum HCV RNA after 12 weeks of therapy have a 97-100% chance of not responding. Because the compounds provided herein act by mechanism other than current HCV therapy, it is believed that compounds provided herein should be effective in treating such non-responders.

In certain embodiments, the subject is a subject that discontinued HCV therapy because of one or more adverse events associated with the therapy. In certain embodiments, the subject is a subject where current therapy is not indicated. For instance, certain therapies for HCV are associated with neuropsychiatric events. Interferon (IFN)-alfa plus ribavirin is associated with a high rate of depression. Depressive symptoms have been linked to a worse outcome in a number of medical disorders. Life-threatening or fatal neuropsychiatric events, including suicide, suicidal and homicidal ideation, depression, relapse of drug addiction/overdose, and aggressive behavior have occurred in subjects with and without a previous psychiatric disorder during HCV therapy. Interferon-induced depression is a limitation for the treatment of chronic hepatitis C, especially for subjects with psychiatric disorders. Psychiatric side effects are common with interferon therapy and responsible for about 10% to 20% of discontinuations of current therapy for HCV infection.

Accordingly, provided herein are methods of treating or preventing HCV infection in subjects where the risk of neuropsychiatric events, such as depression, contraindicates treatment with current HCV therapy. In some embodiments, provided herein are methods of treating or preventing HCV infection in subjects where a neuropsychiatric event, such as depression, or risk of such indicates discontinuation of treatment with current HCV therapy. In some embodiments, provided herein are methods of treating or preventing HCV infection in subjects where a neuropsychiatric event, such as depression, or risk of such indicates dose reduction of current HCV therapy.

Current therapy is also contraindicated in subjects that are hypersensitive to interferon or ribavirin, or both, or any other component of a pharmaceutical product for administration of interferon or ribavirin. Current therapy is not indicated in subjects with hemoglobinopathies (e.g., thalassemia major, sickle-cell anemia) and other subjects at risk from the hematologic side effects of current therapy. Common hematologic side effects are include bone marrow suppression, neutropenia and thrombocytopenia. Furthermore, ribavirin is toxic to red blood cells and is associated with hemolysis. Accordingly, provided herein are methods of treating or preventing HCV infection in subjects hypersensitive to interferon or ribavirin, or both, subjects with a hemoglobinopathy, for instance thalassemia major subjects and sickle-cell anemia subjects, and other subjects at risk from the hematologic side effects of current therapy.

In certain embodiments, the subject has received HCV therapy and discontinued that therapy prior to administration of a method provided herein. In further embodiments, the subject has received therapy and continues to receive that therapy along with administration of a method provided herein. The methods herein may be co-administered with other therapy for HCV according to the judgment of one of skill in the art. In some embodiments, the methods or compositions provided herein may be co-administered with a reduced dose of the other therapy for HCV.

In certain embodiments, a compound of formula I as provided herein is co-administered with an antiviral agent selected from the group consisting of a nucleoside polymerase inhibitor, a non-nucleoside polymerase inhibitor, a protease inhibitor, a cyclophilin modulator, an interferon and ribavirin.

In one embodiment, a compound of formula I is administered with an interferon.

In one embodiment, a compound of formula I is administered with ribavirin.

In another embodiment, a compound of formula I is administered with an interferon and ribavirin.

In certain embodiments, provided herein are methods of treating a subject that is refractory to treatment with interferon. For instance, in some embodiments, the subject can be a subject that has failed to respond to treatment with one or more agents selected from the group consisting of interferon, interferon α, pegylated interferon α, interferon plus ribavirin, interferon α plus ribavirin and pegylated interferon α plus ribavirin. In some embodiments, the subject can be a subject that has responded poorly to treatment with one or more agents selected from the group consisting of interferon, interferon α, pegylated interferon α, interferon plus ribavirin, interferon α plus ribavirin and pegylated interferon α plus ribavirin.

In further embodiments, provided herein are methods of treating HCV infection in subjects that are pregnant or might get pregnant since current therapy is also contraindicated in pregnant women.

In certain embodiments, the subject has, or is at risk for, co-infection of HCV with HIV. For instance, in the United States, 30% of HIV subjects are co-infected with HCV and evidence indicates that people infected with HIV have a much more rapid course of their hepatitis C infection. Maier and Wu, 2002, World J Gastroenterol 8:577-57. The methods provided herein may be used to treat or prevent HCV infection in such subjects. It is believed that elimination of HCV in these subjects will lower mortality due to end-stage liver disease. Indeed, the risk of progressive liver disease is higher in subjects with severe AIDS-defining immunodeficiency than in those without. See, e.g., Lesens et al., 1999, J Infect Dis 179:1254-1258.

In certain embodiments, the methods or compositions provided herein are administered to a subject following liver transplant. Hepatitis C is a leading cause of liver transplantation in the U.S., and many subjects that undergo liver transplantation remain HCV positive following transplantation. Accordingly, provided herein are methods of treating such recurrent HCV subjects with a compound or composition provided herein. In certain embodiments, a subject is treated according to the methods provided herein before, during or following liver transplant to prevent recurrent HCV infection.

Compounds

Provided herein are methods of treating or preventing hepatitis C infection in a subject in need thereof by administering to the subject an effective amount of an oxoacetamide compound of general formula (I) or (II), or a stereoisomer thereof, or a pharmaceutically acceptable salt thereof:

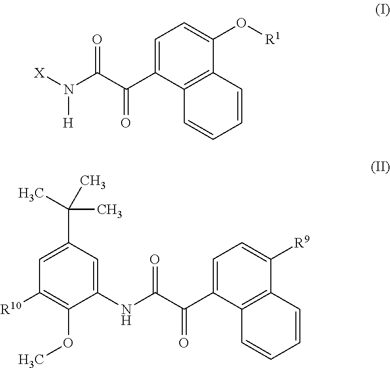

wherein

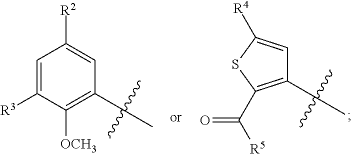

X is

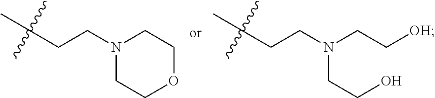

R¹ is methyl, ethyl, isopropyl,
R² is C₁-C₈ alkyl; C₅-C₈ cycloalkyl, or C₇-C₁₀ arylalkyl;
R³ is hydrogen, cyano, —CONHR⁶, —NHSO₂R⁷ or —SO₂N(R⁸)₂;
R⁴ is C₁-C₄ alkyl;
R⁵ is C₁-C₄ alkoxy or —N(R⁸)₂;
R⁶ is 2-pyridyl or C₁-C₆ alkyl, wherein one or more carbon atoms is optionally replaced by an oxygen atom;
R⁷ is C₁-C₄ alkyl, CH₂CF₃, benzyl or phenyl; and
R⁸ is C₁-C₄ alkyl;
R⁹ is bromo or 6-(methylamino)pyridin-3-yl;
R¹⁰ is hydrogen or —CONHR¹¹; and
R¹¹ is hydrogen or C₁-C₄ alkyl;
provided that if R³ is —NHSO₂R⁷ and R⁷ is methyl, then R¹ is not methyl;
provided that if R¹⁰ is hydrogen, R⁹ is 6-(methylamino)pyridin-3-yl; and
provided that if R¹¹ is cyclopropyl, R⁹ is bromo.

In one embodiment, it is further provided that if R² is methyl, then R³ is not —NHSO₂R⁹.

In another embodiment, it is further provided that if R² is methyl and R³ is —NHSO₂R⁹, then R⁹ is not methyl.

In another embodiment, it is further provided that if R³ is —NHSO₂R⁹, then R² is not methyl.

In another embodiment, it is further provided that if R³ is —NHSO₂R⁹ and R⁹ is methyl, R² is not methyl.

In certain embodiments, R¹ is methyl, ethyl, isopropyl or

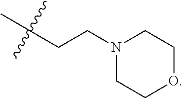

In one embodiment, R¹ is methyl,

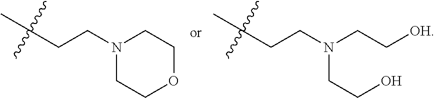

In one embodiment, R¹ is isopropyl or

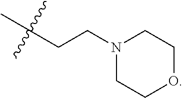

In one embodiment, R¹ is

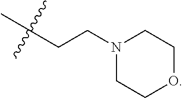

In some embodiments, R² is tert-butyl, cyclohexyl or 1-methyl-1-phenylethyl.

In some embodiments, R² is tert-butyl

In some embodiments, R³ is hydrogen.

In some embodiments, R³ is —NHSO₂R⁹ and R⁹ is methyl.

In some embodiments, if R² is methyl, R³ is not —NHSO₂R⁹.

In one embodiment, if R² is methyl and R³ is —NHSO₂R⁹, R⁹ is not methyl.

In one embodiment, if R³ is —NHSO₂R⁹, R² is not methyl.

In another embodiment, if R³ is —NHSO₂R⁹ and R⁹ is methyl, R² is not methyl

In some embodiments, R⁴ is tert-butyl.

In one embodiment, R⁵ is methoxy or dimethylamino.

In some embodiments, R⁶ is methyl, ethyl, propyl, methoxyethyl, methylenecyclopropyl or 2-pyridyl.

In one embodiment, R⁷ is methyl or ethyl.

In one embodiment, each R⁸ is methyl.

In one embodiment, R¹¹ is methyl or ethyl.

In one embodiment, R⁹ is 6-(methylamino)pyridin-3-yl.

In one embodiment R⁹ is bromo; R¹⁰ is —CONHR¹¹; and R¹¹ is C₁-C₄ alkyl.

In one embodiment R⁹ is bromo; R¹⁰ is —CONHR¹¹; and R¹¹ is C₁-C₄ alkyl.

In one embodiment, the compound, or a pharmaceutically acceptable salt thereof, has the formula:

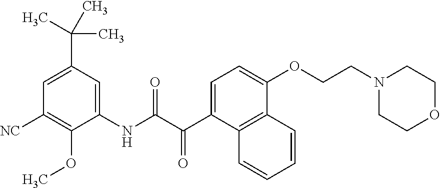

In one embodiment, the compound, or a pharmaceutically acceptable salt thereof, has the formula:

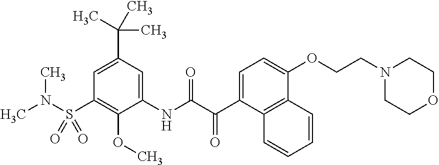

In one embodiment, the compound, or a pharmaceutically acceptable salt thereof, has the formula:

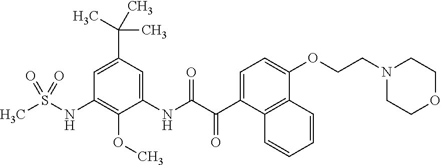

In one embodiment, the compound, or a pharmaceutically acceptable salt thereof, has the formula:

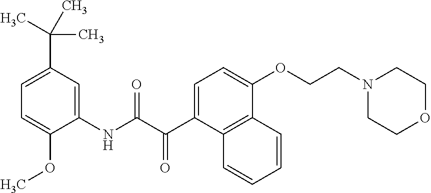

In one embodiment, the compound, or a pharmaceutically acceptable salt thereof, has the formula:

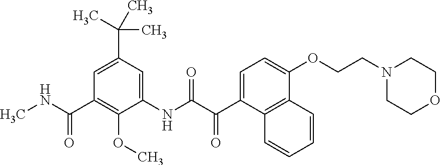

In one embodiment, the compound, or a pharmaceutically acceptable salt thereof, has the formula:

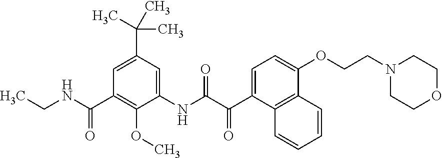

In one embodiment, the compound, or a pharmaceutically acceptable salt thereof, has the formula:

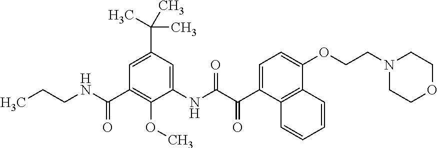

In one embodiment, the compound, or a pharmaceutically acceptable salt thereof, has the formula:

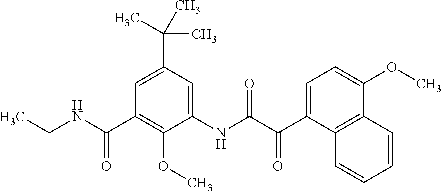

In one embodiment, the compound, or a pharmaceutically acceptable salt thereof, has the formula:

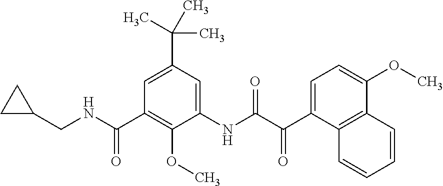

In one embodiment, the compound, or a pharmaceutically acceptable salt thereof, has the formula:

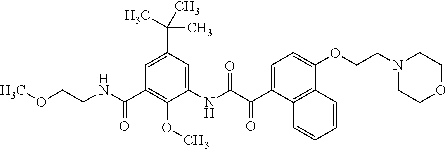

In one embodiment, the compound, or a pharmaceutically acceptable salt thereof, has the formula:

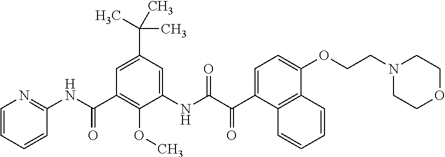

In one embodiment, the compound, or a pharmaceutically acceptable salt thereof, has the formula:

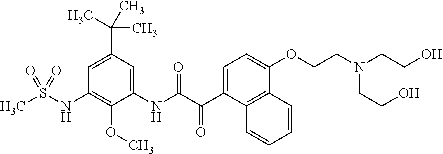

In one embodiment, the compound, or a pharmaceutically acceptable salt thereof, has the formula:

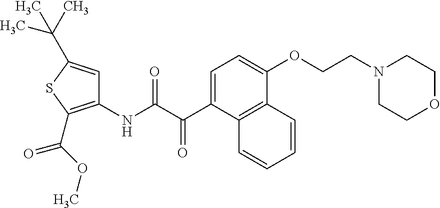

In one embodiment, the compound, or a pharmaceutically acceptable salt thereof, has the formula:

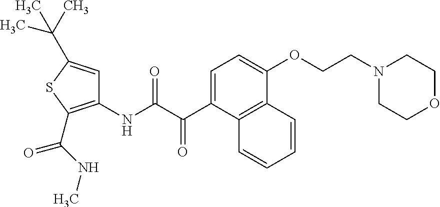

In one embodiment, the compound, or a pharmaceutically acceptable salt thereof, has the formula:

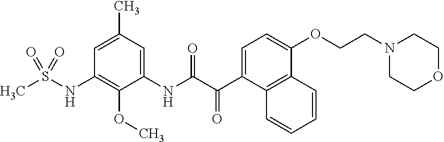

In one embodiment, the compound, or a pharmaceutically acceptable salt thereof, has the formula:

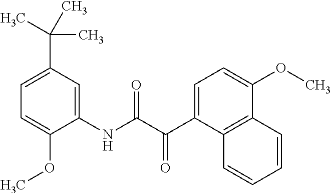

In one embodiment, the compound, or a pharmaceutically acceptable salt thereof, has the formula:

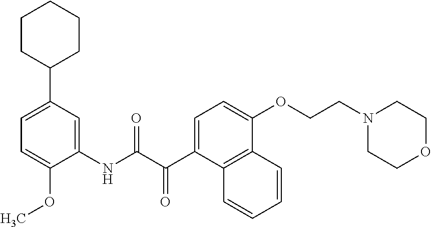

In one embodiment, the compound, or a pharmaceutically acceptable salt thereof, has the formula:

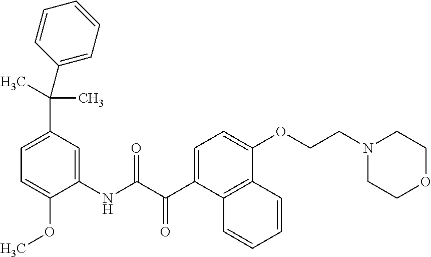

In one embodiment, the compound, or a pharmaceutically acceptable salt thereof, has the formula:

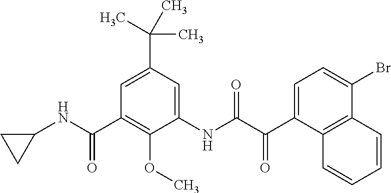

In one embodiment, the compound, or a pharmaceutically acceptable salt thereof, has the formula:

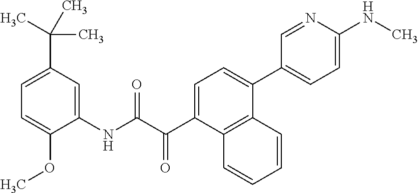

In one embodiment, the compound, or a pharmaceutically acceptable salt thereof, has the formula:

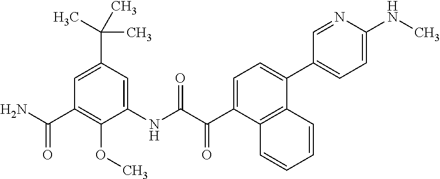

In one embodiment, the compound, or a pharmaceutically acceptable salt thereof, has the formula:

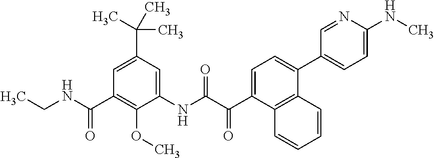

In one embodiment, the compound, or a pharmaceutically acceptable salt thereof, has the formula:

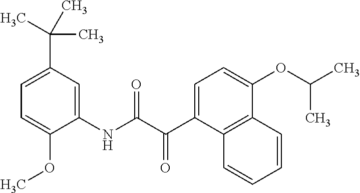

In one embodiment, the compound, or a pharmaceutically acceptable salt thereof, has the formula:

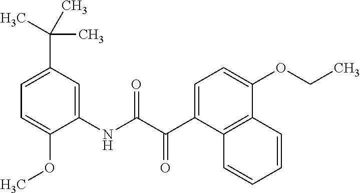

Certain compounds provided herein have previously been disclosed in U.S. Patent Publication No. 2005/0107399, the disclosure of which is incorporated herein in its entirety. U.S. Patent Publication No. 2005/0107399 does not disclose the treatment of HCV with entry inhibitors. U.S. Patent Publication No. 2005/0107399 does not identify or select the particular compounds provided herein for the treatment of HCV.

Preparation of Compounds

The compounds provided herein may be prepared, isolated or obtained by any method apparent to those of skill in the art. Exemplary methods of preparation of the compounds of formula I are provided below.

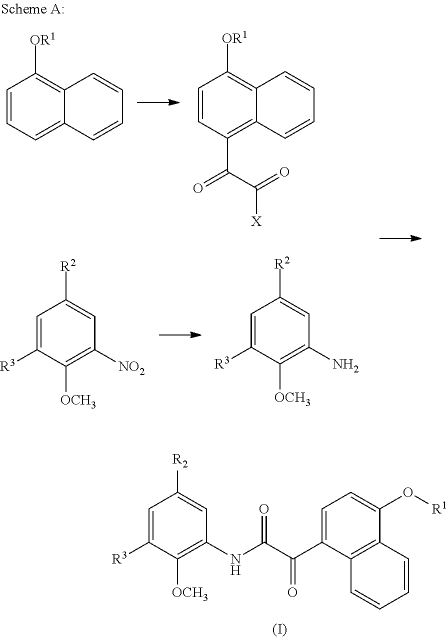

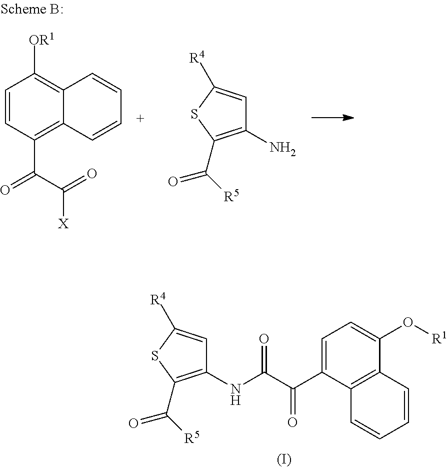

Compounds of formula I may be prepared according to general schemes A and B above.

Specific intermediates in the preparation of the compounds of formula I may be prepared as provided below. The following schemes are merely exemplary methods for the preparation of the compounds of formula I not meant to limit the present invention. Those of skill in the art understand that alternative methods for the preparation of the compounds of formula I exist according to processes known in the art.

### Synthesis of naphthalen-1-yl-oxo-acetic acid derivatives

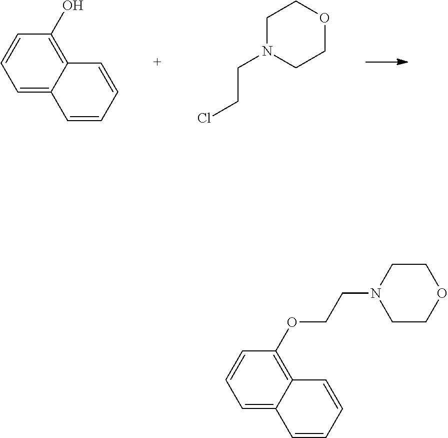

4-[2-(Napthalene-1-yloxy)-ethyl]-morpholine may be prepared, for example, by the reaction of 2-hydroxynapthalene with 2-chloroethyl-morpholine in the presence of base.

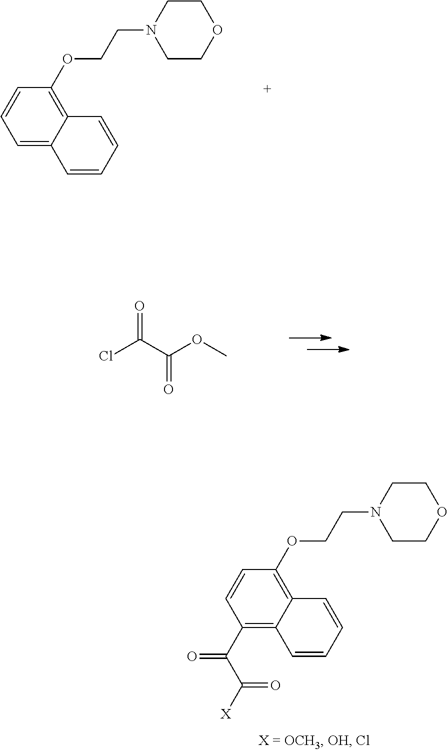

Treatment of 4-[2-(Napthalene-1-yloxy)-ethyl]morpholine with methylchloroglyoxylate in the presence of AlCl₃ provides 4-[2-(morpholin-4-yl-ethoxy-napthalene-1-yl)-oxo-acetic acid methyl or ethyl ester, which may be reacted with hydroxide base to provide the corresponding carboxylic acid, or HCl to provide the corresponding carboxylic acid, and subsequently with oxalyl chloride to produce the corresponding acid chloride. These resulting naphthalen-1-yl-oxo-acetic acid derivatives may be further reacted with substituted anilines or 3-amino-thiophenes to provide the compounds of formula I.

Exemplary Substituted Aniline Intermediates:

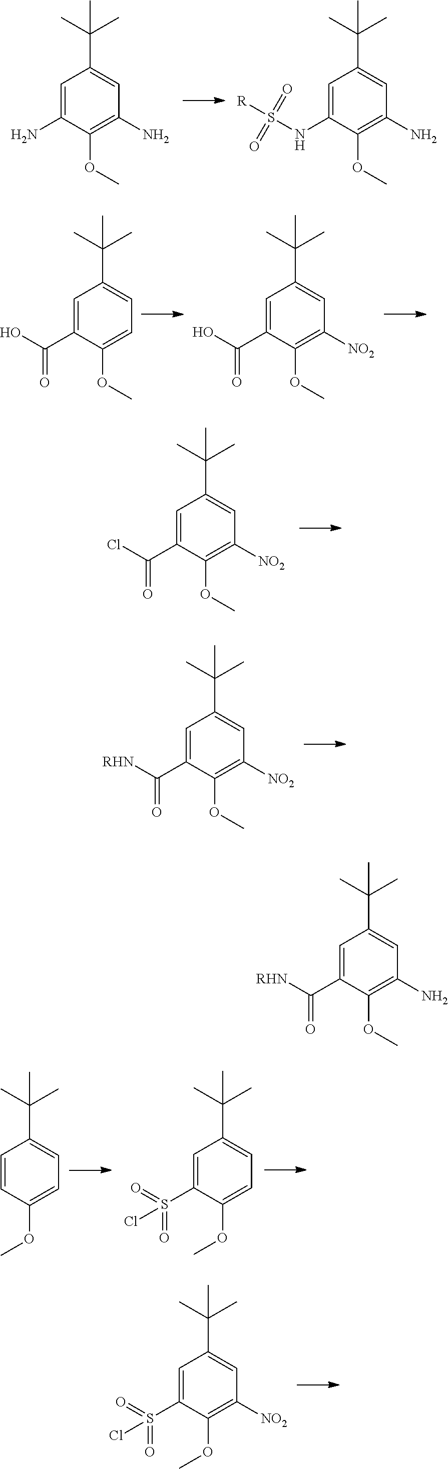

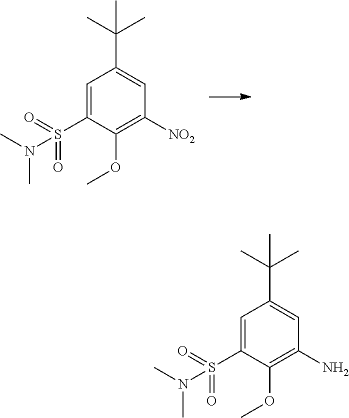

Preparation of the above substituted aniline compounds may be done by methods known in the art using commercially available reagents. For example, 5-tert-butyl-2-methoxy-1,3-benzenediamine is available from Sigma-Aldrich Corp. (St. Louis, Mo.), 5-tert-butyl-2-methoxybenzoic acid is available from Chemos GmbH (Regenstauf, Germany) and 4-tert-butylanisole is available from Acros Organics (ThermoFisher Scientific Inc., Waltham, Mass.). The substituted aniline intermediates may then be employed as provided in scheme A to yield compounds of formula I above.

Exemplary 3-Amino-Thiophene Intermediates:

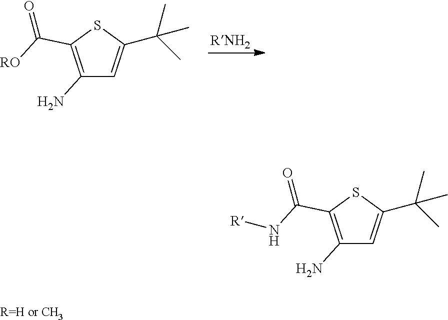

Substituted 3-amino-thiophene compounds as provided above are commercially available or may be prepared by methods known in the art using commercially available reagents. For example, methyl 5-tert-butylthiophene-2-carboxylate and 3-tert-butoxycarbonylamino-5-tert-butylthiophene-2-carboxylic acid are commercially available from Fluorochem Ltd. (Derbyshire, UK) and ChemPur GmbH (Karlsruhe, Germany). These intermediates may then be employed as provided in scheme B to yield compounds of formula I above.

### Preparation of N-substituted-5-tert-butyl-2-methoxy-3-(2-(4-methoxynaphthalen-1-yl)-2-oxoacetamido)benzamides

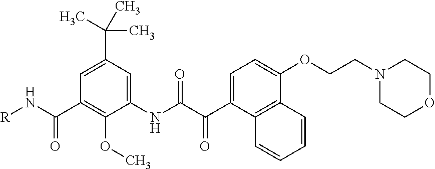

N-substituted-5-tert-butyl-2-methoxy-3-(2-(4-methoxynaphthalen-1-yl)-2-oxoacetamido)benzamides may be prepared as described below by the general procedure of Scheme A.

5-tert-Butyl-2-methoxy-3-nitrobenzoic acid is prepared from 5-t-butyl-2-methoxybenzoic acid using, for example, nitric acid in the presence of acetic acid.

5-tert-Butyl-2-methoxy-3-nitrobenzoic acid is then treated with oxalyl chloride followed by an amine to yield a N-substituted-5-tert-Butyl-2-methoxy-3-nitro-benzamide, which may be reduced with Pd/C and H₂ to provide the corresponding N-substituted-5-tert-Butyl-2-methoxy-3-amino-benzamide.

The N-substituted-5-tert-butyl-2-methoxy-3-(2-(4-methoxynaphthalen-1-yl)-2-oxoacetamido)benzamide is obtained by reaction of the N-substituted-5-tert-Butyl-2-methoxy-3-amino-benzamide from the previous step with 4-[2-(morpholin-4-yl-ethoxy-napthalene-1-yl)-oxo-acetic acid chloride (prepared as shown above).

### Preparation of Compounds of Formula (II)

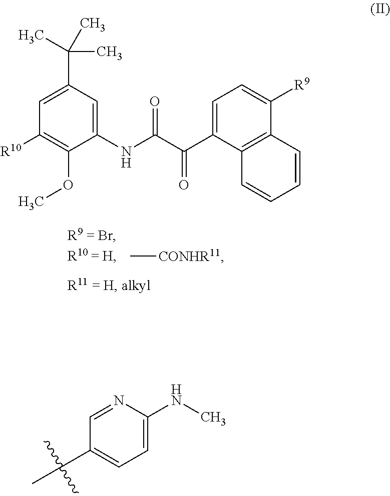

N-substituted-5-tert-butyl-2-methoxy-3-(2-(4-(6-(methylamino)pyridin-3-yl)-naphthalen-1-yl)-2-oxoacetamido)benzamides of formula (II) may be prepared as described below by the general procedure of Scheme C.

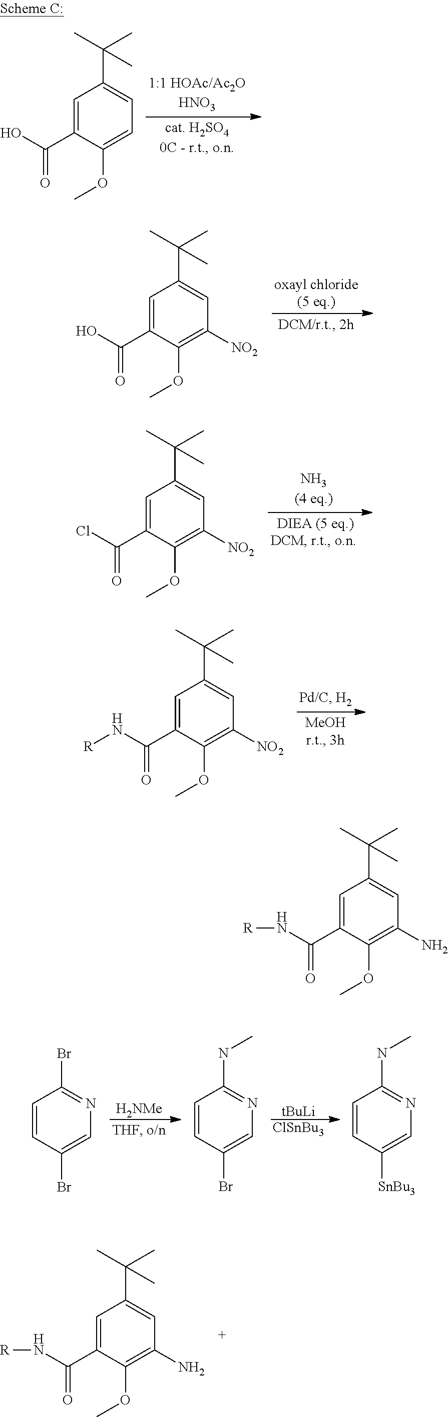

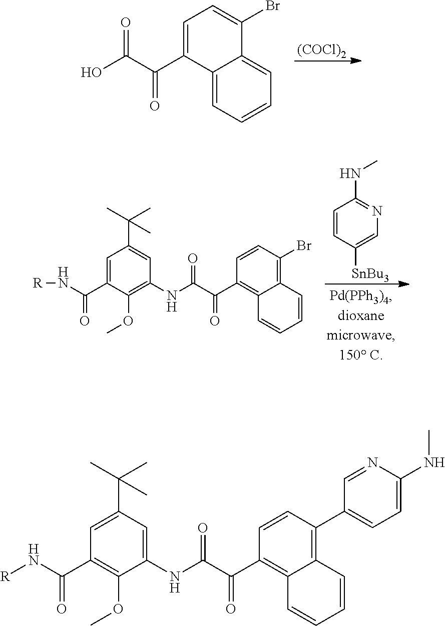

As shown above, N-substituted-5-tert-butyl-2-methoxy-3-(2-bromo-naphthalen-1-yl)-2-oxoacetamido)benzamides of formula (II) may be prepared according to Scheme C, as the bromide compounds are intermediates in the preparation of the corresponding N-substituted-5-tert-butyl-2-methoxy-3-(2-(4-(6-(methylamino)pyridin-3-yl)-naphthalen-1-yl)-2-oxoacetamido)benzamide products above.

Pharmaceutical Salts

A compound provided herein may be in a neutral form or a salt form. The salt form may be any salt form known to those of skill in the art.

Where a compound provided herein is substituted with a basic moiety, an acid addition salt can be formed. The acid which can be used to prepare an acid addition salt includes preferably that which produces, when combined with the free base, a pharmaceutically acceptable salt, that is, a salt whose anion is non-toxic to a subject in the pharmaceutical doses of the salt.

Pharmaceutically acceptable salts include, but are not limited to, those derived from the following acids: mineral acids such as hyrochloric acid, hydrobromic acid, sulfuric acid, phosphoric acid, sulfamic acid and nitric acid; and organic acids such as acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids.

The corresponding acid addition salts include hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

Also provided herein are acid addition salts of the compounds of formula (I), which may be prepared by reaction of the free base with the appropriate acid, by the application or adaptation of known methods. For example, the acid addition salts may be prepared either by dissolving the free base in aqueous or aqueous-alcohol solution or other suitable solvents containing the appropriate acid and isolating the salt by evaporating the solution, or by reacting the free base and acid in an organic solvent, in which case the salt separates directly or can be obtained by concentration of the solution.

The acid addition salts of the compounds provided herein may be regenerated from the salts by the application or adaptation of known methods. For example, parent compounds may be regenerated from their acid addition salts by treatment with an alkali, e.g., aqueous sodium bicarbonate solution or aqueous ammonia solution.

Where a compound provided herein is substituted with an acid moiety, base addition salts can be formed. Pharmaceutically acceptable salts, including for example alkali and alkaline earth metal salts, are those derived from the following bases: sodium hydride, sodium hydroxide, potassium hydroxide, calcium hydroxide, magnesium hydroxide, aluminum hydroxide, lithium hydroxide, zinc hydroxide, barium hydroxide, and organic amines such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Metal salts of the compounds provided herein may be obtained by contacting a hydride, hydroxide, carbonate or similar reactive compound of the chosen metal in an aqueous or organic solvent with the free acid form of the compound. The aqueous solvent employed may be water or it may be a mixture of water with an organic solvent, preferably an alcohol such as methanol or ethanol, a ketone such as acetone, an aliphatic ether such as tetrahydrofuran, or an ester such as ethyl acetate. Such reactions are normally conducted at ambient temperature but they may, if desired, be conducted with heating.

Amine salts of compounds provided herein may be obtained by contacting an amine in an aqueous or organic solvent with the free acid form of the compound. Suitable aqueous solvents include water and mixtures of water with alcohols such as methanol or ethanol, ethers such as tetrahydrofuran, nitriles, such as acetonitrile, or ketones such as acetone. Amino acid salts may be similarly prepared.

The base addition salts of the compounds provided herein may be regenerated from the salts by the application or adaptation of known methods. For example, parent compounds may be regenerated from their base addition salts by treatment with an acid, e.g., hydrochloric acid.

### Pharmaceutical Compositions and Methods of Administration

The compounds used in the methods provided herein may be provided using pharmaceutical compositions containing at least one compound of general formula (I), or a pharmaceutically acceptable salt thereof, either used alone or in the form of a combination with one or more compatible and pharmaceutically acceptable carriers, such as diluents or adjuvants, or with another anti-HCV agent. In clinical practice the compounds provided herein may be administered by any conventional route, in particular orally, parenterally, rectally or by inhalation (e.g. in the form of aerosols). The compounds provided herein are preferably administered orally.

Use may be made, as solid compositions for oral administration, of tablets, pills, hard gelatin capsules, powders or granules. In these compositions, the active product is mixed with one or more inert diluents or adjuvants, such as sucrose, lactose or starch.

These compositions can comprise substances other than diluents, for example a lubricant, such as magnesium stearate, or a coating intended for controlled release.

Use may be made, as liquid compositions for oral administration, of solutions which are pharmaceutically acceptable, suspensions, emulsions, syrups and elixirs containing inert diluents, such as water or liquid paraffin. These compositions can also comprise substances other than diluents, for example wetting, sweetening or flavoring products.

The compositions for parenteral administration can be emulsions or sterile solutions. Use may be made, as solvent or vehicle, of propylene glycol, a polyethylene glycol, vegetable oils, in particular olive oil, or injectable organic esters, for example ethyl oleate. These compositions can also contain adjuvants, in particular wetting, isotonizing, emulsifying, dispersing and stabilizing agents. Sterilization can be carried out in several ways, for example using a bacteriological filter, by radiation or by heating. They can also be prepared in the form of sterile solid compositions which can be dissolved at the time of use in sterile water or any other injectable sterile medium.

The compositions for rectal administration are suppositories or rectal capsules which contain, in addition to the active principle, excipients such as cocoa butter, semi-synthetic glycerides or polyethylene glycols.

The compositions can also be aerosols. For use in the form of liquid aerosols, the compositions can be stable sterile solutions or solid compositions dissolved at the time of use in apyrogenic sterile water, in saline or any other pharmaceutically acceptable vehicle. For use in the form of dry aerosols intended to be directly inhaled, the active principle is finely divided and combined with a water-soluble solid diluent or vehicle, for example dextran, mannitol or lactose.

In a preferred embodiment, a composition provided herein is a pharmaceutical composition or a single unit dosage form. Pharmaceutical compositions and single unit dosage forms may comprise a prophylactically or therapeutically effective amount of one or more prophylactic or therapeutic agents (e.g., a compound of formula (I), or other prophylactic or therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Lactose free compositions may comprise excipients that are well known in the art and are listed, for example, in the U.S. Pharmacopia (USP)SP (XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

Also provided herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, NY, N.Y., 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms may be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the subject. The formulation should suit the mode of administration. In a preferred embodiment, the pharmaceutical compositions or single unit dosage forms are sterile and in suitable form for administration to a subject, preferably an animal subject, more preferably a mammalian subject, and most preferably a human subject.

The pharmaceutical compositions provided herein are formulated to be compatible with their intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lidocaine to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

The composition, shape, and type of dosage forms will typically vary depending on their use. For example, a dosage form used in the initial treatment of viral infection may contain larger amounts of one or more of the active ingredients it comprises than a dosage form used in the maintenance treatment of the same infection. Similarly, a parenteral dosage form may contain smaller amounts of one or more of the active ingredients it comprises than an oral dosage form used to treat the same disease or disorder. These and other ways in which specific dosage forms will vary from one another will be readily apparent to those skilled in the art. See, e.g., Remington's Pharmaceutical Sciences, 18th ed., Mack Publishing, Easton Pa. (1990).

Generally, the ingredients of the compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms comprise a compound provided herein, or a pharmaceutically acceptable salt, solvate or hydrate thereof, lie within the range of from about 0.1 mg to about 1000 mg per day, given as a single once-a-day dose in the morning but preferably as divided doses throughout the day taken with food. Particular dosage forms of the invention have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active agent.

Oral Dosage Forms

Pharmaceutical compositions provided herein that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

In certain embodiments, the oral dosage forms are solid and prepared under anhydrous conditions with anhydrous ingredients, as described in detail in the sections above. However, other embodiments extend beyond anhydrous, solid oral dosage forms. As such, further forms are described herein.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately mixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

Examples of excipients that can be used in oral dosage forms include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. In some embodiments, the binder or filler in a pharmaceutical composition provided herein is present in from about 50 to about 99 weight percent of the pharmaceutical composition or dosage form.

Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC 581. Suitable anhydrous or low moisture excipients or additives include AVICEL PH 103™ and Starch 1500 LM.

In some embodiments, disintegrants are used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that may be used in pharmaceutical compositions include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that may be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB O SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Delayed Release Dosage Forms

Active ingredients may be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients provided herein. Therefore, provided herein are single unit dosage forms suitable for oral administration such as, but not limited to, tablets, capsules, gelcaps, and caplets that are adapted for controlled release.

All controlled release pharmaceutical products have a common goal of improving drug therapy over that achieved by their non controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased subject compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

Parenteral Dosage Forms

Provided herein are parenteral dosage forms. Parenteral dosage forms can be administered to subjects by various routes including, but not limited to, subcutaneous, intravenous (including bolus injection), intramuscular, and intra-arterial. Because their administration typically bypasses subjects' natural defences against contaminants, parenteral dosage forms are preferably sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into parenteral dosage forms.

Transdermal, Topical & Mucosal Dosage Forms

Also provided herein are transdermal, topical, and mucosal dosage forms. Transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, which are non toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients, including but not limited to the compounds provided herein. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates or solvates of the active ingredients can be used to further adjust the properties of the resulting composition.

Dosage and Unit Dosage Forms

In human therapeutics, the doctor will determine the posology which he considers most appropriate according to a preventive or curative treatment and according to the age, weight, stage of the infection and other factors specific to the subject to be treated. Generally, doses are from about 1 to about 1000 mg per day for an adult, or from about 5 to about 250 mg per day or from about 10 to 50 mg per day for an adult. In certain embodiments, doses are from about 5 to about 400 mg per day, and more preferably 25 to 200 mg per day per adult. Dose rates of from about 50 to about 500 mg per day are also preferred.

In further aspects, provided herein are methods of treating or preventing hepatitis C virus infection in a subject by administering, to a subject in need thereof, an effective amount of a compound of the invention, or a pharmaceutically acceptable salt thereof, with a high therapeutic index against hepatitis C virus. The therapeutic index can be measured according to any method known to those of skill in the art, such as the method described in the examples below. In certain embodiments, the therapeutic index is the ratio of a concentration at which the compound is toxic, to the concentration that is effective against hepatitis C virus. Toxicity can be measured by any technique known to those of skill including cytotoxicity (e.g. IC₅₀ or IC₉₀) and lethal dose (e.g. LD₅₀ or LD₉₀). Likewise, effective concentrations can be measured by any technique known to those of skill including effective concentration (e.g. EC₅₀ or EC₉₀) and effective dose (e.g. ED₅₀ or ED₉₀). Preferably, similar measurements are compared in the ratio (e.g. IC₅₀/EC₅₀, IC₉₀/EC₉₀, LD₅₀/ED₅₀ or LD₉₀/ED₉₀). In certain embodiments, the therapeutic index can be as high as 2.0, 5.0, 10.0, 15.0, 20.0, 25.0, 50.0, 75.0, 100.0, 125.0, 150.0 or higher.

The amount of the compound or composition which will be effective in the prevention, treatment, management, or amelioration of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

Exemplary doses of a composition include milligram or microgram amounts of the active compound per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions provided herein, the dosage administered to a subject is typically 0.140 mg/kg to 3 mg/kg of the subject's body weight, based on weight of the active compound. In one embodiment, the dosage administered to a subject is between 0.20 mg/kg and 2.00 mg/kg, or between 0.30 mg/kg and 1.50 mg/kg of the subject's body weight.

In one embodiment, the recommended daily dose range of a composition provided herein for the treatment or prevention of a HCV infection lie within the range of from about 0.1 mg to about 1000 mg per day, given as a single once-a-day dose or as divided doses throughout a day. In one embodiment, the daily dose is administered twice daily in equally divided doses. Specifically, a daily dose range should be from about 10 mg to about 200 mg per day, more specifically, between about 10 mg and about 150 mg per day, or even more specifically between about 25 and about 100 mg per day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with a composition provided herein are also encompassed by the above described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In a specific embodiment, the dosage of the composition provided herein, based on weight of the active compound, administered to prevent or treat a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition administered to prevent, treat, manage, or ameliorate a disorder, or one or more symptoms thereof in a subject is a unit dose of 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In certain embodiments, treatment or prevention can be initiated with one or more loading doses of a compound or composition provided herein followed by one or more maintenance doses. In such embodiments, the loading dose can be, for instance, about 60 to about 400 mg per day, or about 100 to about 200 mg per day for one day to five weeks. The loading dose can be followed by one or more maintenance doses. Each maintenance does can be, independently, about from about 10 mg to about 200 mg per day, more specifically, between about 25 mg and about 150 mg per day, or even more specifically between about 25 and about 80 mg per day. Maintenance doses are preferably administered daily and can be administered as single doses, or as divided doses.

In certain embodiments, a dose may be administered to achieve a steady-state concentration of the active ingredient in blood or serum of the subject. The steady-state concentration can be determined by measurement according to techniques available to those of skill or can be based on the physical characteristics of the subject such as height, weight and age. In certain embodiments, a sufficient amount of a compound or composition is administered to achieve a steady-state concentration in blood or serum of the subject of from about 300 to about 4000 ng/mL, from about 400 to about 1600 ng/mL, or from about 600 to about 1200 ng/mL. Loading doses can be administered to achieve steady-state blood or serum concentrations of about 1200 to about 8000 ng/mL, or about 2000 to about 4000 ng/mL for one to five days. Maintenance doses can be administered to achieve a steady-state concentration in blood or serum of the subject of from about 300 to about 4000 ng/mL, from about 400 to about 1600 ng/mL, or from about 600 to about 1200 ng/mL.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In certain aspects, provided herein are unit dosages comprising a compound of formula (I), or a pharmaceutically acceptable salt thereof, in a form suitable for administration. Such forms are described in detail above. In certain embodiments, the unit dosage comprises 1 to 1000 mg, 5 to 250 mg or 10 to 50 mg active ingredient. In particular embodiments, the unit dosages comprise about 1, 5, 10, 25, 50, 100, 125, 250, 500 or 1000 mg active ingredient. Such unit dosages can be prepared according to techniques familiar to those of skill in the art.

Kits

Also provided herein are kits for use in methods of treatment or prophylaxis of HCV infection. The kits may include a compound or composition of provided herein with instructions providing information to a health care provider regarding usage for treating or preventing a HCV infection. Instructions may be provided in printed form or in the form of an electronic medium such as a floppy disc, CD, or DVD, or in the form of a website address where such instructions may be obtained. A unit dose of a compound or composition provided herein may include a dosage such that when administered to a subject, a therapeutically or prophylactically effective plasma level of the compound or composition in the subject for at least 1 day. In some embodiments, a compound or composition provided herein may be included as a sterile aqueous pharmaceutical composition or dry powder (e.g., lyophilized) composition. In some embodiments, the compound is according to formula (I).

In some embodiments, suitable packaging is provided. As used herein, “packaging” refers to a solid matrix or material customarily used in a system and capable of holding within fixed limits a compound or composition provided herein suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

Kits provided herein may also comprise, in addition to the compound of formula (I) or a composition thereof, other compounds or compositions for use with compound of formula (I) or composition thereof as described in the methods above.

Combination Therapy

The compounds provided herein may also be combined or used in combination with other therapeutic agents useful in the treatment and/or prevention of an HCV infection.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

As used herein, the term “synergistic” includes a combination of a compound provided herein and another therapy (e.g., a prophylactic or therapeutic agent) which has been or is currently being used to treat, prevent, or manage a disease or disorder, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a disorder. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention or treatment of a disorder). In addition, a synergistic effect can result in improved efficacy of agents in the prevention or treatment of a disorder. Finally, a synergistic effect of a combination of therapies (e.g., a combination of prophylactic or therapeutic agents) may avoid or reduce adverse or unwanted side effects associated with the use of either therapy alone.

The compound provided herein can be administered in combination or alternation with another therapeutic agent, such as an anti-HCV agent. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

It has been recognized that drug-resistant variants of HCV can emerge after prolonged treatment with an antiviral agent. Drug resistance most typically occurs due to the mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameters of the drug can be altered by such combination or alternation therapy. In general, combination therapy is typically preferred over alternation therapy because it induces multiple simultaneous stresses on the virus.

Suitable HCV protease inhibitors include, but not limited to, Medivir HCV protease inhibitor (Medivir/Tobotec); ITMN-191 (InterMune), SCH 503034 (Schering), VX950 (Vertex); substrate-based NS3 protease inhibitors as disclosed in WO 98/22496; Attwood et al., Antiviral Chemistry and Chemotherapy 1999, 10, 259-273; DE 19914474; WO 98/17679; WO 99/07734; non-substrate-based NS3 protease inhibitors, such as 2,4,6-trihydroxy-3-nitro-benzamide derivatives (Sudo et al., Biochem. Biophys. Res. Commun. 1997, 238, 643-647), RD3-4082, RD3-4078, SCH 68631, and a phenanthrenequinone (Chu et al., Tetrahedron Letters 1996, 37, 7229-7232); SCH 351633 (Chu et al., Bioorganic and Medicinal Chemistry Letters 1999, 9, 1949-1952); Eglin c, a potent polymerase inhibitor (Qasim et al., Biochemistry 1997, 36, 1598-1607).

Other suitable protease inhibitors for the treatment of HCV include those disclosed in, for example, U.S. Pat. No. 6,004,933, which discloses a class of cysteine protease inhibitors of HCV endopeptidase 2.

Additional hepatitis C virus NS3 protease inhibitors include those disclosed in, for example, Llinàs-Brunet et al., Bioorg. Med. Chem. Lett. 1998, 8, 1713-1718; Steinkühler et al., Biochemistry 1998, 37, 8899-8905; U.S. Pat. Nos. 5,538,865; 5,990,276; 6,143,715; 6,265,380; 6,323,180; 6,329,379; 6,410,531; 6,420,380; 6,534,523; 6,642,204; 6,653,295; 6,727,366; 6,838,475; 6,846,802; 6,867,185; 6,869,964; 6,872,805; 6,878,722; 6,908,901; 6,911,428; 6,995,174; 7,012,066; 7,041,698; 7,091,184; 7,169,760; 7,176,208; 7,208,600; U.S. Pat. App. Pub. Nos.: 2002/0016294, 2002/0016442; 2002/0037998; 2002/0032175; 2004/0229777; 2005/0090450; 2005/0153877; 2005/176648; 2006/0046956; 2007/0021330; 2007/0021351; 2007/0049536; 2007/0054842; 2007/0060510; 2007/0060565; 2007/0072809; 2007/0078081; 2007/0078122; 2007/0093414; 2007/0093430; 2007/0099825; 2007/0099929; 2007/0105781; WO 98/17679; WO 98/22496; WO 99/07734; WO 00/059929; WO 00/09543; WO 02/060926; WO 02/08187; WO 02/008251; WO 02/008256; WO 02/08198; WO 02/48116; WO 02/48157; WO 02/48172; WO 03/053349; WO 03/064416; WO 03/064456; WO 03/099274; WO 03/099316; WO 2004/032827; WO 2004/043339; WO 2005/037214; WO 2005/037860; WO 2006/000085; WO 2006/119061; WO 2006/122188; WO 2007/001406; WO 2007/014925; WO 2007/014926; and WO 2007/056120.

Other protease inhibitors include thiazolidine derivatives, such as RD-1-6250, RD4 6205, and RD4 6193, which show relevant inhibition in a reverse-phase HPLC assay with an NS3/4A fusion protein and NS5A/5B substrate (Sudo et al., Antiviral Research 1996, 32, 9-18); thiazolidines and benzanilides identified in Kakiuchi et al., FEBS Lett. 1998, 421, 217-220; Takeshita et al., Analytical Biochemistry 1997, 247, 242-246.

Suitable non-nucleoside HCV polymerase inhibitors include, but are not limited to, A-848837 (Abbott), gliotoxin (Ferrari et al., Journal of Virology 1999, 73, 1649-1654), and the natural product cerulenin (Lohmann et al., Virology 1998, 249, 108-118).

Suitable nucleoside HCV polymerase inhibitors include, but are not limited to, R7128 (F. Hoffmann-La Roche Ltd., Basel, Switzerland), PSI-7851 (Pharmasset, Inc., Princeton, N.J.), PSI-352879 (Pharmasset, Inc., Princeton, N.J.), PSI-352938 (Pharmasset, Inc., Princeton, N.J.), IDX184 (Idenix Pharmaceuticals, Inc., Cambridge, Mass.), INX-189 (Inhibitex, Inc., Alpharetta, Ga.), and the compounds described in U.S. Pat. Nos. 6,660,721; 6,777,395; 6,784,166; 6,846,810; 6,914,054; 6,927,291; 7,094,770; 7,105,499; 7,125,855; and 7,202,224; U.S. Pat. Pub. Nos. 2004/0121980; 2005/0009737; 2005/0038240; 2006/0040890; and 2008/0286230; WO 99/43691; WO 01/32153; WO 01/60315; WO 01/79246; WO 01/90121, WO 01/92282, WO 02/18404; WO 02/32920, WO 02/48165, WO 02/057425; WO 02/057287; WO 2004/002422, WO 04/002999, and WO 04/003000.

Suitable cyclophilin modulators include, but are not limited to, a cyclosporin (e.g., cyclosporin A), an anti-cyclophilin antibody (see, e.g., Yang, F. et al., J. Virology, 2008, 82(11):5269-5278), sanglifehrin A, NIM811 (Novartis AG, Basel, Switzerland); DEBIO-025 (Debiopharm SA, Lausanne, Switzerland), and the compounds described in U.S. Pat. No. 7,439,227; U.S. Pat. Pub. Nos. 2007/0275884; 2008/0045454 and 2009/0221598; WO 05/0021028; WO 06/038088; WO 06/071618; WO 06/23394 and WO 08/043,797.

Other miscellaneous compounds that can be used as second agents include, for example, 1-amino-alkylcyclohexanes (U.S. Pat. No. 6,034,134), alkyl lipids (U.S. Pat. No. 5,922,757), vitamin E and other antioxidants (U.S. Pat. No. 5,922,757), squalene, amantadine, bile acids (U.S. Pat. No. 5,846,964), N-(phosphonacetyl)-L-aspartic acid (U.S. Pat. No. 5,830,905), benzenedicarboxamides (U.S. Pat. No. 5,633,388), polyadenylic acid derivatives (U.S. Pat. No. 5,496,546), 2′,3′-dideoxyinosine (U.S. Pat. No. 5,026,687), benzimidazoles (U.S. Pat. No. 5,891,874), plant extracts (U.S. Pat. Nos. 5,725,859; 5,837,257; and 6,056,961), and piperidines (U.S. Pat. No. 5,830,905).

In certain embodiments, one or more compounds provided herein are administered in combination or alternation with an anti-hepatitis C virus interferon, including, but not limited to, natural interferon, INTRON® A (interferon alfa-2b) and PEGASYS® (Peginterferon alfa-2a); ROFERON® A (recombinant interferon alfa-2a), INFERGEN® (interferon alfacon-1), PEG-INTRON® (pegylated interferon alfa-2b), interferon beta-1a, omega interferon, interferon gamma, interferon tau, interferon delta or interferon γ-1b. In one embodiment, the anti-hepatitis C virus interferon is INFERGEN®, IL-29 (PEG-Interferon lambda), R7025 (Maxy-alpha), BELEROFON®, oral interferon alpha, BLX-883 (LOCTERON®), omega interferon, MULTIFERON®, medusa interferon, ALBUFERON®, or REBIF®.

### EXAMPLES

The following examples illustrate the synthesis of representative compounds of formula (I) used in the methods provided herein. The following examples further illustrate the synthesis of intermediates used in the preparation of the compounds of formula (I). These examples are not intended, nor are they to be construed, as limiting the scope of the invention. It will be clear that the invention may be practiced otherwise than as particularly described herein. Numerous modifications and variations of the present invention are possible in view of the teachings herein and, therefore, are within the scope of the invention.

### Example 1

### Preparation of N-(5-tert-butyl-2-methoxyphenyl)-2-{4-[2-(morpholin-4-yl)ethoxy]naphthalen-1-yl}-2-oxoacetamide

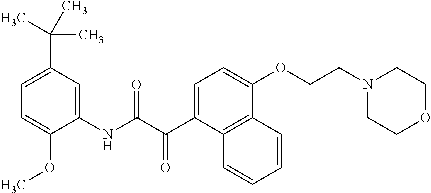

Method A:

### Intermediate 2: 4-[2naphthalen-1-yloxy)ethyl]morpholine

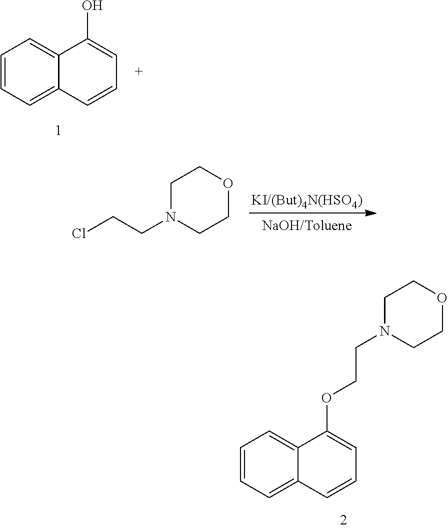

Materials:

| Material | Quantity (kg) | | Molecular Weight | Moles |
|---|---|---|---|---|
| Toluene | 120 | L | 92.14 | |
| 1-Naphthol | 23.7 | kg | 144.17 | 1.02 eq |
| N-Chloroethylmorpholine | 30 | kg | 186.08 | 1 eq |
| hydrochloride | | | | |
| Potassium iodide | 0.6 | kg | 166.0 | 0.02 eq |
| Tetrabutyl ammonium | 2.7 | kg | 339.54 | 0.05 eq |
| hydrogen sulfate | | | | |
| 50% Sodium hydroxide | 64.5 | kg | 40.0 | ~10 eq |
| Ethyl acetate | 45 | L | 88.11 | |
| Heptane | 45 | L | 100.2 | |
| Methylene chloride | 69 | L | 84.93 | |

65 L of demineralized water was added to a vessel charged with 64.5 kg of 50% sodium hydroxide. In a separate vessel charged with 30 kg N-chloroethylmorpholine hydrochloride, 23.7 kg of 1-naphthol, 2.7 kg of tetrabutyl ammonium hydrogen sulfate, 120 L toluene and 0.6 kg potassium iodide were added. The sodium hydroxide solution was added to the vessel charged with the N-chloroethylmorpholine hydrochloride mixture over 20 min, the temperature kept at 20-40° C. The mixture was heated to 90° C. for 3 hours. The mixture was then cooled to 40-45° C. and maintained to reaction completion.

If the reaction completion was less than 95%, the reaction mixture was heated to 90° C. for 1 hour and resampled as before.

102 L of demineralized water was added to the mixture such that the temperature did not rise to more than 50° C. In some cases there were three phases: an orange lower aqueous phase, a black middle phase and a top light green organic phase. The orange aqueous and intermediate phases were separated at 40° C. The organic phase was repeatedly washed with 60 L of demineralized water (15 min, 15-25° C.). Toluene was distilled from the organic phase at ≦20 mbar at 60° C.

The resulting organic phase was cooled to 40° C. under nitrogen atmosphere and 15 L of ethyl acetate and 15 L of heptane were added. The temperature was raised to 60° C. and vacuum was slowly added to distill the solvents. This process was repeated once and sampled to determine toluene content. If the toluene level was greater than 0.3%, the mixture was distilled as before.

The temperature was again raised to 60° C. and vacuum was slowly added to distill the solvents, cooled to 25° C. and 69 L of methylene chloride was added and the mixture stirred 15-20 min. Yield: 35.5 kg, 85.6% (96.0% pure).

### Intermediate 4: {4-[2-(morpholin-4-yl)ethoxy]naphthalen-1-yl}(oxo)acetic acid

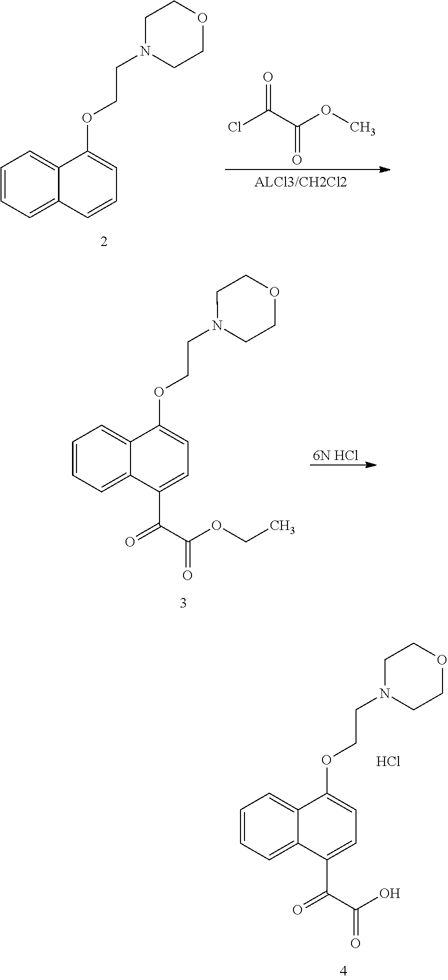

Materials:

| Material | Quantity (kg) | | Molecular Weight |
|---|---|---|---|
| Intermediate 2 in | 31 kg (32.3 × | | 257.3 |
| methylene chloride | 96.03% pure) | | |
| Methyl oxalyl chloride | 39.8 | kg | 122.51 |
| Methylene chloride | 434 | L | 84.9 |
| Aluminum chloride | 43.4 | kg | 134.34 |
| Clarcel DIC (filter aid) | 108.5 | kg | — |
| Hydrochloric acid | 279 | L | 36.5 |
| Methyl-t-butyl ether | 252 | L | 88.2 |
| Acetone | 168 | L | 58.1 |
| Ethanol | 252 | L | |

To 43.4 kg aluminum chloride and 434 L methylene chloride was added 39.8 kg oxalyl chloride with stirring at 10-23° C. The mixture was then rinsed with 62 L methylene chloride and 108.5 kg of Clarcel DIC (filter aid) wa added. 31 kg of intermediate 2 was added over 30 min in methylene chloride. A temperature of 20° C. was maintained for a minimum of 16 hrs. 77.5 kg of demineralized water was slowly added, controlling the temperature at 20-30° C. The resulting mixture was stirred briskly at 20-25° C. for 1-2 hrs.

The resulting solid was filtered and washed with methylene chloride (186 L). Any aqueous phase was separated from the organic phase. The methylene chloride was then distilled at at atmospheric pressure at 55-60° C. until about 500 L remained. 310 L of demineralized water was added and distillation continued at 75-80° C. until the methylene chloride was eliminated. The mixture was cooled to 65-70° C. and 325 kg of hydrochloric acid was added, maintaining the temperature at 76° C. for 16 hrs. Crystallization began after about 30 min. The mixture was then cooled to −2 to 2° C. for at least 3 hrs. The resulting cake was washed by an acid wash solution prepared by mixing 77.5 L of hydrochloric acid and 77.5 L of demineralized water. The cake was then washed three times with 84 L (each) of ethanol, twice with 84 L (each) acetone, and three times with 84 L (each) methyl-t-butyl ether. The cake was dried at not more than 60° C. Yield: 24.6 kg, 55.0% (96% pure).

### N-(5-tert-butyl-2-methoxyphenyl)-2-{4-[2-(morpholin-4-yl)ethoxy]naphthalen-1-yl}-2-oxoacetamide

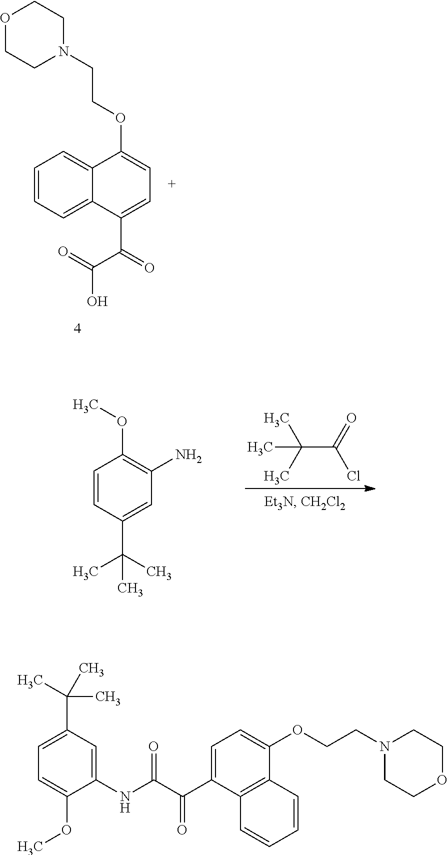

Materials:

| | Quantity | Molecular | | Mole |
|---|---|---|---|---|
| Material | (g) | Weight | Moles | equivalents |
| Intermediate 4 | 2.2 | 365 | 0.006027 | 1.1 |
| Trimethylacetyl | 1.31 | 120 | 0.010917 | 2 |
| chloride | | | | |
| Triethylamine | 1.66 | 101 | 0.016436 | 3 |
| 5-(t-butyl)-2- | 0.98 | 179 | 0.005475 | 1 |
| methoxyaniline | | | | |

2.2 g of intermediate 4 was treated with dichloromethane, 40 ml, and then 1.66 g of triethylamine. The mixture was stirred 1 hour to provide a solution. The solution was then treated by the slow addition of trimethylacetyl chloride, 0.66 g, and then the mixture was stirred 1 hour. The mixture was then rotary vacuum evaporated, and the residue was treated with 30 ml of toluene, and again rotary vacuum evaporated to remove the excess trimethylacetyl chloride. The residue was taken up with dichloromethane, 30 ml, and 5-(t-butyl)-2-methoxyaniline, 0.98 g, dissolved in dichloromethane, 10 ml, was added. The reaction was stirred at room temperature for 1 hour. TLC with 10% methanol/methylene chloride on silica shows conversion to product.

The reaction mix is then washed with an equal volume of 10% K₂CO₃ and the layers separated. The organic layer was again washed with 10% K₂CO₃, and the layers separated. The organic layer is then rotary vacuum evaporated and the residue was treated with methanol, 5 ml, and cooled to 0° C. The resulting solids were collected by filtration, washed with methanol, 5 ml, tert-butyl methyl ether, 5 ml, and air dried to give N-(5-tert-butyl-2-methoxyphenyl)-2-{-4-[2-(morpholin-4-yl)ethoxy]naphthalen-1-yl}-2-oxoacetamide.

### Example 2

### Preparation of N-(5-tert-butyl-2-methoxyphenyl)-2-{4-[2-(morpholin-4-yl)ethoxy]nanhthalen-1-yl}-2-oxoacetamide hydrochloride

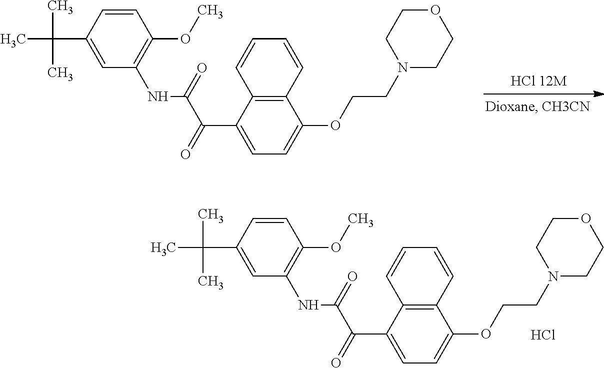

N-(5-tert-butyl-2-methoxyphenyl)-2-{4-[2-(morpholin-4-yl)ethoxy]naphthalen-1-yl}-2-oxoacetamide (1.0 g, 1 eq.) was dissolved in a mixture of 37% HCl (1.0 ml, 5 eq.) and dioxane (10 ml), and the solution stirred for 1-2 hrs. The solution was evaporated until almost dry, and 10 ml of acetonitrile was added to the residue. After 18 hrs at 0° C., a precipitate was provided. Filtration and washing of the solid with acetonitrile (5 ml) and methyl-t-butyl ether (5 ml×2) yielded the final product, N-(5-tert-butyl-2-methoxyphenyl)-2-{4-[2-(morpholin-4-yl)ethoxy]naphthalen-1-yl}-2-oxoacetamide hydrochloride.

### Method B:

Prepare intermediate 2 as shown in Method A.

### Intermediate 4: {4-[2-(morpholin-4-yl)ethoxy]naphthalen-1-yl}(oxo)acetic acid

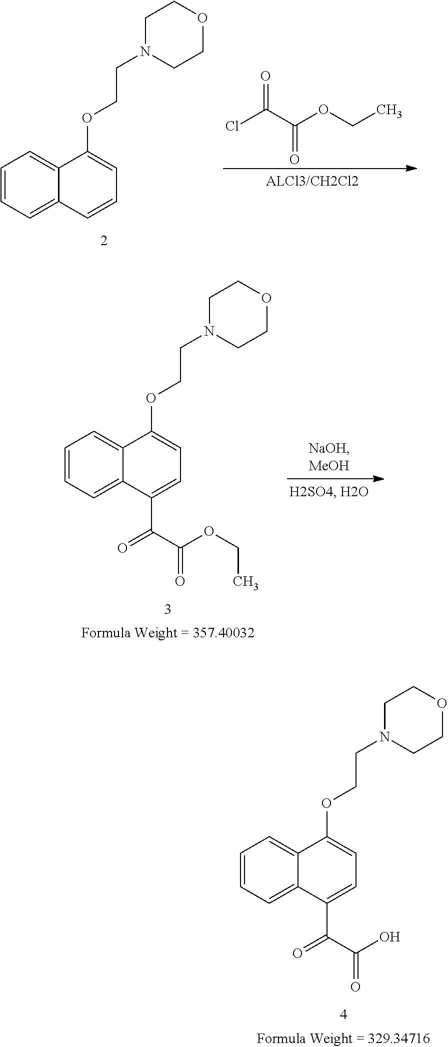

In a 1 L flask was placed a 7 cm 30 gram magnetic stirring bar, 32 g of celite 545 was added followed by dichloromethane, filling to the 700 ml mark. Stirring was started, 14.5 g of aluminum chloride was added, followed by 11.8 ml of ethyl chlorooxoacetate. The mixture was stirred 10 minutes, and then a solution of 10.55 g of intermediate 2 in 60 ml of dichloromethane, was added dropwise over 75 minutes. The next day the stirring had stopped due to a gummy precipitate. After 40 hours, 150 cubic cm of crushed ice was added, and the mixture stirred to slowly digest the dark solid. Once the bulk of the gummy mass was digested, 20 g of sodium chloride was added, followed by 20 ml of hydrochloric acid.

After stirring an additional 20 minutes, the mixture was filtered and the filter cake was washed with 100 ml 1M hydrochloric acid, and then 100 ml of methylene chloride. The layers were separated, and the methylene chloride layer was washed with 200 ml of 10% potassium carbonate. The potassium carbonate wash was extracted with an additional 200 ml of methylene chloride. The methylene chloride layers were combined and evaporated in vacuo to give 14 grams of dark oil. The crude dark oil was taken up in 200 ml of methanol, 10 ml of 50% sodium hydroxide was added, and the mixture was stirred overnight. The resulting suspension was treated with 40 ml of water to provide a cloudy solution, which was treated with celite and filtered. The mix was evaporated in vacuo to provide a dry solid. The solid was dissolved 400 ml water, treated with carbon and filtered. The filtrate was neutralized to pH 6 with sulfuric acid, and the resulting solids collected by filtration, washed with water, and air dried to give intermediate 4 as the amino acid, 10.28 g.

| | % C | % H | % N | % H₂O |
|---|---|---|---|---|
| Found: | 62.21 | 6.32 | 3.96 | 5.28 |
| Calc.: | 62.24 | 6.09 | 4.03 | 5.19 |

Calculated for C₁₈H₁₉NO₅ × H₂O

### N-(5-tert-butyl-2-methoxyphenyl)-2-{4-[2-(morpholin-4-yl)ethoxy]naphthalen-1-yl}-2-oxoacetamide

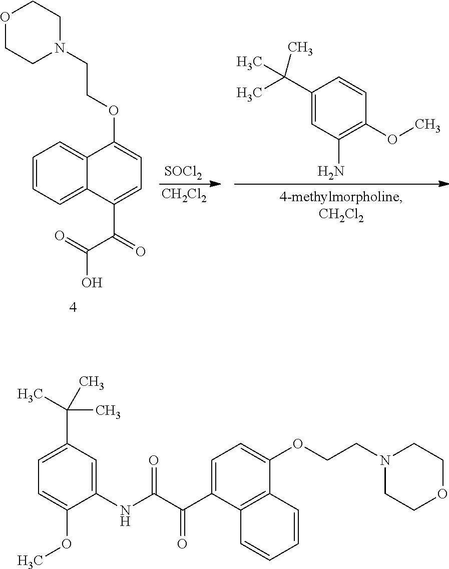

Intermediate 4 (7.80 grams, 0.022 moles) in a 250 ml RB flask with magnetic stirring bar was treated with 50 ml of dichloromethane. 5 ml of thionyl chloride (0.068 mole) was added slowly. With the addition of the first 0.5 ml, the mixture became thick, and an additional portion of methylene chloride (50 ml) was added to allow stirring. After the addition of the thionyl chloride, 0.5 ml of dimethylformamide was added. After stirring overnight at room temperature, the mix was placed under house vacuum (˜60 mm Hg) and evaporated until no further condensate collected in a −20 C trap, giving dry solids in the reaction flask. A solution of 4.03 g of 5-tert-butyl-o-anisidine in 6 ml dichloromethane was prepared. The reaction mixture was treated with 50 ml of methylene chloride to re-suspend the solids. The solution of 5-tert-butyl-o-anisidine was added slowly, followed by 4 ml of 4-methylmorpholine in 10 ml of dichloromethane. The mixture was then stirred 3 hours at room temperature. The mixture was the transferred to a 500 ml flask to provide a total of 250 ml of methylene chloride and 200 ml of 5% aqueous potassium carbonate. After thorough mixing, the layers were separated and the dark yellow organic layer was diluted with 100 ml of methanol and concentrated hot. As the volume reached 250 ml, an additional 100 ml of methanol was added. The hot concentration was continued, and an additional 60 ml of methanol was added as the volume was reduced to 300 ml. The hot concentration was continued, and when the vapor temperature of the distillate reached 57° C., the solids separated. Heating was terminated and the mixture was allowed to cool with stirring. After stirring over the weekend, the flask containing the suspension of solids was chilled in an ice water bath and stirred 40 min. The solids were then collected by filtration and air dried to yield 7.23 g of the product.

| | % H | % N | % H₂O |
|---|---|---|---|
| Found: | 70.71 | 5.72 | 0.44 |
| Calc.: | 70.74 | 5.69 | 0.37 |

Calculated for C₂₉H₃₅ClN₂O₅ × 0.2H₂O

### N-(5-tert-butyl-2-methoxyphenyl)-2-{4-[2-(morpholin-4-yl)ethoxy]naphthalen-1-yl}-2-oxoacetamide hydrochloride

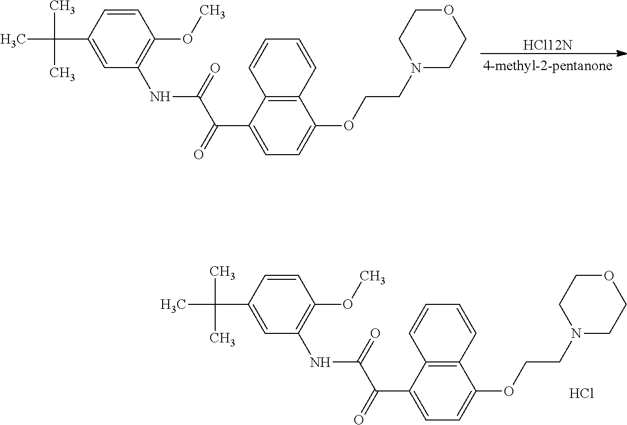

18.4 g of N-(5-tert-butyl-2-methoxyphenyl)-2-{4-[2-(morpholin-4-yl)ethoxy]naphthalen-1-yl}-2-oxoacetamide was placed in a 1 L flask with a 4 cm magnetic stirring bar. 370 ml of 4-methyl-2-pentanone was added, followed by 2 ml of water. The mixture was stirred and warmed until a clear yellow solution had formed, with a solution temperature of 84° C. 4.1 ml of hydrochloric acid was added dropwise over 2 minutes with stirring. Thick solids precipitated after the addition was complete. The mix was stirred and allowed to cool. After 4 hours at room temperature, the solids were collected by filtration, washed with 2×20 ml of 4-methyl-2-pentanone, and air dried to provide N-(5-tert-butyl-2-methoxyphenyl)-2-{-4-[2-(morpholin-4-yl)ethoxy]naphthalen-1-yl}-2-oxoacetamide hydrochloride, 18.96 g.

| | % C | % H | % N | % Cl |
|---|---|---|---|---|
| Found: | 63.95 | 6.94 | 5.15 | 6.42 |
| Calc.: | 63.90 | 6.84 | 5.14 | 6.50 |

Calculated for C₂₉H₃₄N₂O₅ × HCl

### Method C:

### Intermediate 6: 5,2-(naphthalen-1-yloxy)ethyl acetate

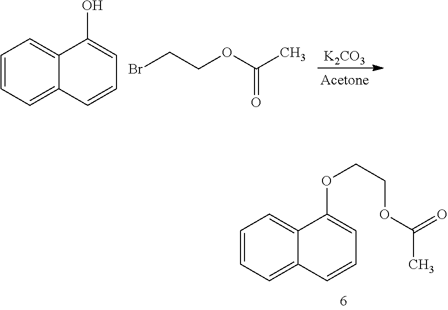

Materials:

| | mole wt | DEN | moles | eq | wt/vol used |
|---|---|---|---|---|---|
| 1-naphthol | 144.19 | | 0.144461 | 0.965 | 20.83 g |
| acetic acid 2-bromoethyl ester | 167 | 1.514 | 0.149701 | 1 | 25 g |
| Potassium carbonate anhydrous | 138 | | 0.288922 | 2 | 39.871 g |
| Tetrabutylammonium iodide | 369.38 | | 0.003357 | 0.022425 | 1.24 g |

In a 200 ml flask was placed acetic acid 2-bromoethyl ester, then 20.8 g of 1-naphthol, followed by 37.8 g of potassium carbonate. 100 ml of dry acetone was added, followed by 1.24 g tetra butyl ammonium iodide. The mixture was warmed to 50° C. After 48 hours, the mixture was filtered to remove solids. The solids were washed with acetone until the filtrate was nearly colorless. The combined filtrates were evaporated in vacuo. The residue was diluted with 300 ml of hexanes and vigorously stirred, then allowed to settle. The hexanes were evaporated in vacuo, providing 2-(naphthalen-1-yloxy)ethyl acetate, a yellow oil, 28.32 g. HPLC at 254 nM showed a new peak at 5.35 min 73% and 1-naphthol, 2.8%.

### Intermediate 7: 5,2-(naphthalen-1-yloxy)ethyl acetate

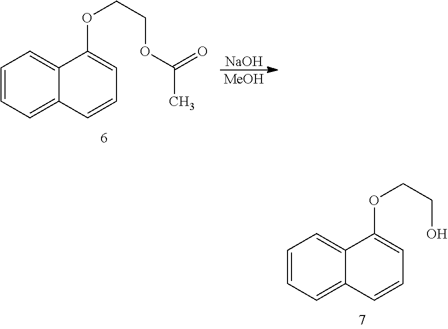

14 g of 2-(naphthalen-1-yloxy)ethyl acetate was diluted with 100 ml of methanol and 10 ml of 50% sodium hydroxide was added. The mixture was stirred over night. The mixture was then poured onto ice, 160 g, and diluted with water to 400 ml. The mixture was washed with 80 ml of methylene chloride, and 100 ml of hexanes. The extracts were combined and evaporated to provide 10.8 g of a dark oil. TLC one new spot (RF 0.25) with methylene chloride on silica. HPLC showed new peak at 4.09 min, 71%.

### Intermediate 8: 1-(2-chloroethoxy)naphthalene

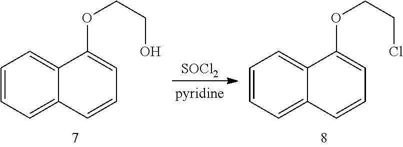

A flask containing 10.8 g of intermediate 7 was treated with 60 ml of toluene and the solution transferred to a 250 ml flask with a stirring bar. The flask previously containing intermediate 7 was washed with an additional portion of 10 ml of toluene, which was then also added to the 250 ml flask. 4.64 ml of pyridine was added and the flask was placed in an oil bath with stirring. 4.16 ml of thionyl chloride was added over two minutes at an oil bath temperature of 60° C. The mix was then warmed to an oil bath temperature of 120° C. The mixture refluxed gently, and evolved gasses. Gas evolution slowed after 20 minutes. The bath was warmed to 130° C. TLC after 10 min showed that intermediate 7 was consumed and a new spot (RF 0.9) had appeared (methylene chloride on silica). The reaction mixture was poured onto 100 g ice in 10 ml hydrochloric acid and extracted with 50 ml of hexanes. The organic layer was evaporated to give 11.9 g of 1-(2-chloroethoxy)naphthalene as a dark oil. HPLC showed a new peak at 5.87 min, 62% at 254 nM.

Alternatively, intermediate 8 may be prepared by the following route: A sample of 1-naphthol, 17.03 g, was placed in a 250 ml flask, potassium carbonate, 45 g, was added, followed by 2-butanone, 60 ml, and 1-bromo-2-chloroethane, 14 ml. The flask was placed in an oil bath that was warmed to 90° C., and the mixture was vigorously stirred. After five hours the mix was filtered and the solids were washed with hexanes, 3×40 ml. TLC of the filtrate showed strong product spot (RF 0.62) and a weak spot for naphthol (RF 0.16) with 1:1 DCM/Hexanes on silica. The filtrate was washed with 1M Sodium hydroxide, 2×125 ml. The remaining organic layer showed only the new spot (RF 0.62), and no naphthol. The organic layer was evaporated in vacuo to provide intermediate 8, 16.14 g. HPLC showed 93% pure UV 254 nM.

### Intermediate 9: ethyl [4-(2-chloroethoxy)naphthalen-1-yl]oxoacetate

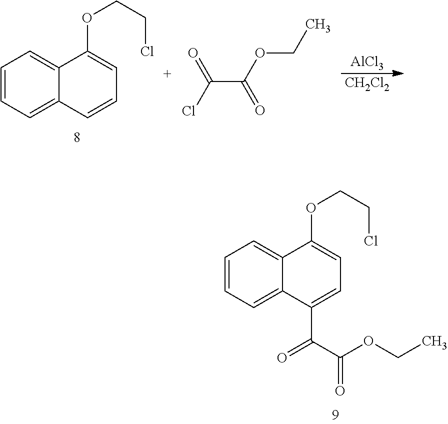

9.99 g of aluminum chloride was placed in a 250 ml flask with a stirring bar. 100 ml of dry methylene chloride was added, followed by 7.0 ml of ethyl chlorooxoacetate. The resulting nearly clear solution was treated with 11.9 g of intermediate 8 in small portions at such a rate that the solution refluxed gently. After the addition, the mixture was stirred 20 minutes, then poured on to 100 g of crushed ice, and stirred well for 15 minutes. The layers were separated, and the aqueous layer was washed with 40 ml of methylene chloride. The combined methylene chloride layers were diluted with 150 ml of 95% ethanol and chilled to −20° C. overnight. The resulting solids were collected by filtration, washed with 25 ml ethanol, and air dried to give 8.77 g of ethyl [4-(2-chloroethoxy)naphthalen-1-yl]oxoacetate as an off white solid. HPLC showed new peak 5.859 minutes (99.7%). Concentration of the heated filtrate to 100 ml, and chilling provided a second crop of ethyl [4-(2-chloroethoxy)naphthalen-1-yl]oxoacetate as a tan solid (3.23 g). HPLC of the second crop showed 91.2% purity. ¹H NMR (500 mHz, CDCl₃): 9.20, 1H, J=8.7, d; 8.39, 1H, J=8.1, d; 7.97, 1H, J=8.3, d; 7.72, 1H, dd; 7.60, 1H, dd; 6.82, 1H, J=8.3, dd; 4.50, 2H, J=5.7, t; 4.47, 2H, J=7.2, q; 4.00, 2H, J=5.7, t; 1.44, 3H, J=7.2, t.

### Intermediate 10: [4-(2-chloroethoxy)naphthalen-1-yl]oxoacetic acid

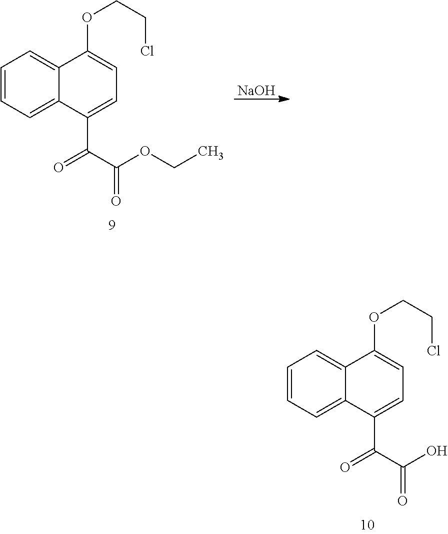

16.5 g of 50% sodium hydroxide was added to 75 ml of 95% ethanol and stirred until clear. The mixture was diluted with 95% ethanol to 103 ml to give 2 M sodium hydroxide in ethanol. 3.08 g of intermediate 10 was dissolved in 20 ml of tetrahydrofuran. A sample of the 2M sodium hydroxide in ethanol prepared above, 10 ml, was measured out. The 2M sodium hydroxide was added in small portions and the mixture was treated with 30 ml of tetrahydrofuran followed by 80 ml of 95% ethanol to maintain stirring. After 1.5 hours, the solids were collected by filtration, washed with 2×12 ml tetrahydrofuran and air dried to give 3.06 g of a waxy solid. The waxy solids were placed in water, 100 ml, and stirred, giving a suspension. The suspension was acidified with 10 ml of 1M sulfuric acid and extracted with 30 ml of methylene chloride. The aqueous layer was washed with an additional 20 ml of methylene chloride. The combined methylene chloride were concentrated hot to 20 ml, and a distillate temperature of 40° C., then diluted slowly with 30 ml of hexanes. The mix was cooled with stirring. Filtration provided 1.91 g. of intermediate 10 as a light tan solid. MS negative ion (M−1)=277.6. HPLC showed a new peak (99.7%) at 4.168 min. ¹H NMR (500 mHz, CDCl₃): 9.03, 1H, J=8.6, d; 8.80, 1H, J=8.4, d; 8.43, 1H, J=8.4, d; 7.74, 1H, dd; 7.62, 1H, dd; 6.86, 1H, J=8.6, d; 4.53, J=5.6, t; 4.01, J=5.6, t

Alternatively, intermediate 10 was prepared as follows: 0.309 g of intermediate 9 and 2 g of dipropylene glycol were briefly warmed with a heat gun to provide a solution. 0.7 ml of 2M NaOH was added over 5 min, followed by 1 ml of water. The mixture was stirred 2 minutes and 3 ml of water was added. The mixture was then treated with 2 ml of 1M sulfuric acid, diluted with water to 30 ml and chilled on ice. Filtration of the cloudily mixture provided no solids. The mixture was diluted with water to 100 ml and warmed on a hot plate, giving a suspension of solids. The mixture was stirred and chilled in ice, then filtered and the solids washed with water and air dried to give 0.241 g of intermediate 10, which was identical to the material as provided above by HPLC.

### Intermediate 11: N-(5-tert-butyl-2-methoxyphenyl)-2-[4-(2-chloroethoxy)naphthalen-1-yl]-2-oxoacetamide

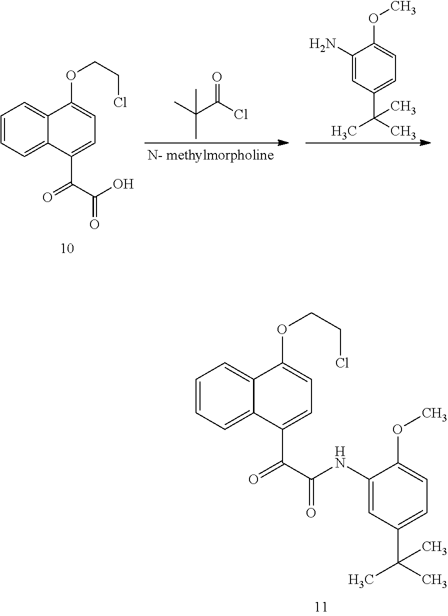

1.85 g of intermediate 10 was placed in a 100 ml flask with a stirring bar. 20 ml of dry methylene chloride was added, and the mixture was placed under a septa and a balloon. 0.97 ml of N-methylmorpholine was added giving a clear light yellow solution. 0.84 ml of trimethylacetyl chloride was added, and a precipitate formed after 2 minutes. After 10 minutes, 5-tert-Butyl-o-anisidine (1.22 g, in methylene chloride) was added, followed by 1 ml of N-methylmorpholine and the mixture was stirred at room temperature. After 3 hours, the mixture was diluted with 20 ml of methylene chloride and washed with 50 ml of 10% potassium carbonate. The organic layer was concentrated to 30 ml and diluted with 30 ml of hexanes, then chilled to −20° C. overnight. The resulting solids were collected by filtration and washed with hexanes, 10 ml, then air dried to give 2.56 g N-(5-tert-butyl-2-methoxyphenyl)-2-[4-(2-chloroethoxy)naphthalen-1-yl]-2-oxoacetamide as a yellow solid. MS negative ion (M−1)=438, positive ion (M+1)=440. ¹H NMR (500 mHz, CDCl₃): 9.68, 1H, s; 8.82, 1H, J=8.5, d; 8.63, 1H, J=2.4, d; 8.6, 1H, J=8.4, d; 8.41, 1H, J1=0.8, J2=8.4, dd; 7.68, 1H, m; 7.58, 1H, m; 7.15, 1H, J1=2.4, J2=8.6, dd; 6.88, 1H, J=8.6, d; 6.86, 1H, J=8.4, d; 4.51, 2H, J=5.7, t; 4.01, 2H, J=5.7, t; 3.93, 3H, s; 1.35, 9H, s.

### Intermediate 5: N-(5-tert-butyl-2-methoxyphenyl)-2-{4-[2-(morpholin-4-yl)ethoxy]naphthalen-1-yl}-2-oxoacetamide

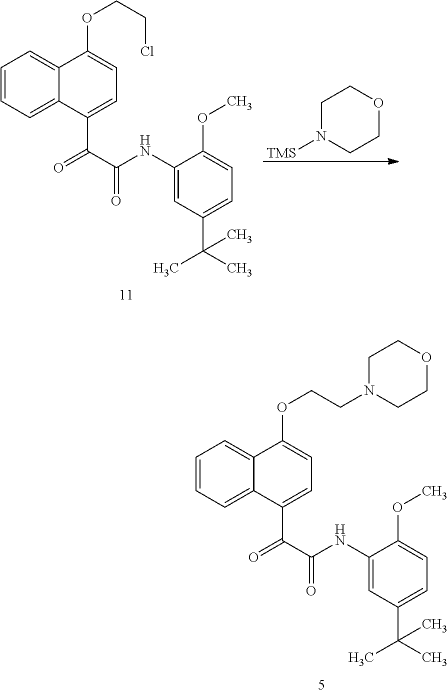

2.228 g of intermediate 11 was placed in a 75 ml heavy wall Teflon screw top flask. 0.248 g of sodium iodide was added, followed by 20 ml of 1-methoxy-2-propanol and 2.8 ml of trimethylsiliylmorpholine. The flask was closed and heated in a 90° C. oil bath for 42 hours. The flask was then allowed to cool, the contents transferred to a 250 ml flask, and the heavy wall flask was washed with methanol (2×3 ml) an added to the 250 ml flask. The solution was diluted with water to 100 ml, stirred 20 minutes, and the solids collected by filtration. The solids were washed with 20 ml water and air dried, providing 2.305 g of N-(5-tert-butyl-2-methoxyphenyl)-2-{-4-[2-(morpholin-4-yl)ethoxy]naphthalen-1-yl}-2-oxoacetamide (intermediate 5). HPLC retention time and UV, showed the sample to contain 71% of intermediate 5 and 27.4% remaining intermediate 11.

Alternatively, compound 5 was prepared from intermediate 11 as follows: 3 g of intermediate 11 was placed in a 15 ml screw top vial with stirring bar, and 6 ml of morpoline was added. The mixture was warmed in a 100° C. oil bath for three hours. The mix was then allowed to cool to room temperature. A solid paste was removed from the tube, diluted with methanol to 100 ml, and warmed to reflux, and further diluted with water, 25 ml. At no time did the solids completely dissolve. The mixture was then chilled with ice and solids were collected by filtration, washed with water and air dried to provide compound 5 (94.99% by HPLC, 25 to 95% acetonitrile, 0.05% TFA).

### N-(5-tert-butyl-2-methoxyphenyl)-2-{4-[2-(morpholin-4-yl)ethoxy]naphthalen-1-yl}-2-oxoacetamide hydrochloride

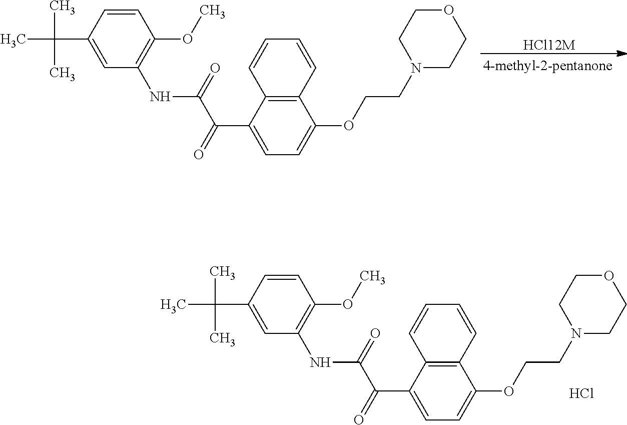

N-(5-tert-butyl-2-methoxyphenyl)-2-{4-[2-(morpholin-4-yl)ethoxy]naphthalen-1-yl}-2-oxoacetamide hydrochloride was prepared as shown above in Method B to yield 1.37 g (97.46% pure by HPLC).

Table 1 lists compounds provided herein that may be prepared using the methods of Example 1, or methods previously disclosed in the art.

TABLE 1

| No. | Structure | Name |
|---|---|---|
| 1 | 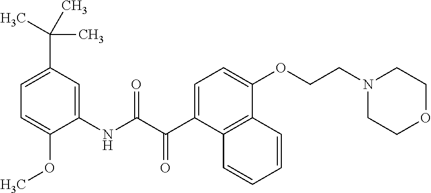 | N-(5-tert-butyl-2-methoxyphenyl)-2- (4-(2-morpholinoethoxy)naphthalen-1- yl)-2-oxoacetamide |
| 2 | 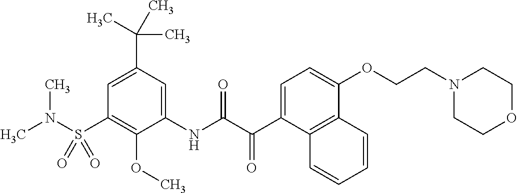 | N-(5-tert-butyl-3-(N,N- dimethylsulfamoyl)-2- methoxyphenyl)-2-(4-(2- morpholinoethoxy)naphthalen-1-yl)-2- oxoacetamide |
| 3 | 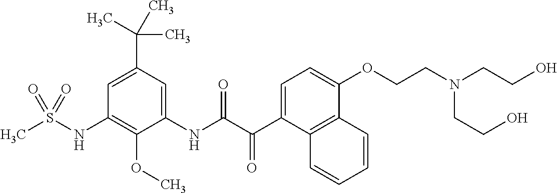 | 2-(4-(2-(bis(2- hydroxyethyl)amino)ethoxy)- naphthalen-1-yl)-N-(5-tert-butyl-2- methoxy-3- (methylsulfonamido)phenyl)-2- oxoacetamide |
| 4 | 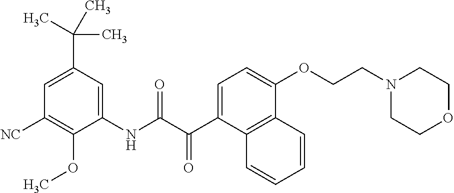 | N-(5-tert-butyl-3-cyano-2- methoxyphenyl)-2-(4-(2- morpholinoethoxy)naphthalen-1-yl)-2- oxoacetamide |
| 5 | 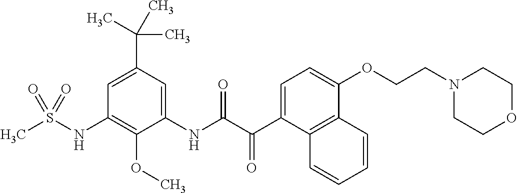 | N-(5-tert-butyl-2-methoxy-3- (methylsulfonamido)phenyl)-2-(4-(2- morpholinoethoxy)naphthalen-1-yl)-2- oxoacetamide |
| 6 | 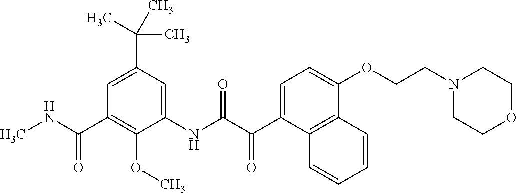 | 5-tert-butyl-2-methoxy-N-methyl-3-(2- (4-(2-morpholinoethoxy)naphthalen-1- yl)-2-oxoacetamido)benzamide |
| 7 | 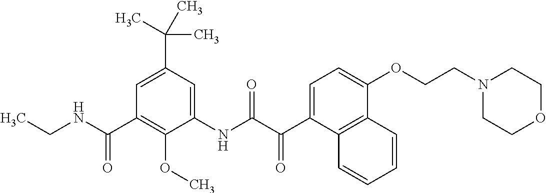 | 5-tert-butyl-N-ethyl-2-methoxy-3-(2- (4-(2-morpholinoethoxy)naphthalen-1- yl)-2-oxoacetamido)benzamide |
| 8 | 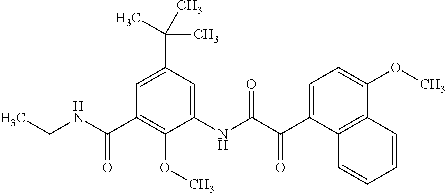 | 5-tert-butyl-N-ethyl-2-methoxy-3-(2- (4-methoxynaphthalen-1-yl)-2- oxoacetamido)benzamide |
| 9 | 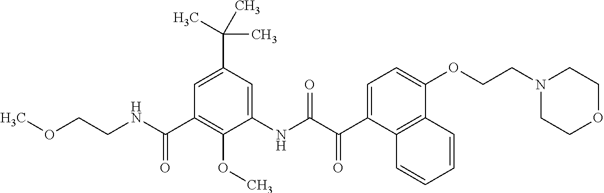 | 5-tert-butyl-2-methoxy-N-(2- methoxyethyl)-3-(2-(4-(2- morpholinoethoxy)naphthalen-1-yl)-2- oxoacetamido)benzamide |
| 10 | 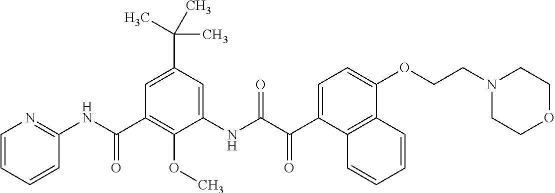 | 5-tert-butyl-2-methoxy-3-(2-(4-(2- morpholinoethoxy)naphthalen-1-yl)-2- oxoacetamido)-N-(pyridin-2- yl)benzamide |
| 11 | 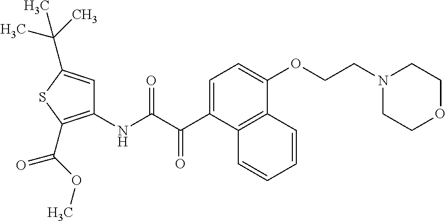 | methyl 5-tert-butyl-3-(2-(4-(2- morpholinoethoxy)naphthalen-1-yl)-2- oxoacetamido)thiophene-2-carboxylate |
| 12 | 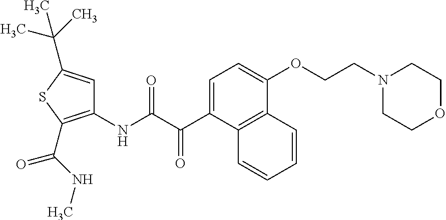 | 5-tert-butyl-N-methyl-3-(2-(4-(2- morpholinoethoxy)naphthalen-1-yl)-2- oxoacetamido)thiophene-2- carboxamide |
| 13 | 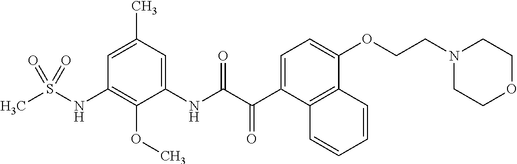 | N-(2-methoxy-5-methyl-3- (methylsulfonamido)phenyl)-2-(4-(2- morpholinoethoxy)naphthalen-1-yl)-2- oxoacetamide |
| 14 | 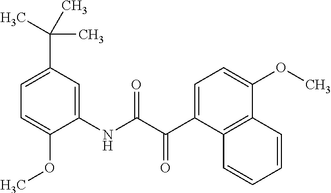 | N-(5-tert-butyl-2-methoxyphenyl)-2- (4-methoxynaphthalen-1-yl)-2- oxoacetamide |
| 15 | 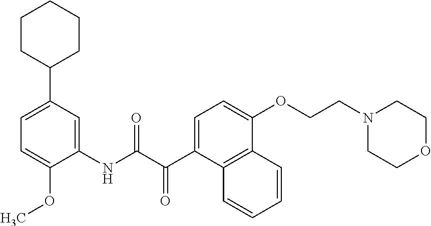 | N-(5-cyclohexyl-2-methoxyphenyl)-2- (4-(2-morpholinoethoxy)naphthalen-1- yl)-2-oxoacetamide |
| 16 | 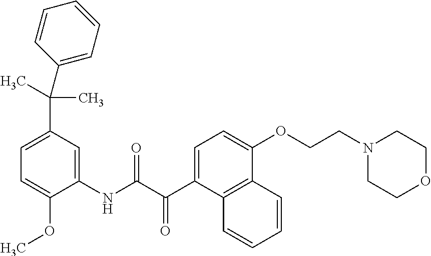 | N-(2-methoxy-5-(2-phenylpropan-2- yl)phenyl)-2-(4-(2- morpholinoethoxy)naphthalen-1-yl)-2- oxoacetamide |
| 17 | 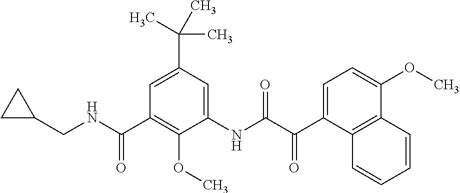 | 5-tert-butyl-N-(cyclopropylmethyl)-2- methoxy-3-(2-(4-methoxynaphthalen- 1-yl)-2-oxoacetamido)benzamide |
| 18 | 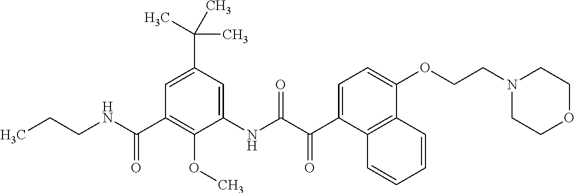 | 5-tert-butyl-2-methoxy-3-(2-(4-(2- morpholinoethoxy)naphthalen-1-yl)-2- oxoacetamido)-N-propylbenzamide |
| 19 | 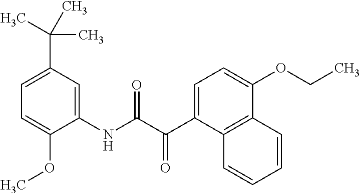 | N-(5-tert-butyl-2-methoxyphenyl)-2- (4-ethoxynaphthalen-1-yl)-2- oxoacetamide |
| 20 | 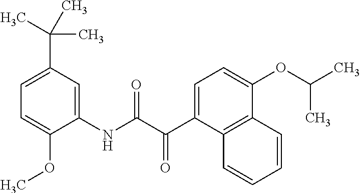 | N-(5-tert-butyl-2-methoxyphenyl)-2- oxo-2-[4-(propan-2-yloxy)naphthalen- 1-yl]acetamide |

### Example 3

### Preparation of 5-tert-butyl-2-methoxy-3-(2-(4-(6-(methylamino)pyridin-3-yl)naphthalen-1-yl)-2-oxoacetamido)benzamide

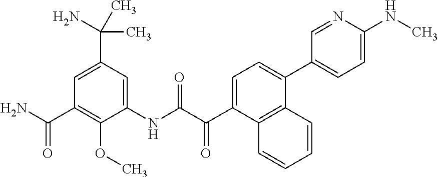

### Intermediate 12: 5-tert-Butyl-2-methoxy-3-nitrobenzoic acid

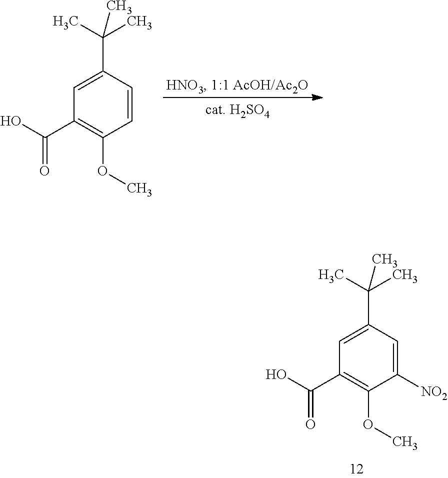

To a solution of 5.5 g (26 mmol) of 5-t-butyl-2-methoxybenzoic acid in 30 ml acetic acid and 30 ml acetic anhydride was added catalytic amount (ca. 5 drops) concentrated sulfuric acid, followed by drop-wise addition of fuming nitric acid (72 mmol) at 0° C. The resulting reaction was stirred at r.t. for overnight. After reaction, the mixture was poured in ca. 1.2 L ice-water. White precipitate was formed which was filtered and washed with water. 5.98 g of a white solid was obtained (yield: 91%).

### Intermediate 13: 5-tert-butyl-2-methoxy-3-nitrobenzamide

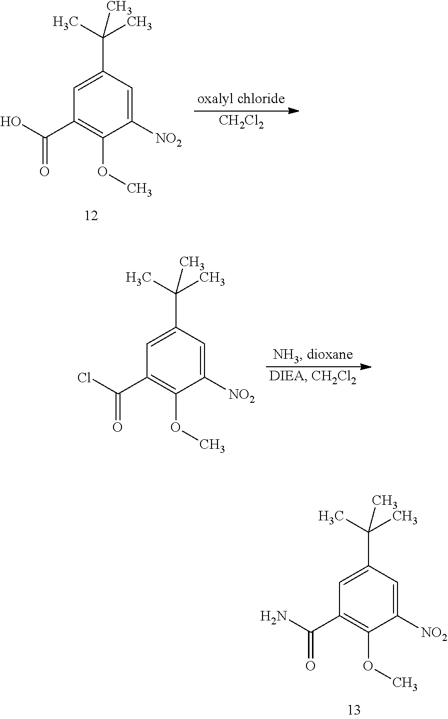

To a solution of intermediate 12 (1.985 g, 7.84 mmol) in 50 ml of CH₂Cl₂ was added oxalyl chloride (3.5 ml, 5 eq.). After stirring at room temperature for 2 hours the reaction mixture was evaporated to dryness. The resulting acid chloride was dissolved in 40 ml of CH₂Cl₂, followed by addition of diisopropylethylamine (4 ml, 3 eq.) and a solution of 0.5 N ammonia in dioxane (47 ml, 3 eq.). Stirring was continued for overnight at room temperature and CH₂Cl₂ was added to the reaction mixture. After aqueous sodium bicarbonate work-up, column purification was done on ISCO Optix (3×40 g silica gel column) using 0-40% B (10% methanol in CH₂Cl₂). 1.796 g product was isolated as pale yellow solid (yield: 90.8%).

### Intermediate 14: 2-(4-bromonaphthalen-1-yl)-2-oxoacetic acid

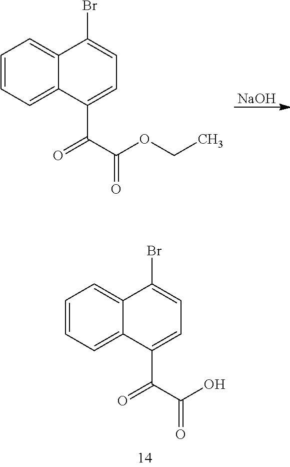

(4-Bromo-naphthalen-1-yl)-oxo-acetic acid methyl ester is prepared as in U.S. Patent Publication No. 2005/0107399. The resulting (4-Bromo-naphthalen-1-yl)-oxo-acetic acid methyl ester, 30.7 g, is treated with dipropylene gycol, 150 g, and warmed to 50° C. A 2M solution of 50% Sodium hydroxide in 95% ethanol, 60 ml, is added to the mixture. The mixture is stirred at 50° C. for 15 minutes, and then 1 M sulphuric acid, 70 ml is added. The mixture is diluted with water to 1.5 l, and stirred and allowed to cool. The solids collected by filtration to provide intermediate 14.

### Intermediate 15: 3-(2-(4-bromonaphthalen-1-yl)-2-oxoacetamido)-5-tert-butyl-2-methoxybenzamide

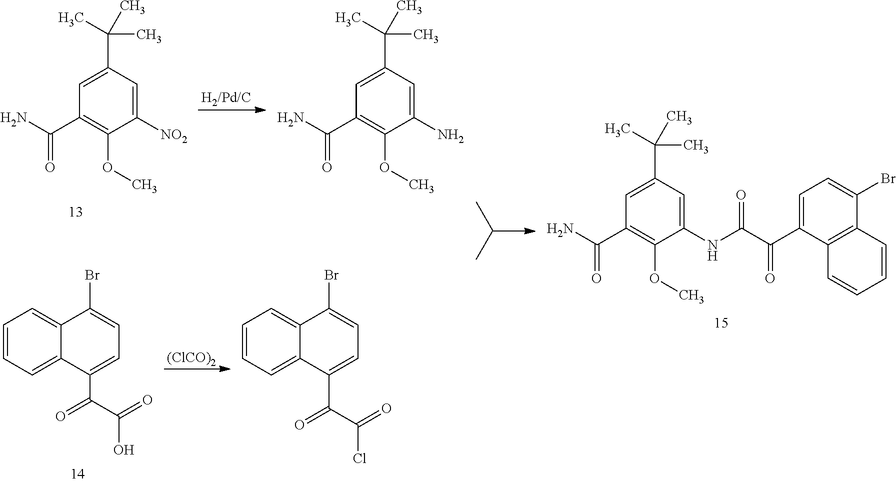

Intermediate 13 (1.031 g, 4.07 mmol) was reduced with Pd/C and H₂ in 40 ml methanol at room temperature for 3 hours. After filtration, methanol was removed and the crude reduced compound was dissolved in 25 ml of CH₂Cl₂. 2.6 ml (3 eq.) diisopropylethylamine was added. In another flask, intermediate 14 (1.36 g, 1.2 eq.) was dissolved in 30 ml of CH₂Cl₂ and 2.2 ml (5 eq.) of oxalyl chloride was added followed by addition of a catalytic amount of DMF. The reaction mixture was stirred at room temperature for 2 hours and the solvent was evaporated. The resulting crude acid chloride was dissolved in 15 ml of CH₂Cl₂, and the above amine solution was added. The mixture was stirred for 40 hours at room temperature and CH₂Cl₂ was added. Aqueous sodium bicarbonate work-up followed by ISCO column purification (3×40 g silica column, 0-40% B 50% ethyl acetate in CH₂Cl₂) gave 1.08 g of the product as a yellow solid (yield: 54.9%).

### Intermediate 16: N-methyl-5-(tributylstannyl)pyridin-2-amine

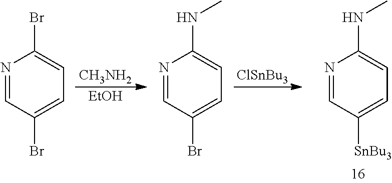

2,5-Dibromopyridine (6 g, 25 mmol) in 40 ml 33% CH₃NH₂ in ethanol (13 eq.) was heated at 80° C. for 60 hours. After evaporation the solid residue was suspended in CH₂Cl₂, extracted 3× with 1 N HCl. The combined aqueous phase was neutralized with 2 N NaOH (pH-10-11), and then back extracted 3× with CH₂Cl₂. The combined CH₂Cl₂ phase was washed with water and dried over sodium sulfate. Concentration gave 4.457 g of 5-bromo-N-methylpyridin-2-amine as off-white solid. (yield: 95.3%).

5-bromo-N-methylpyridin-2-amine (2,805 g, 15 mmol) in 150 ml of dry THF under N₂ atmosphere was cooled to −78° C. 19.4 ml of 1.7 M t-BuLi (2.2 eq.) in heptane was added dropwise. Lithiation was continued at the same temperature for 30 min before addition of a solution of tri-n-butyltin chloride (8.9 ml, 2.2 eq.) in 15 ml dry THF. The reaction was stirred at −78° C. for 2 hours and quenched by 5% acetic acid in THF. The temperature was allowed to rise slowly to room temperature. After removal of solvent by evaporation, the residue was dissolved in ethyl acetate/water and worked-up with aqueous NaHCO₃. The organic phase was dried over sodium sulfate, concentrated and subjected to ISCO column (10×40 g column) purification first with CH₂Cl₂ (30 min) then with 1:3 ethyl acetate/hexane 1:1 ethyl acetate/hexane (30 min) to give 2.44 g of product as white soft solid (yield: 41%).

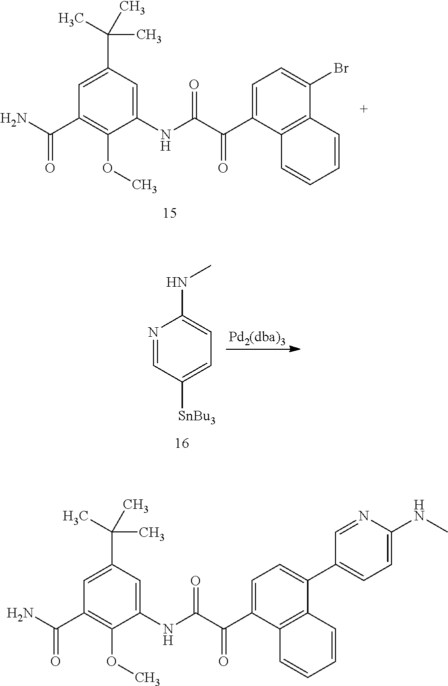

A solution of intermediate 15 (1.633 g, 3.378 mmol), intermediate 16 (1.61 g, 4.05 mmol, 1.2 eq.) and Pd catalyst (490 mg, 14% mol) in 10 ml dry toluene and 30 ml dry dioxane was heated at 110° C. for 24 hours under N₂. DMF was added after evaporation. The red solution was subjected to HPLC reverse phase purification after filtration (68 injections, method: 25-95% acetonitrile 12 min). The fractions containing product were collected. The organic solvent was removed under reduced pressure. Solid sodium bicarbonate was added to the suspension followed by addition of ethyl acetate. Organic phase was separated, and the aqueous was extracted one more time with ethyl acetate. The combined organic phase was dried over sodium sulfate. Concentration gave 727 mg of 5-tert-butyl-2-methoxy-3-(2-(4-(6-(methylamino)pyridin-3-yl)naphthalen-1-yl)-2-oxoacetamido)benzamide (42% yield).

Hydrochloride Salt: 5-tert-butyl-2-methoxy-3-(2-(4-(6-(methylamino)pyridin-3-yl)naphthalen-1-yl)-2-oxoacetamido)benzamide was dissolved in CH₂Cl₂, and 2 ml of 4 M HCl in dioxane was added. CH₂Cl₂ was evaporated and dry dioxane was added. The suspension was frozen and lyophilized to give 755 mg of the salt as a fine orange powder.

### Example 4

### HCV2a Chimeric Luciferase Virus Entry Assay

The instant example demonstrates that compounds provided herein have activity against HCV infection. In addition, the instant example demonstrates that compounds of the invention show advantageous efficacy, or cytotoxicity, or both when compared to cyclosporin A.

Preparation of cells: Naïve Huh7 cells were grown in cell culture media composed of Dulbecco's modified Eagle's medium (Gibco BRL) supplemented with 10% fetal bovine serum (Sigma), 1× non-essential amino acids (100× for MEM, IrvineScientific), 1 mM of sodium pyruvate (GIBCO), and 1× penicillin/streptomycin (Invitrogen). Cell lines were passaged once or twice per week. Subconfluent cells were washed once with PBS, lifted by trypsinization, and counted manually using a hemocytometer under a microscope. 5000 Huh7 cells were plated in each well of a 96-well plate. The plates were stored in an incubator (37° C., 5.0% of CO₂) until ready for use on the next day.

Preparation of HCV2a chimeric infectious virus: HCV 2a chimera genome (HCV2aCh) was constructed by combining the Core-NS2 region of the J6 genome with NS2-NS5B of the JFH1 genome at the region between the first and second putative transmembrane domains of NS2 (Jc1 crossover; Pietschmann et al., 2006, PNAS). A reporter version of this virus was made by inserting Renilla luciferase (Luc), FMDV 2A protease, and ubiquitin monomer sequence between 5′NTR of HCV2a and the open reading frame of HCV2a core protein (HCV2aChLuc). The N-terminus of Luc is fused to the 19th residue of the HCV core protein that is essential for HCV IRES function.

Viral RNA was transcribed in vitro using T7 Megascript kit (Ambion). DNase was added at the end of the reaction to remove the template DNA and then column purified (Qiagen, RNeasy mini kit). 5-10 μg of in vitro transcribed RNA was electroporated into Huh7 cells. HCV2aChLuc virus was harvested by collecting supernatants everyday post-transfection. The infectivity of the harvested viral supernatants was checked by luciferase assay (Renilla Luciferase Assay System, Promega) and/or limiting dilution assay by TCID₅₀ method (Lindenbach, B. D. 2009, Methods Mol. Biol.). Virus-containing supernatants were aliquoted to 50 ml conical tubes and stored at −80° C. until use.

Preparation of compound: 20 mM stock solution (100% DMSO) of the test compound was serially diluted in cell culture media containing a final concentration of 0.1% of DMSO to obtain the specific compound concentrations. In detail, 3 μl of the 20 mM stock was added to 200 μl of cell culture media (0.1% DMSO) giving a concentration of 300 μM. Again, 100 μl of 300 μM was serially diluted (1:3 dilution, 7 times) with cell culture media (0.1% of DMSO) to make the compound solution of the final concentration of 300 nM of the test compound. 300 nM was then further serially diluted (1:3) seven times repeatedly to prepare the compound solutions for use in the assay with the final concentrations of 100, 30, 10, 3, 1, 0.3, and 0.1 nM.

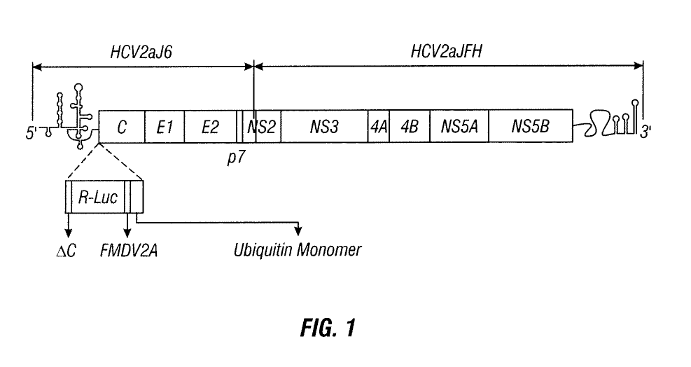

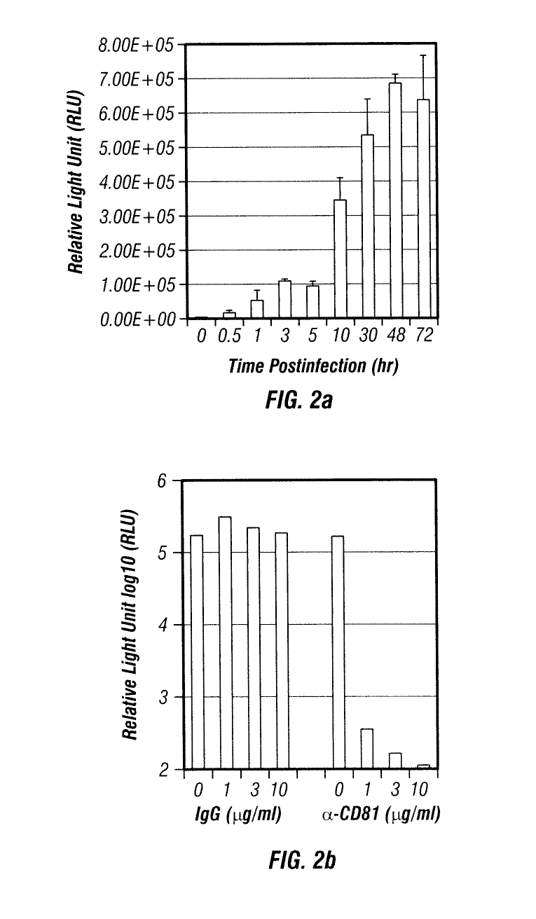

Results: Compound 1 and Compound 5 were assessed for the ability to inhibit HCV viral infection in a cell-based assay using infectious genotype 2a virus carrying a Renilla luciferase gene (HCV2aChLuc) (FIG. 1). Viral infectivity was validated by Taqman analysis, immunofluorescence, and western blotting as well as luciferase assay. Huh-7 cells were infected with HCV2aChLuc virus for 0, 0.5, 1, 3, 5, 10, 30, 48, 72 hr. At each time point, cells were harvested and luciferase activity was determined by Renilla Luciferase Assay (Promega). Up to 5 hrs after infection, only slow increase in the luciferase activity was observed, however, it was followed by a logarithmic rise luciferase activity between 5 and 30 hrs (FIG. 2a). Addition of Anti-CD81 Ab completely abolished luciferase activity, indicating that HCV2aChLuc viral infection is CD81-dependent. (FIG. 2b).

### Example 3

### In Vitro HCVcc Entry Assay (Validation of Anti-HCV Activity)

100 μl of serially diluted compound solutions (100, 30, 10, 3, 1, 0.3, and 0.1 nM) was added to the cells in a 96-well plate. Subsequently, 100 μl (TCID₅₀/ml: ˜7.0E+03) of HCV2a virus was added to the cells (MOI ˜0.1). The co-incubation of cells with compound and virus were maintained for 72 hrs in a cell culture incubator (37° C., 5.0% of CO₂). 72 hrs postinfection, virus and compound were removed from the cells. Cells were washed twice with PBS prior to lysis with 100 μl passive lysis buffer (Promega) per well. Cells were lysed at room temperature on a shaker for 15-20 minutes. 50 μl of the cell lysate was transferred to a new 96-well white plate (Costar) and 100 μl of substrate (Renilla Luciferase Assay System, Promega) was added to the cell lysates and immediately used to measure luciferase levels in the luminometer plate reader (Veritas-Turner Biosystems). Data was processed using MS Excel and GraphPad Prism.

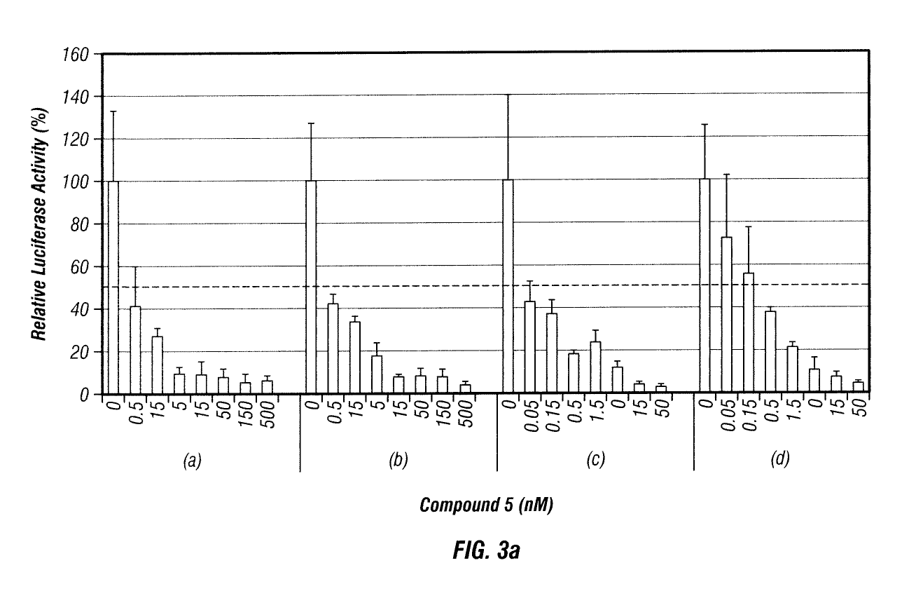

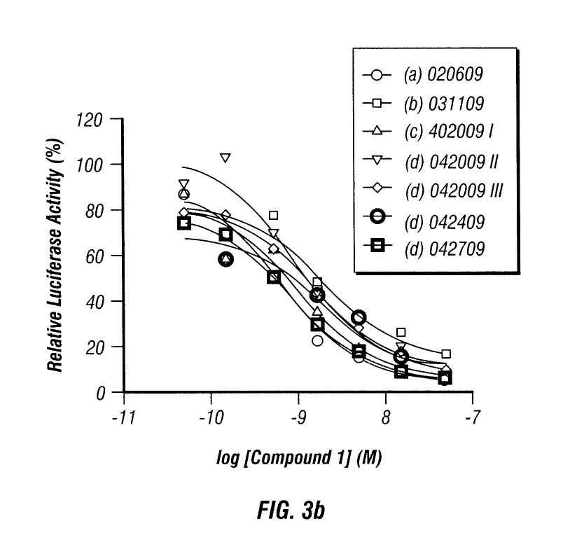

Results: To test the anti-HCV activities of Compound 1 and Compound 5, Huh7 cells were co-incubated with HCV2aChRluc virus in the presence of various amounts of Compound 1 and Compound 5 for 3 days. The assay was performed repeatedly (n>30), and a dose-dependent inhibition was consistently observed. Representative data are presented in FIG. 3. The observed IC₅₀ for Compound 1 was 0.2 to 2.0 nM. IC₉₀ was achieved at between 5.0 and 50.0 nM. The estimated EC₅₀ of each experiment was described in Table 2 below. The observed IC₅₀ for Compound 5 was 0.05 to 0.5 nM. IC₉₀ was achieved at between 5.0 and 15.0 nM.

TABLE 2
IC₅₀ of Compound 1 in HCV2aChLuc viral infection in vitro.
IC50 results for compound 1

| | | IC50 (95% Confidence |
|---|---|---|
| Date of Exp | IC50 (best fit value) | Intervals) |
| 020609 | 0.663 nM | 0.216~2.033 nM |
| 031109 | 1.906 nM | 0.594~6.118 nM |
| 042009 a | 1.021 nM | 0.263~3.972 nM |
| 042009 b | 0.907 nM | 0.281~2.924 nM |
| 042009 c | 1.598 nM | 1.122~2.276 nM |
| 042409 | 1.997 nM | 0.419~9.507 nM |
| 042709 | 0.798 nM | 0.549~1.161 nM |

Table 3 provides IC₅₀ data for additional compounds tested according to the method of Example 3. IC₅₀ values are provided as activity class “A” for an IC₅₀ less than or equal to 5 nM. Activity class “B” represents an IC₅₀ greater than 5 nM but less than 500 nM. Activity class “C” represents an IC₅₀ of 500 nM or greater.

TABLE 3

| No. | Structure | Activity class |
|---|---|---|
| 1 | 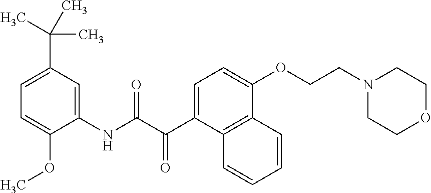 | A |
| 2 | 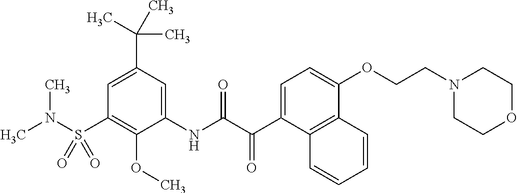 | A |
| 3 | 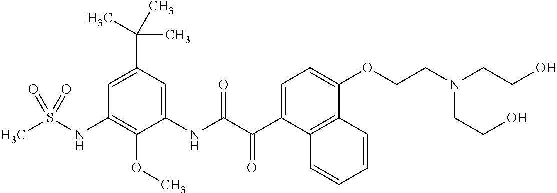 | A |
| 4 | 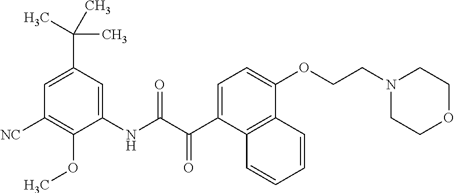 | A |
| 5 | 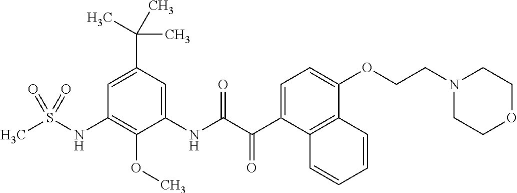 | A |
| 6 | 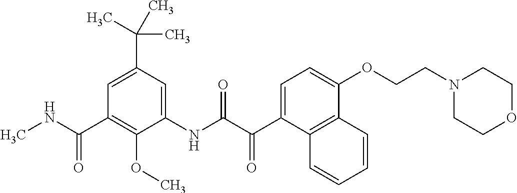 | A |
| 7 | 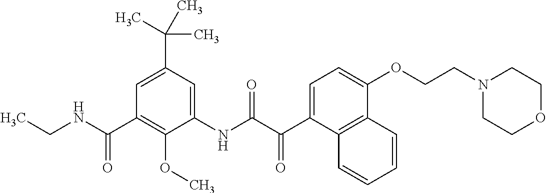 | A |
| 8 | 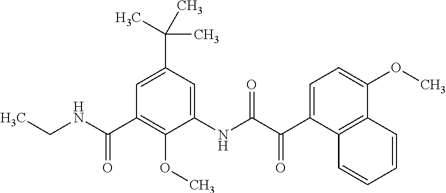 | A |
| 9 | 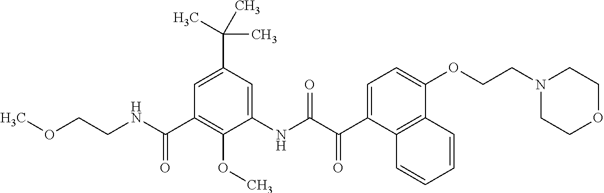 | A |
| 10 | 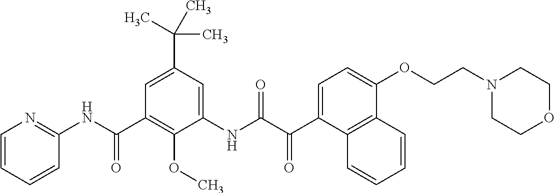 | A |
| 11 | 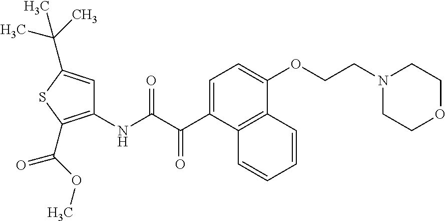 | A |
| 12 | 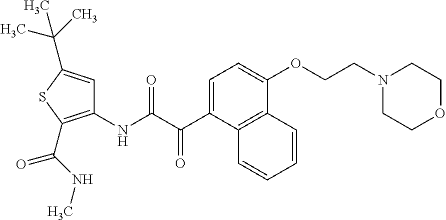 | A |
| 13 | 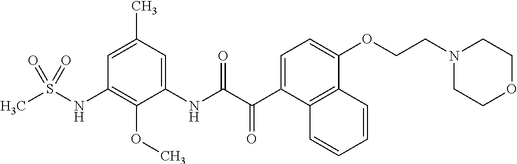 | B |
| 14 | 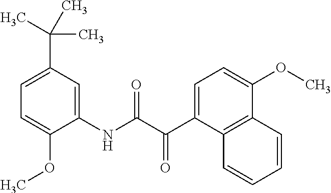 | A |
| 15 | 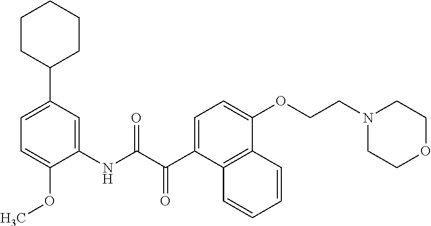 | A |
| 16 | 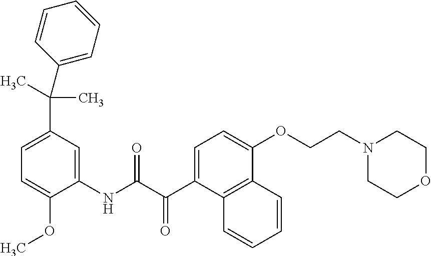 | A |
| 17 | 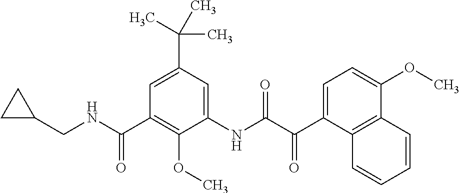 | A |
| 18 | 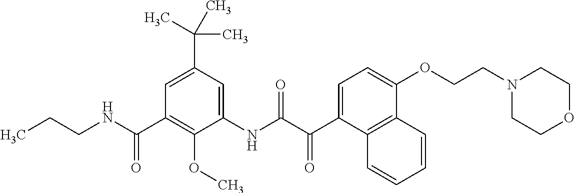 | A |
| 19 | 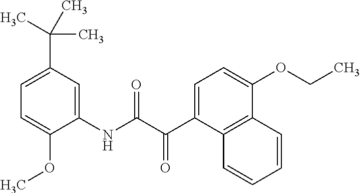 | A |
| 20 | 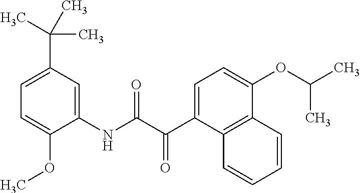 | A |
| 21 | 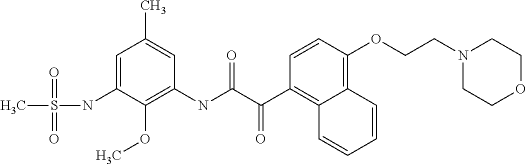 | B |
| 22 | 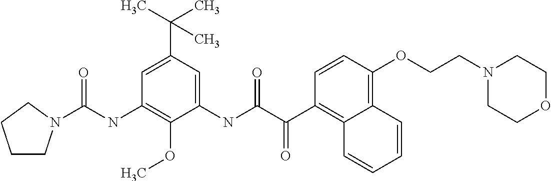 | B |
| 23 | 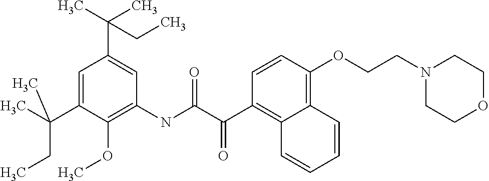 | B |
| 24 | 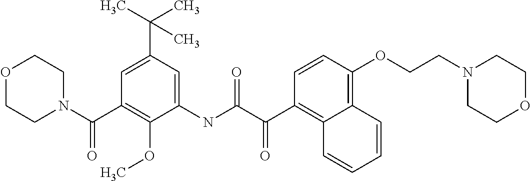 | B |
| 25 | 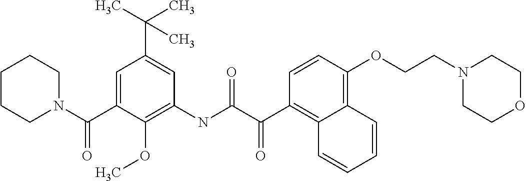 | B |
| 26 | 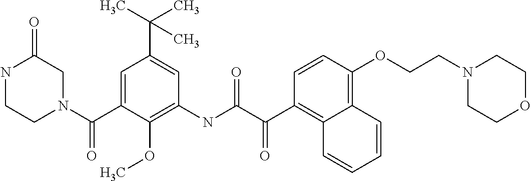 | B |
| 27 | 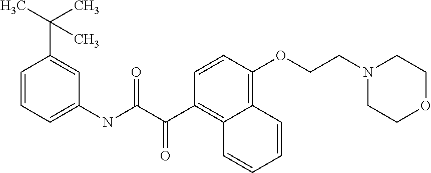 | C |
| 28 | 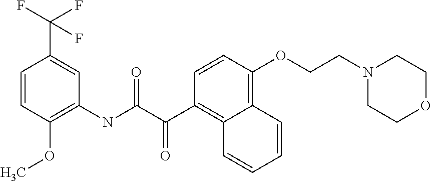 | C |
| 29 | 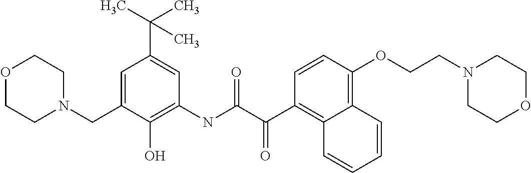 | C |
| 30 | 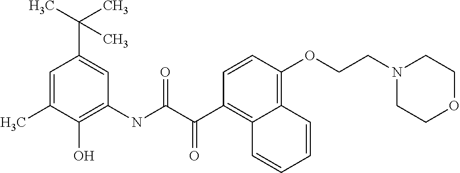 | C |
| 31 | 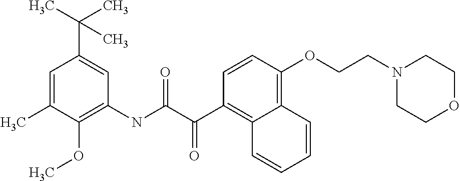 | C |
| 32 | 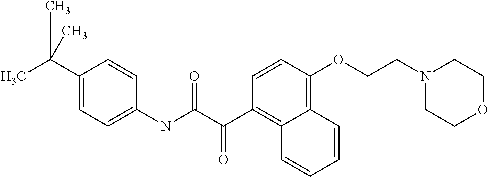 | C |
| 33 | 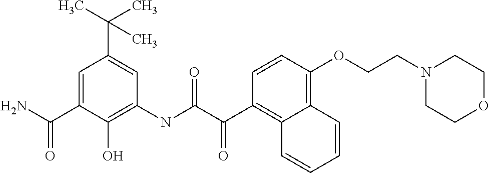 | C |
| 34 | 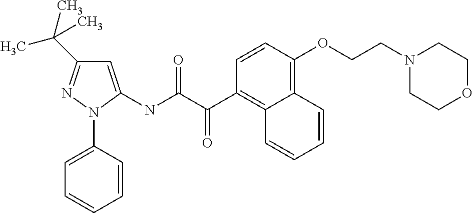 | C |
| 35 | 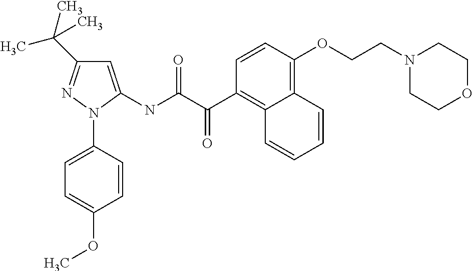 | C |
| 36 | 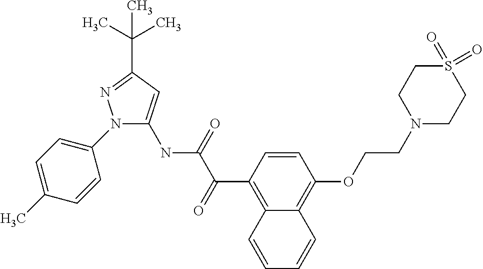 | C |
| 37 | 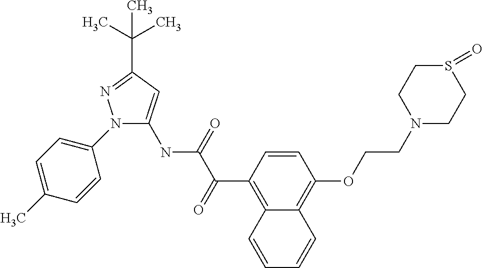 | C |
| 38 | 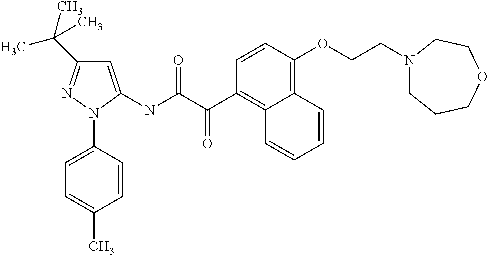 | C |
| 39 | 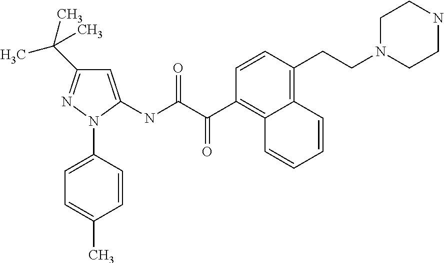 | C |
| 40 | 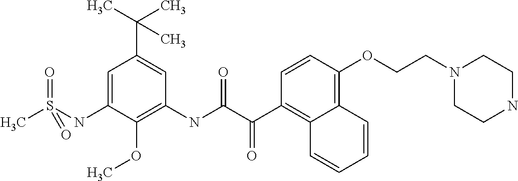 | C |
| 41 | 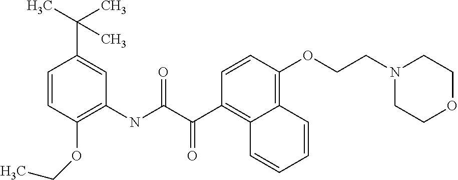 | C |
| 42 | 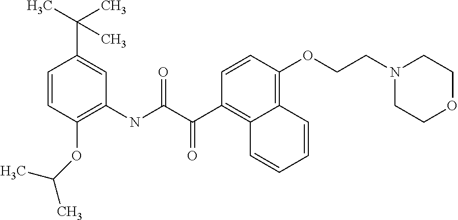 | C |
| 43 | 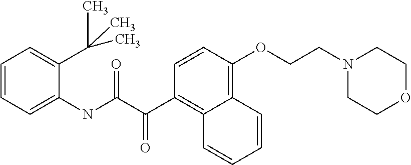 | C |
| 44 | 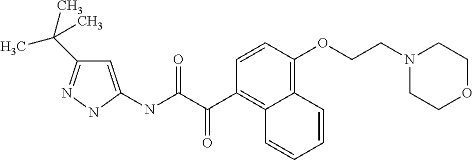 | C |
| 45 | 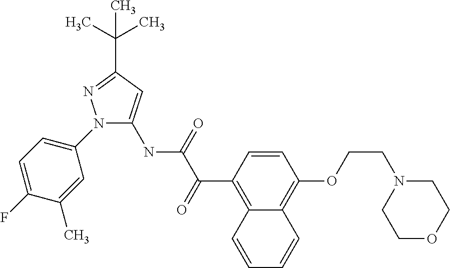 | C |
| 46 | 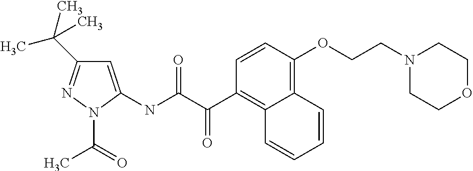 | C |
| 47 | 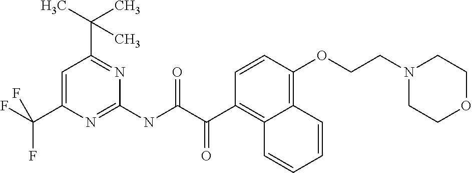 | C |
| 48 | 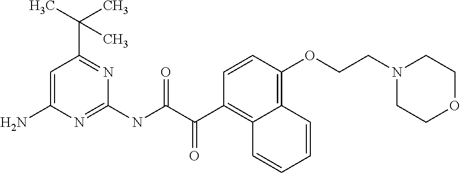 | C |
| 49 | 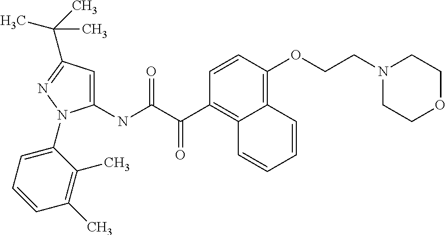 | C |
| 50 | 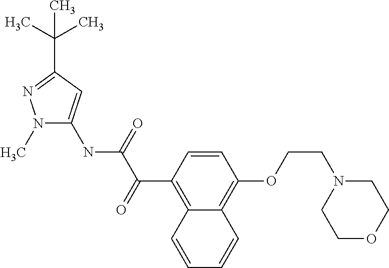 | C |
| 51 | 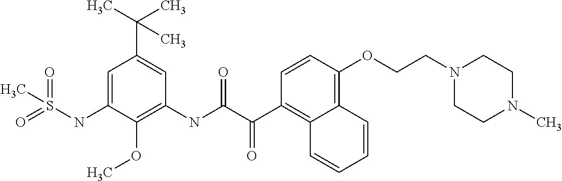 | C |
| 52 | 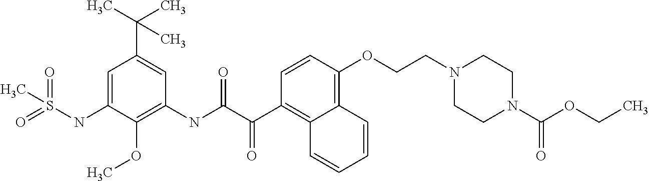 | B |
| 53 | 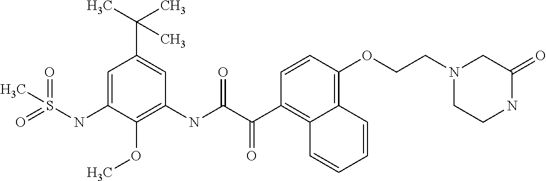 | C |
| 54 | 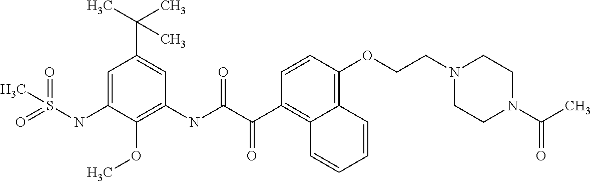 | B |
| 55 | 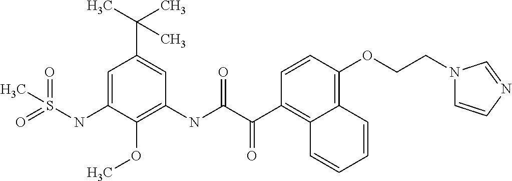 | B |
| 56 | 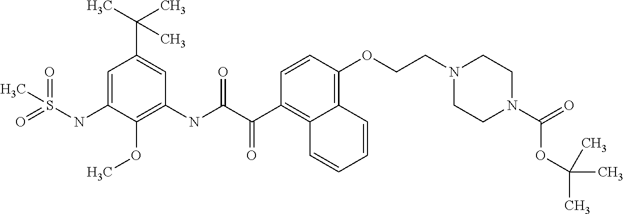 | B |
| 57 | 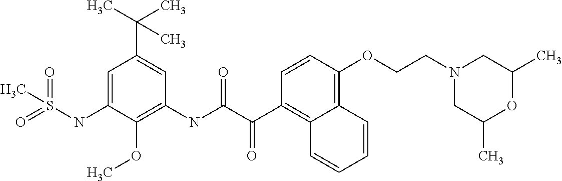 | B |
| 58 | 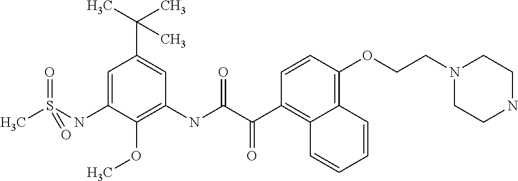 | C |
| 59 | 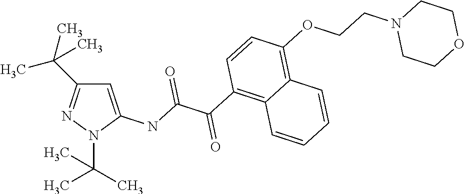 | C |
| 60 | 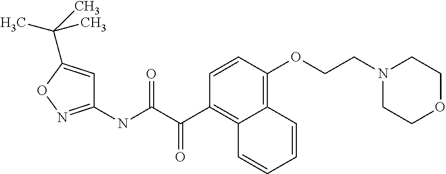 | C |
| 61 | 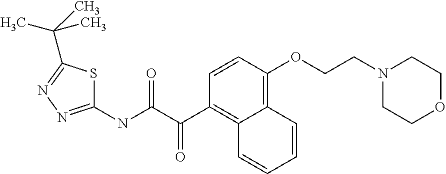 | C |
| 62 | 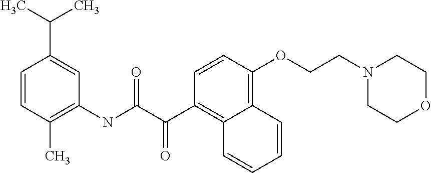 | B |
| 63 | 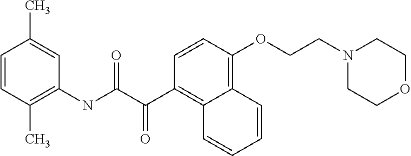 | C |
| 64 | 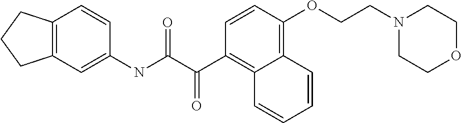 | C |
| 65 | 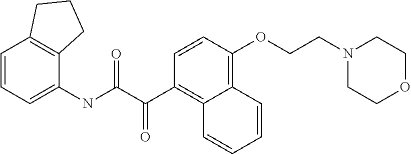 | B |
| 66 | 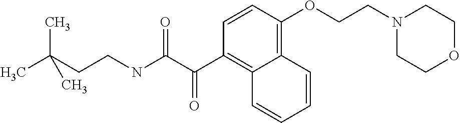 | C |
| 67 | 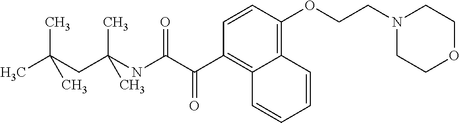 | C |
| 68 | 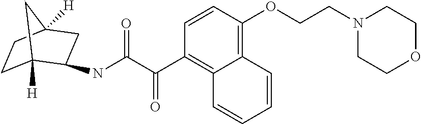 | C |
| 69 | 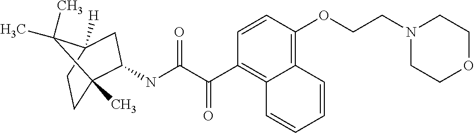 | C |
| 70 | 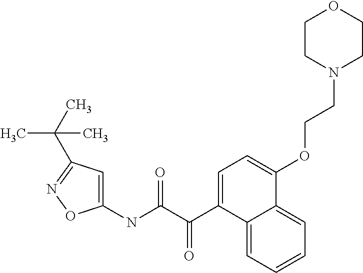 | C |
| 71 | 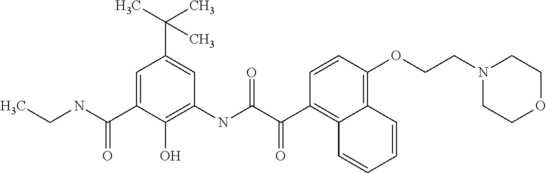 | B |
| 72 | 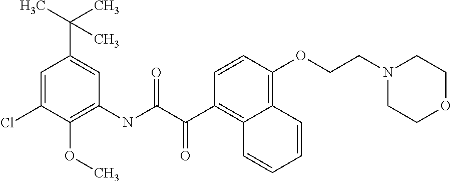 | B |
| 73 | 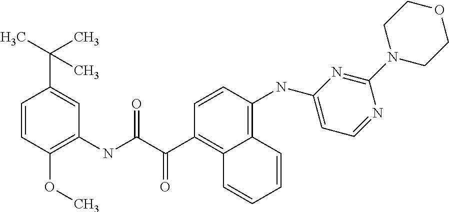 | C |
| 74 | 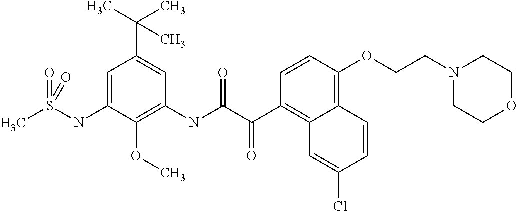 | B |
| 75 | 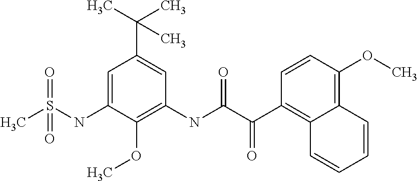 | B |
| 76 | 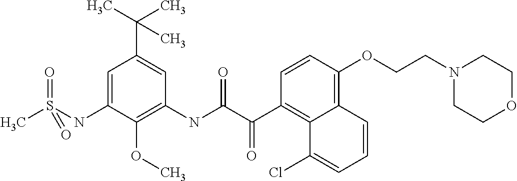 | C |
| 77 | 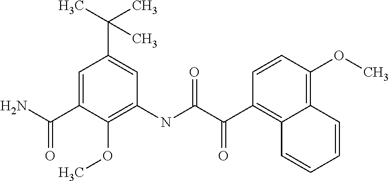 | C |
| 78 | 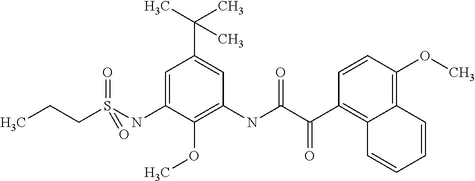 | B |
| 79 | 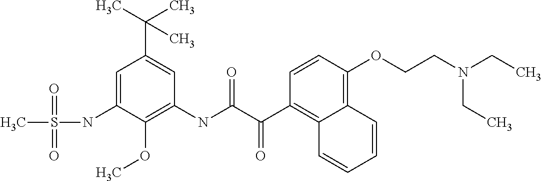 | C |
| 80 | 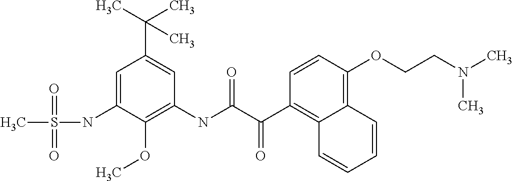 | C |
| 81 | 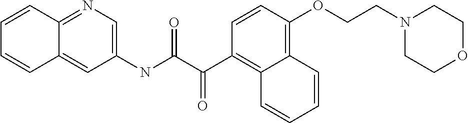 | C |
| 82 | 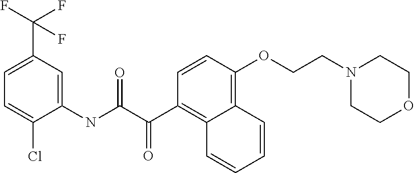 | C |
| 83 | 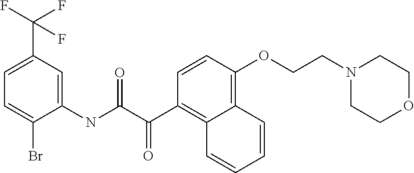 | C |
| 84 | 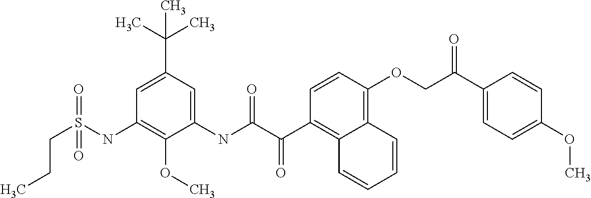 | C |
| 85 | 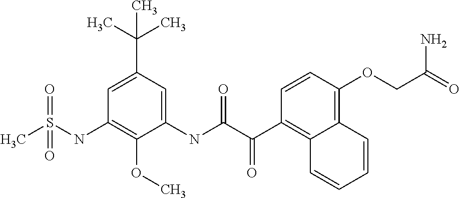 | C |
| 86 | 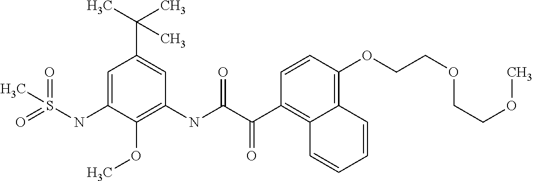 | C |
| 87 | 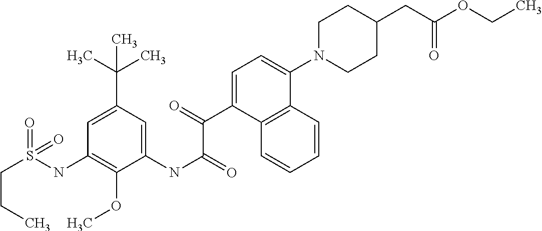 | C |
| 88 | 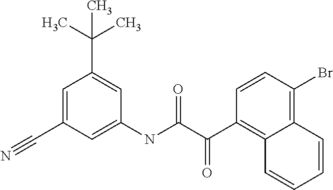 | C |
| 89 | 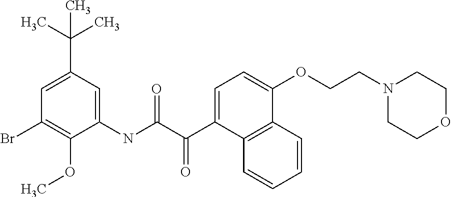 | B |
| 90 | 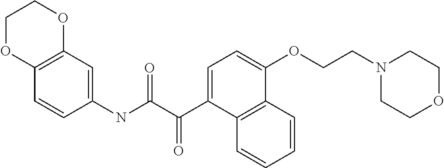 | C |
| 91 | 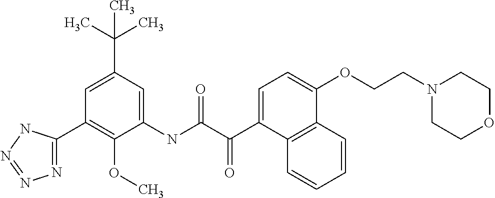 | C |
| 92 | 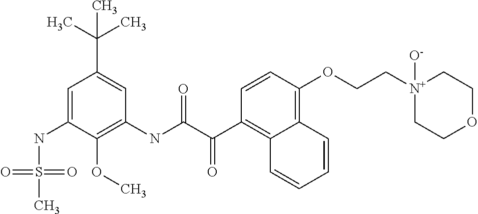 | B |
| 93 | 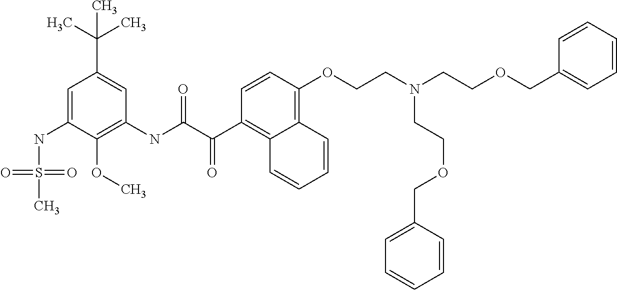 | C |
| 94 | 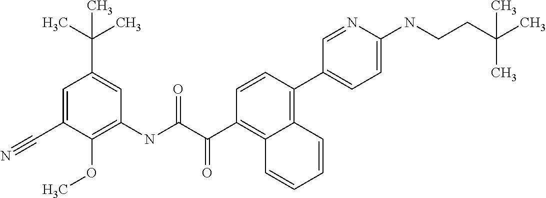 | C |
| 95 | 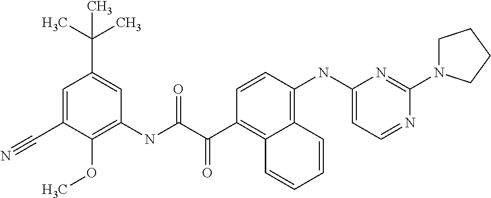 | C |
| 96 | 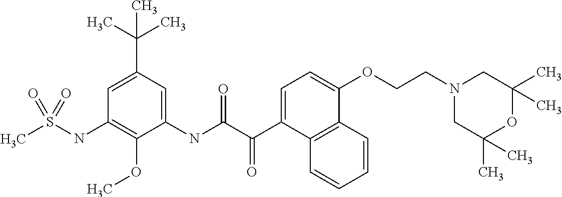 | B |
| 97 | 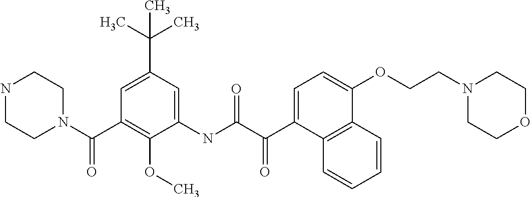 | C |
| 98 | 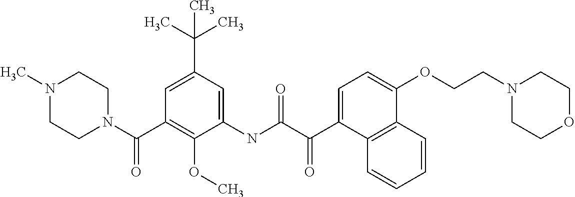 | C |
| 99 | 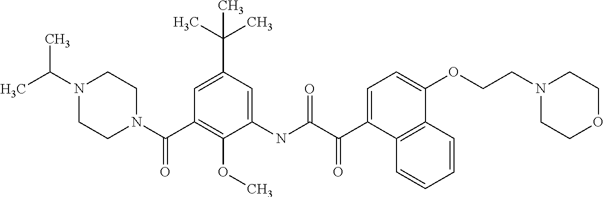 | B |
| 100 | 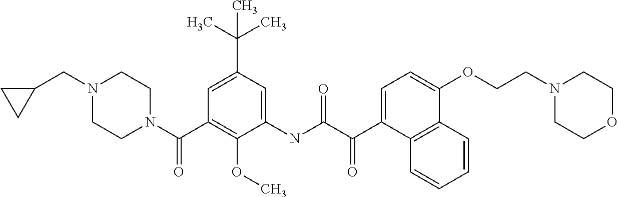 | C |
| 101 | 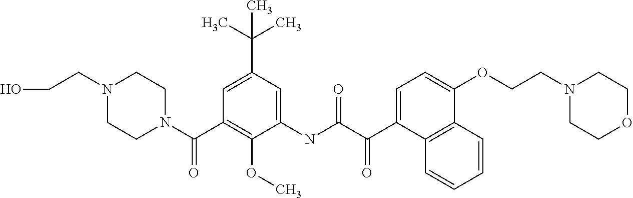 | C |
| 102 | 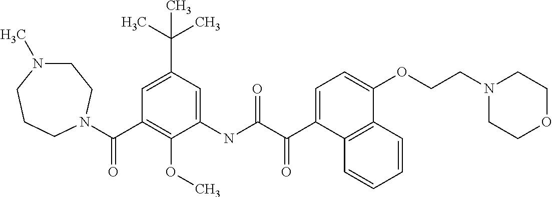 | C |
| 103 | 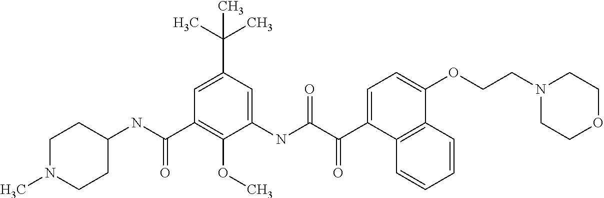 | C |
| 104 | 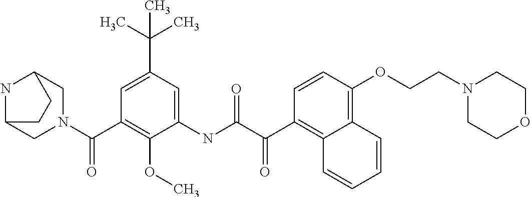 | C |
| 105 | 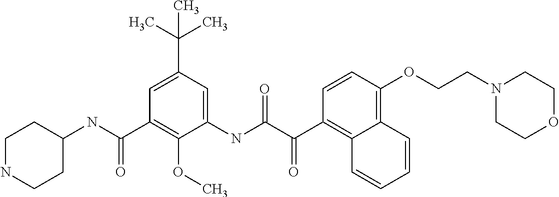 | C |
| 106 | 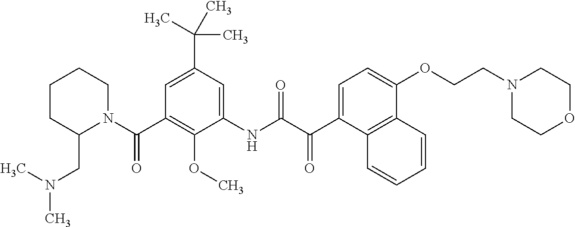 | C |
| 107 | 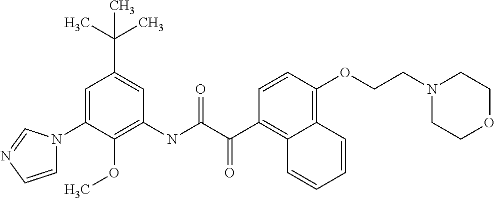 | B |
| 108 | 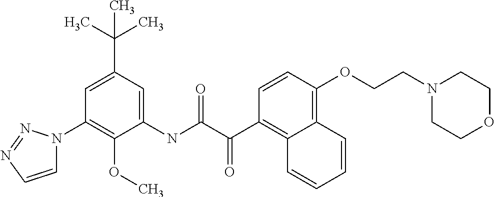 | B |
| 109 | 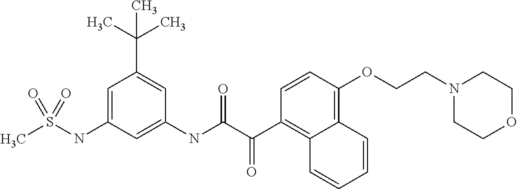 | B |
| 110 | 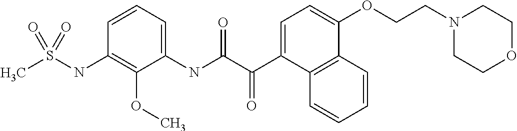 | C |
| 111 | 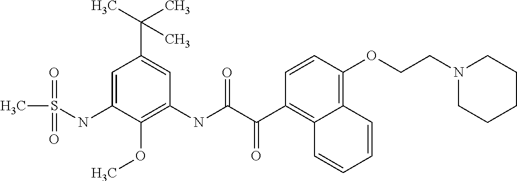 | B |
| 112 | 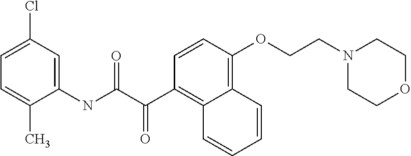 | C |
| 113 | 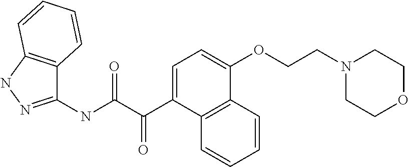 | C |
| 114 | 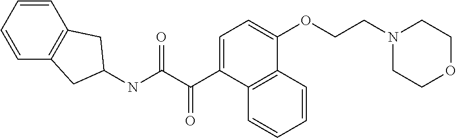 | C |
| 115 | 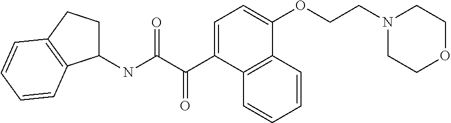 | C |
| 116 | 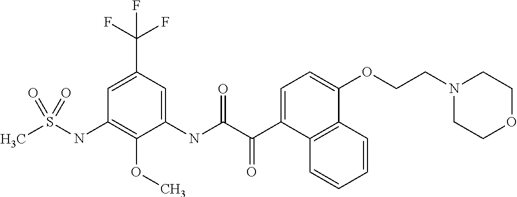 | C |
| 117 | 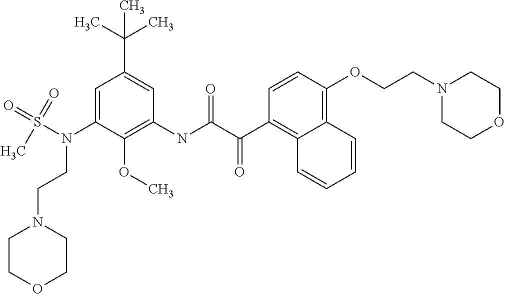 | C |
| 118 | 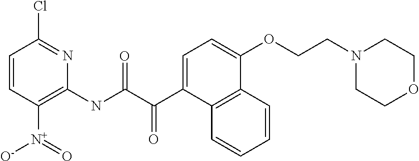 | C |
| 119 | 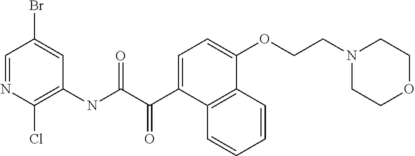 | B |
| 120 | 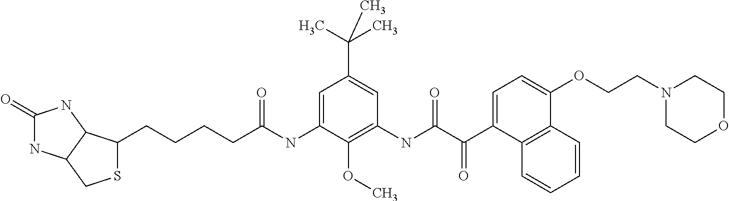 | B |
| 121 | 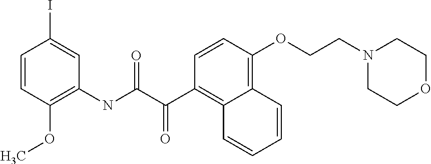 | B |
| 122 | 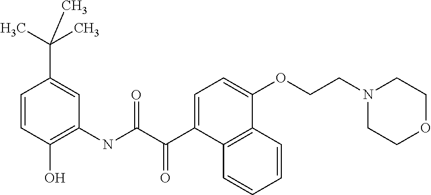 | B |
| 123 | 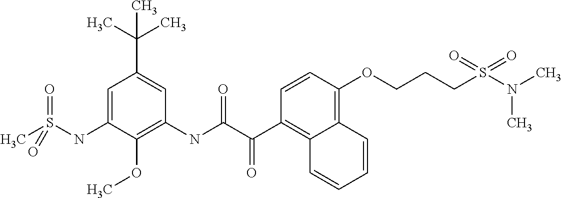 | B |
| 124 | 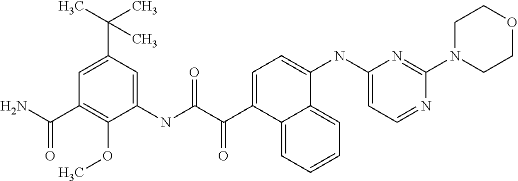 | C |
| 125 | 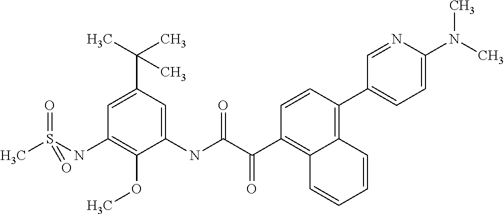 | C |
| 126 | 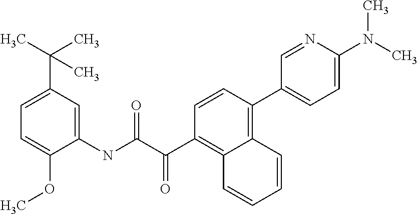 | C |
| 127 | 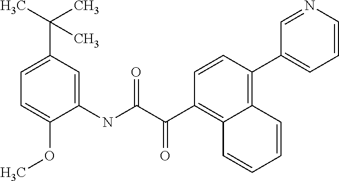 | C |
| 128 | 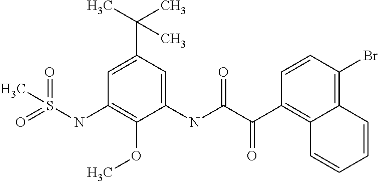 | C |
| 129 | 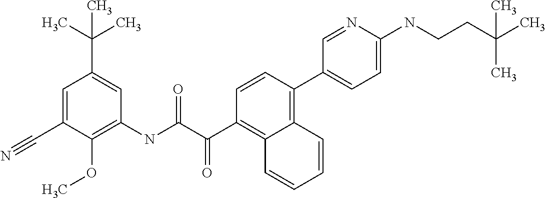 | C |
| 130 | 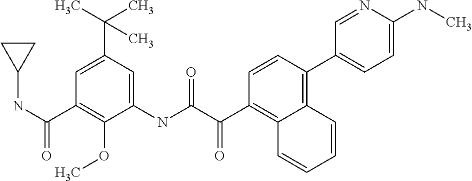 | C |
| 131 | 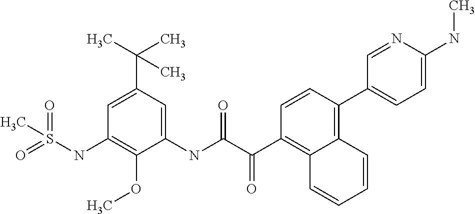 | C |
| 132 | 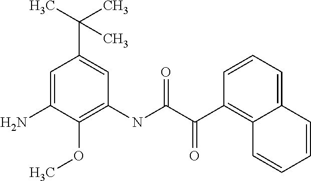 | C |
| 133 | 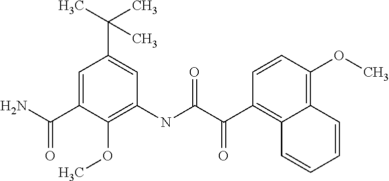 | C |
| 134 | 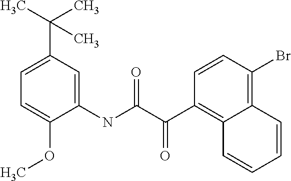 | B |
| 135 | 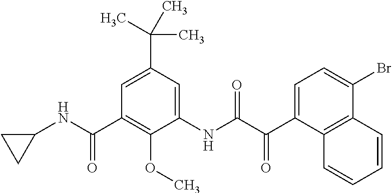 | A |
| 136 | 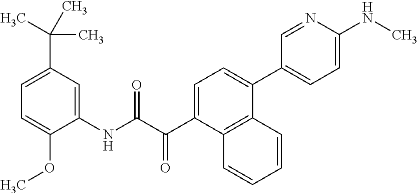 | A |
| 137 | 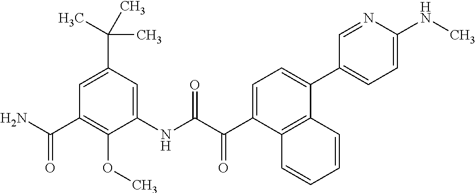 | A |
| 138 | 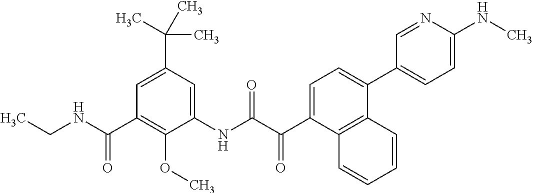 | A |

### Example 4

### HCV Replicon Assay

HCV1a replicon (Huh7 cells stably replicating HCV genotype 1a subgenomic replicon) were treated with serial dilutions of the test compound for 72 hrs. Replication efficiency was monitored by HCV genomic RNA amplification using Taqman analysis.

Alternatively, Hut1b cells (Huh7 cells stably replicating HCV genotype 1b subgenomic replicon) were treated with serial dilutions of the test compound for 72 hours. Replication efficiency was monitored by ELISA assay using anti-HCV NS5A monoclonal antibody (Virogen).

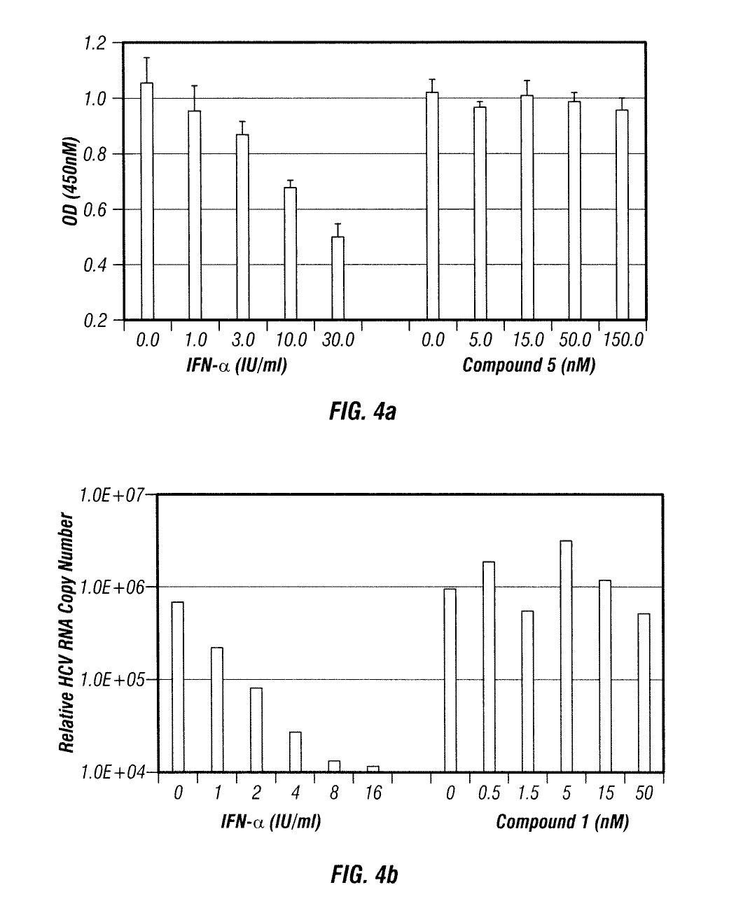

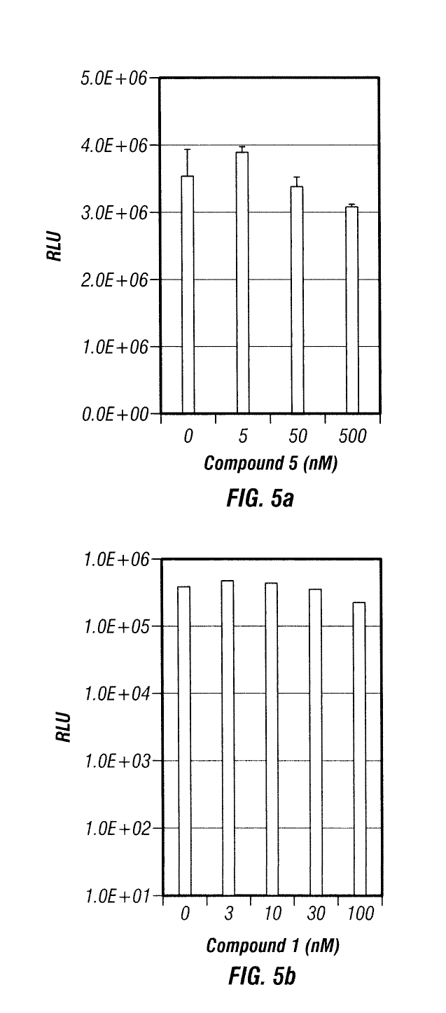

Results: To confirm the mode of action of Compounds 1 and 5, replicon assays were used as counter screens to rule out inhibition of intracellular virus replication. Increasing amounts of compound were inoculated onto HCV1b replicon cells and incubated for 3 days. Following incubation, HCV replication was monitored with NS5A protein expression by ELISA. IFN-α was treated as a positive control for HCV replication inhibition and showed a dose dependent inhibition of HCV replication, while Compounds 1 and 5 each showed no measurable effect, indicating that anti-HCV activity is likely not related to either genome amplification or polyprotein processing (FIG. 4). In addition, no cellular toxicity on Huh7 cells was observed with up to 100 μM of Compound 1 or 500 nM of Compound 5 (FIG. 5). These results are consistent with Compound 5 as an entry inhibitor in the pseudovirus assay (data not shown).

### Example 5

### BVDV Plaque-Forming Assays

Bovine viral diarrhea virus (BVDV) is a closely related virus to HCV, belonging to the same Family, Flaviviridae. To validate the specificity of antiviral activity, BVDV infection was done with MDBK cells in the presence of compounds provided herein. MDBK cells (70 to 80% confluent) were infected with a diluted virus. Following 1 h of adsorption at 37° C., cells were washed once with DMEM, overlaid with 1.5% low-melting-point (LMP) agarose (Gibco-BRL) in MEM containing 5% HS, and incubated at 37° C. To assay for plaque-forming virus, after 3 days monolayers were fixed with 3.7% formaldehyde for 2 h at room temperature, the agarose plugs were removed, and the monolayers were stained with crystal violet.

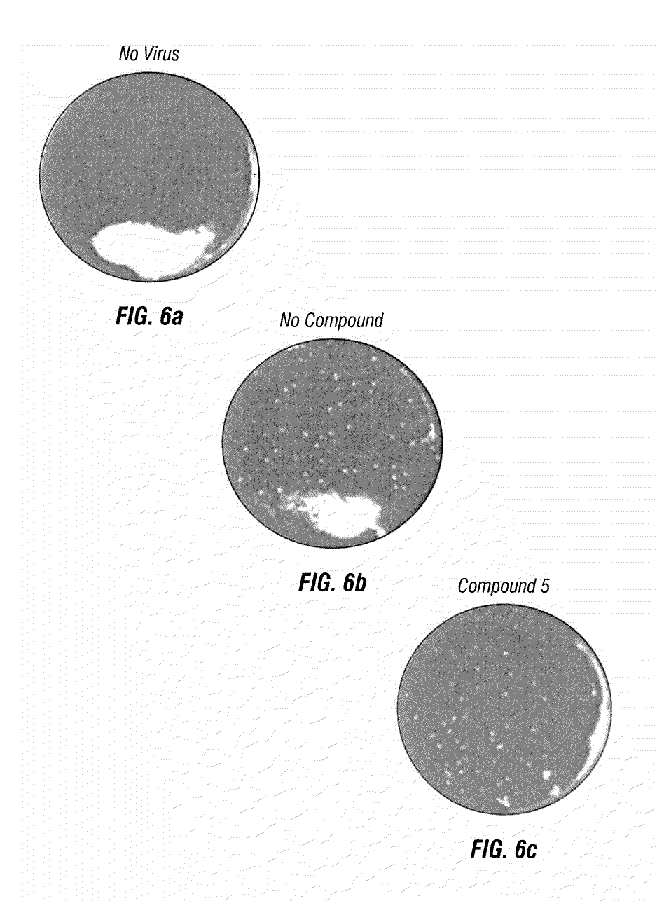

Results: No significant inhibitory effect on BVDV infection was observed even at 1 μM for Compound 5 (FIG. 6).

IF analysis: Following aspiration of supernatant from a 24-well plate, 1 ml of ice cold methanol was added to each well and the plate was stored overnight at −20° C. The following day, methanol was removed and the plates were air-dried at room temperature for about 30 minutes. The plate was then rinsed/rehydrated with two washes of PBS (˜5 minutes/wash). To block non-specific antibody binding, cells were incubated in blocking buffer (1% BSA, 0.2% skim milk in PBS) for 1 hour at room temperature. Primary antibody (mouse anti-HCV Core; Affinity BioReagents, MA1-080) was diluted 1:1000 in PBS+0.1% Tween-20 and incubated overnight at 4° C. on rocker (˜200 μl of antibody/well). The next day, wells were washed with PBS (three washes of 5 minutes each) and then incubated for 30 minutes at room temperature with a secondary antibody: goat anti-mouse_AlexaFluor488 (1:1000 dilution in PBS/Tween; Molecular Probes/Invitrogen #A11001). The cells were then washed with PBS three times (5 minutes each), including once with Hoechst dye (0.4 ug/ml; Molecular Probes/Invitrogen #H3570) to counterstain the nuclei of cells. Immunofluorescence staining was analyzed using 10× objective on a Zeiss fluorescent microscope.

### Example 6

### Inhibition of Viral Entry of Both HCV1a and HCV 2a

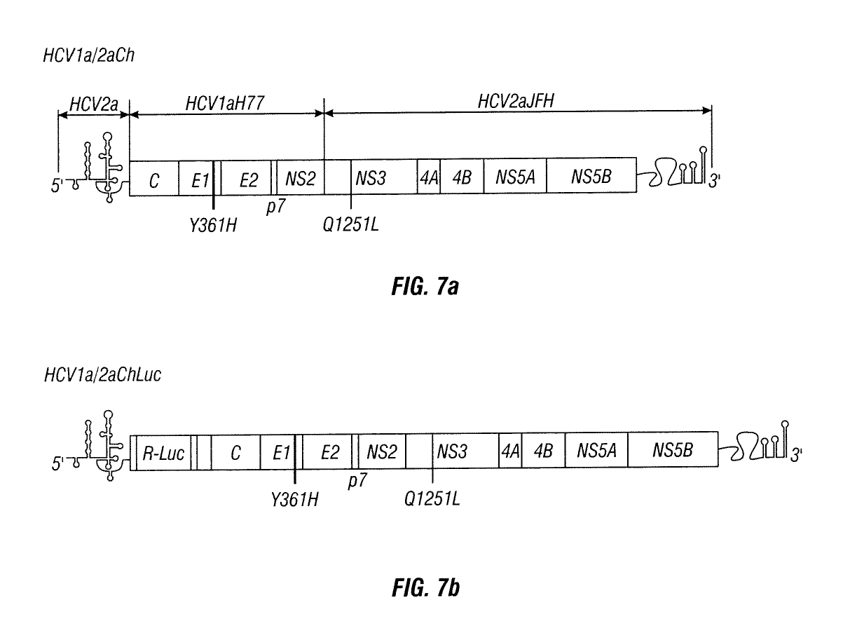

Since HCV genotype 1 is the most prevalent genotype among the variants of HCV, we constructed an HCV1a/2a chimeric infectious clone with two cell-culture adaptive mutations introduced. Sequences for the structural proteins (including C, E1, and E2), p7, and NS2 were derived from HCV genotype 1a H77 strain. The rest of nonstructural proteins (NS3 to NS5B) and 3′ NTR were derived from HCV genotype 2a JFH strain. 5′NTR was originated from HCV2aJ6 (FIG. 7). Yi and her colleagues reported that two cell-culture adaptive mutations, Y361H and Q1251L, significantly enhanced the infectivity of HCV1a/2a chimeric virus in cell culture (J. Virol. 2007 81: 629-63 8). Therefore, both HCV1a/2aCh virus and HCV1a/2aChLuc virus with Y361H and Q1251L mutations were successively produced in Huh7 cells and used for HCV entry assay.

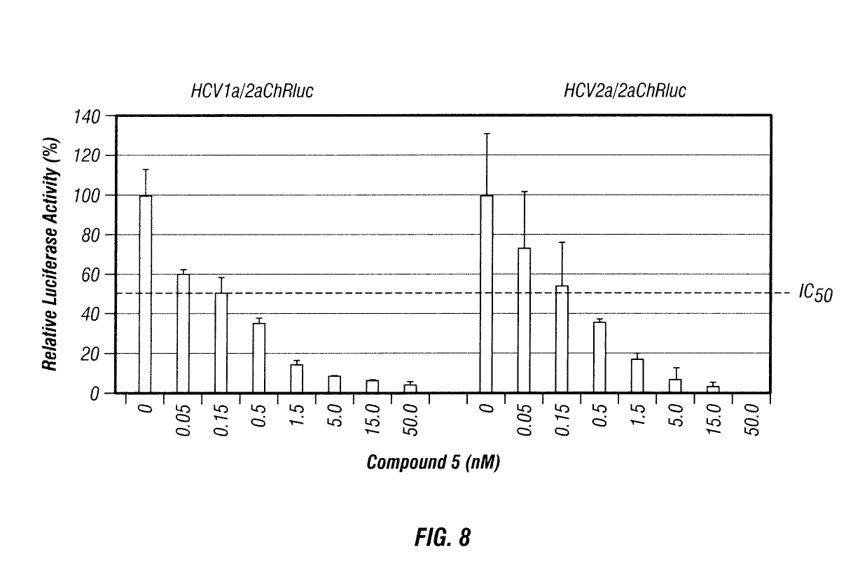

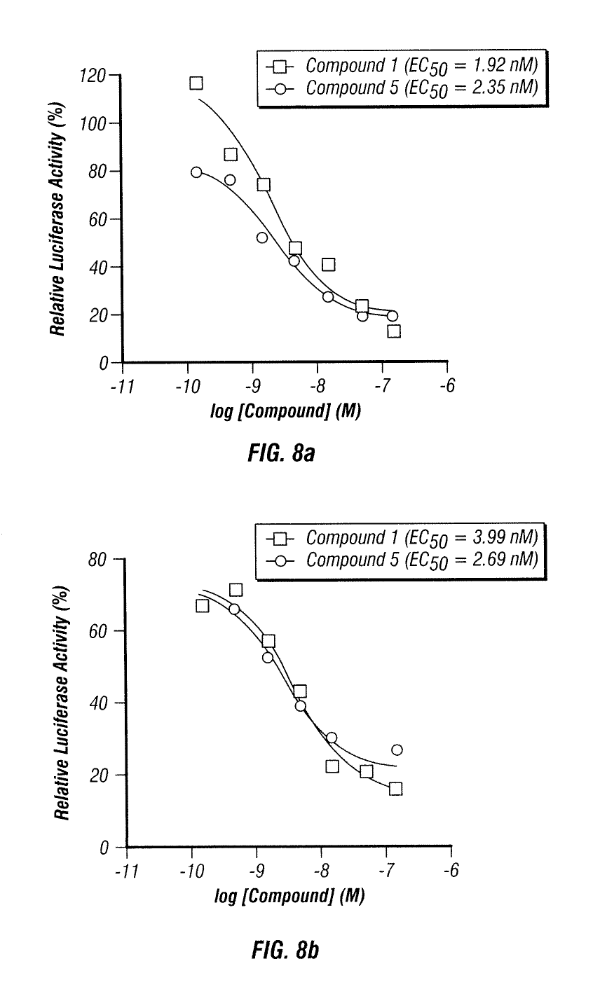

Results: the HCV1a chimeric virus was inhibited by both Compound 1 and Compound 5 to a very similar extent as observed with the HCV2a chimeric virus (FIG. 8).

### Example 7

### Effects of the Combination a Viral Entry Inhibitor with IFN-α

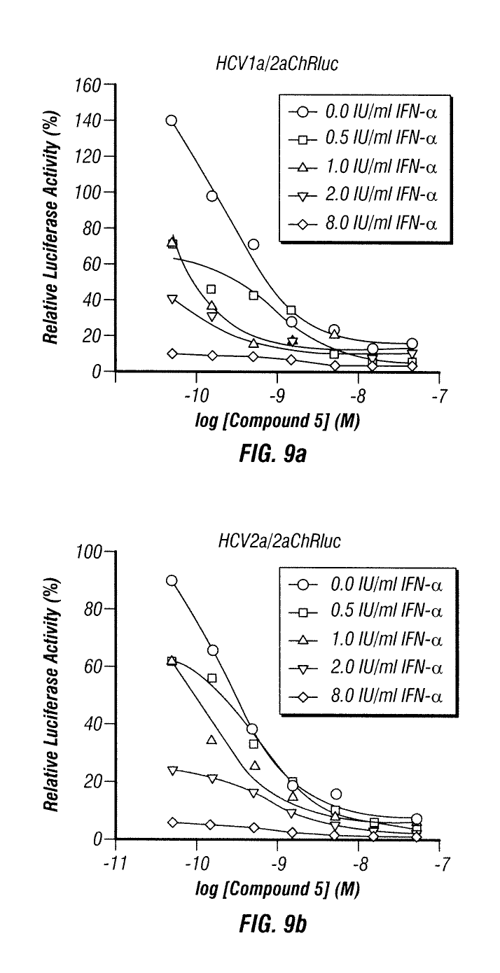

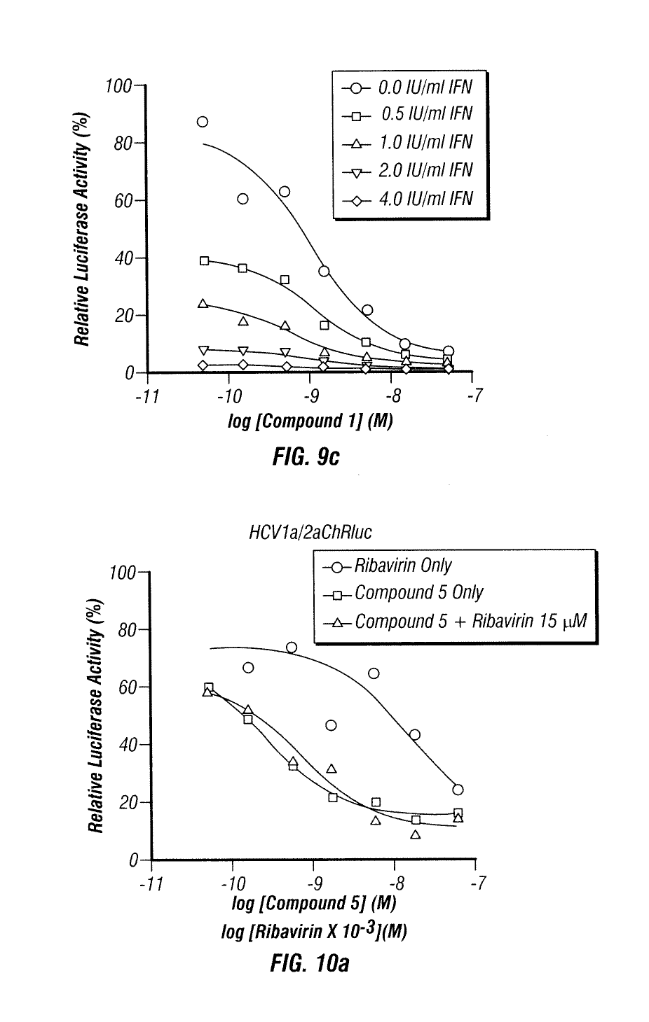

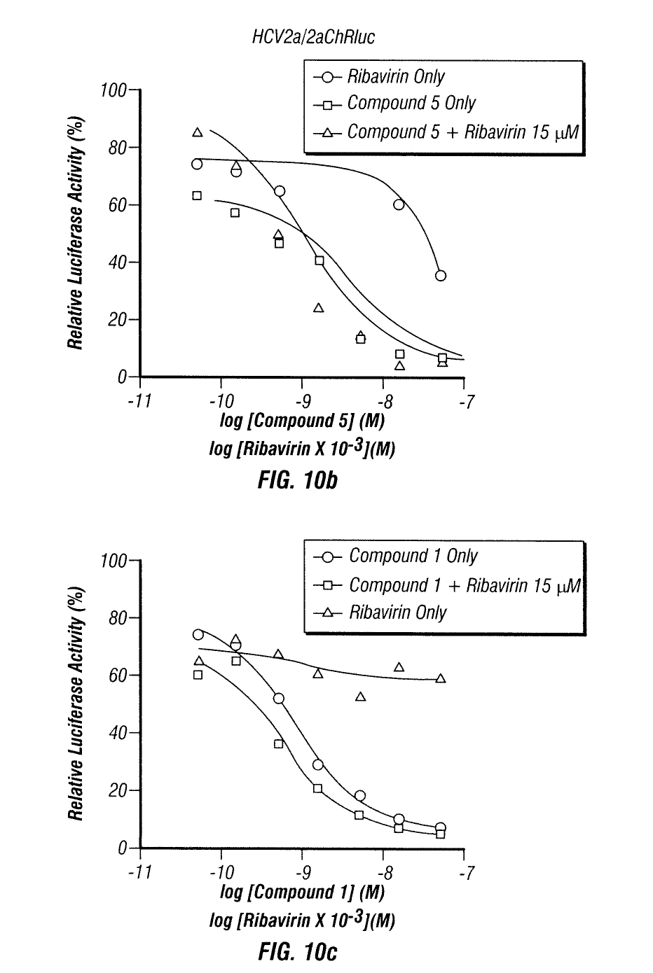

The current standard of care for the treatment of chronic hepatitis C is the combination of pegylated IFN-α (interferon-alpha) and ribavirin. Since IFN-α, but not ribavirin, exhibits strong antiviral activities in vitro and in patients, we evaluated the effect of the combination of either Compound 1 or Compound 5 and IFN-α using HCVcc infection systems in vitro. Huh7 cells were incubated with various concentrations of Compound 1 or interferon alone, or combinations of both for 72 hrs, and the anti-HCV activity was measured by luciferase assays. The same was experiment was performed for Compound 5. As shown in FIG. 9, the combination of either Compound 1 or Compound 5 with IFN-α always resulted in a reduction in the level of luciferase activity greater than that achieved by each agent alone. These results indicate that the combination of Compound 1 and IFN-α and Compound 5 and IFN-α each has an additive to moderate synergistic effect on inhibition of HCV infection in cell culture.

### Example 8

### Effects of the Combination a Viral Entry Inhibitor with Ribavirin

It is known that ribavirin alone does not exhibit strong antiviral activities in vitro, and is a key component of the standard of care in patients. We investigated whether Compound 1 and Compound 5 had antagonistic activities in combination with ribavirin. Varying amounts of each compound were coincubated with a high concentration of ribavirin (15 μM). Data indicate that ribavirin alone did not have significant inhibitory effect on the infectivity of HCV1a chimeric virus (a) and 2a chimeric virus (b). However, no antagonistic effect was discerned in the combinations of Compound 1 and ribavirin, and Compound 5 and ribavirin.

### Example 9

### Effects of the Combination of a Viral Entry Inhibitor with a HCV Protease Inhibitor (VX-950)

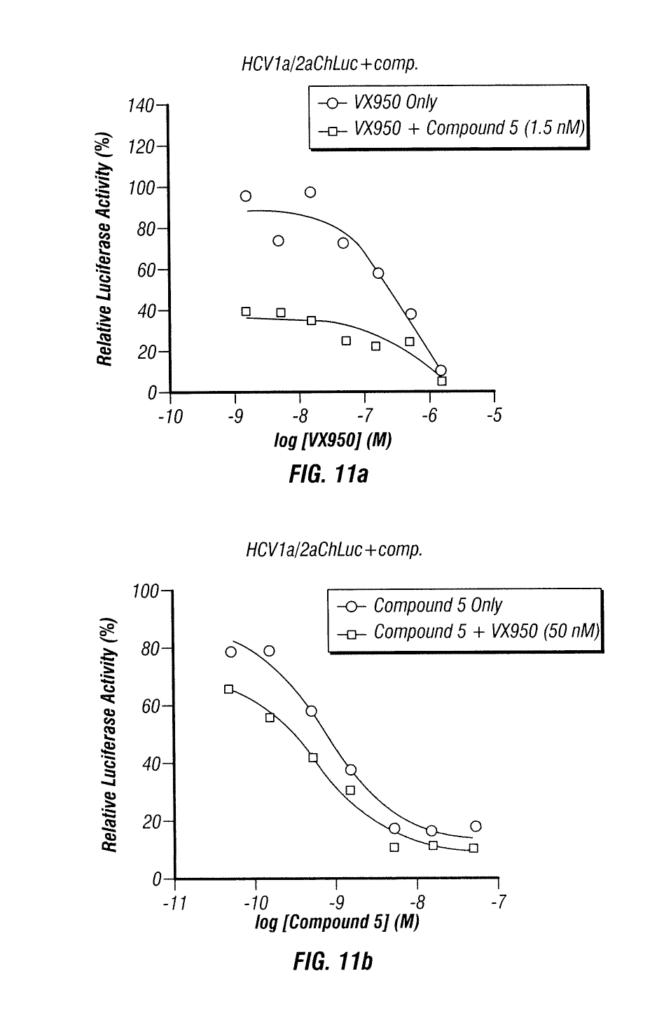

Additional combination studies of Compound 1 and Compound 5 with the HCV NS3 protease inhibitor VX-950 were conducted. Compound 1 and Compound 5 were tested singly and in combination with VX-950. The ratio of the two compounds, based on the compound's EC₅₀, remained fixed across the dosing range. The potential cytotoxicity of individual compounds was assessed with a luminescent ATP-based cell viability assay (Cell Titer-Glo; Promega). Cell culture and luciferase compound assays were performed as previously described in Example 3. The results demonstrated additive to slightly synergistic effects of these compounds (FIG. 11).

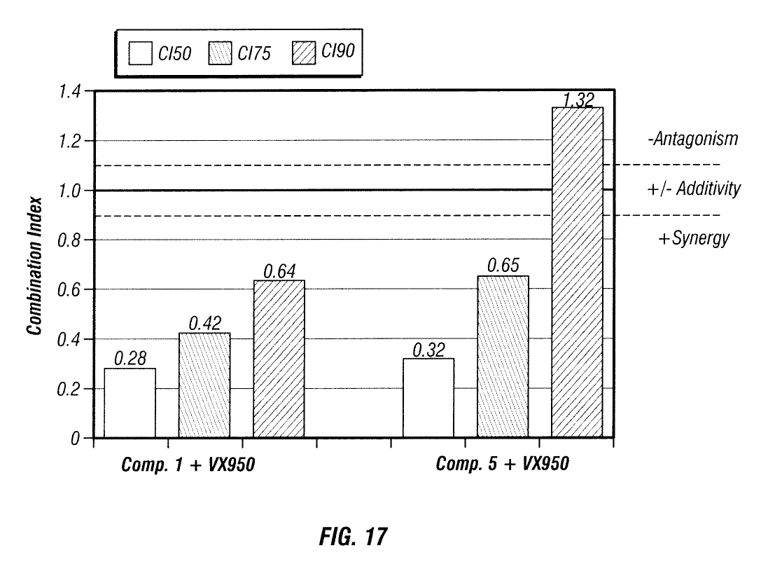

Compound interactions were quantified by the approach described by Chou and Talalay, relying on the median effect principle and the multiple-drug effect equation. See Chou, T. C., and P. Talalay, Adv. Enzyme Regul., 1984, 22: 27-55. Isobolograms were generated for each combination tested and were used to determine the additivity, synergism or antagonism of inhibitor combinations. Combination indices (CI) were determined with Calcusyn (Biosoft) for each experiment at the EC50, EC75, and EC90 of the combination. By convention, a CI of <0.9 was considered synergistic, a CI of ≧0.9 or ≦1.1 was considered additive, and a CI of >1.1 was deemed antagonistic. As shown in FIG. 17, numerical values above the bars are mean CI. The dotted lines at 0.9 and 1.1 represent the bounds of an additive interaction: +, synergy; ±, additivity; −, antagonism. Combination indices (CI) were determined with Calcusyn (Biosoft) for each experiment at the EC₅₀, EC₇₅, and EC₉₀ of the combination. By convention, a CI of <0.9 was considered synergistic, a CI of ≧0.9 or ≦1.1 was considered additive, and a CI of >1.1 was deemed antagonistic.

### Example 10

### A Protease Mutant (NS3:A156S) Showed High Resistance Level to VX-950, but not to Compounds 1 and 5

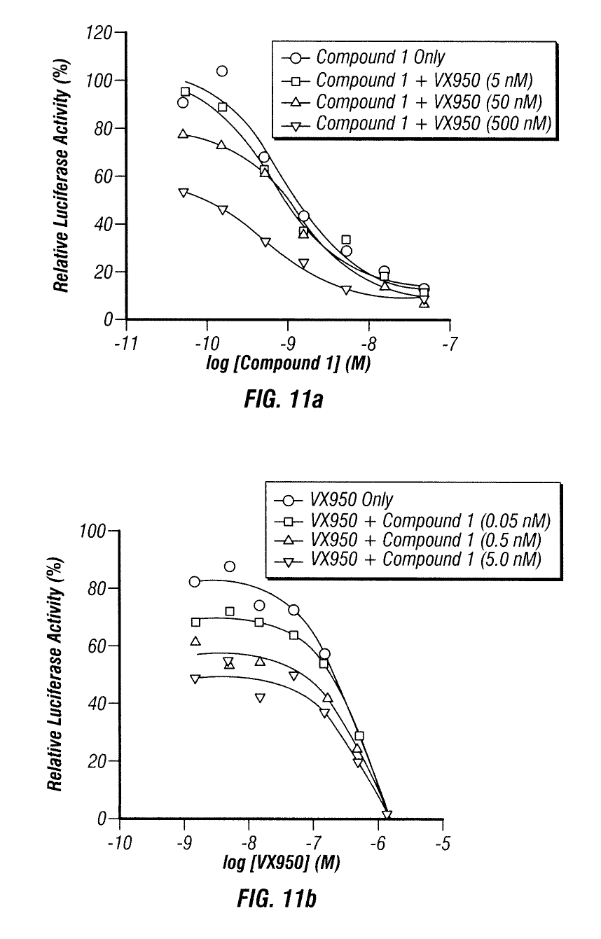

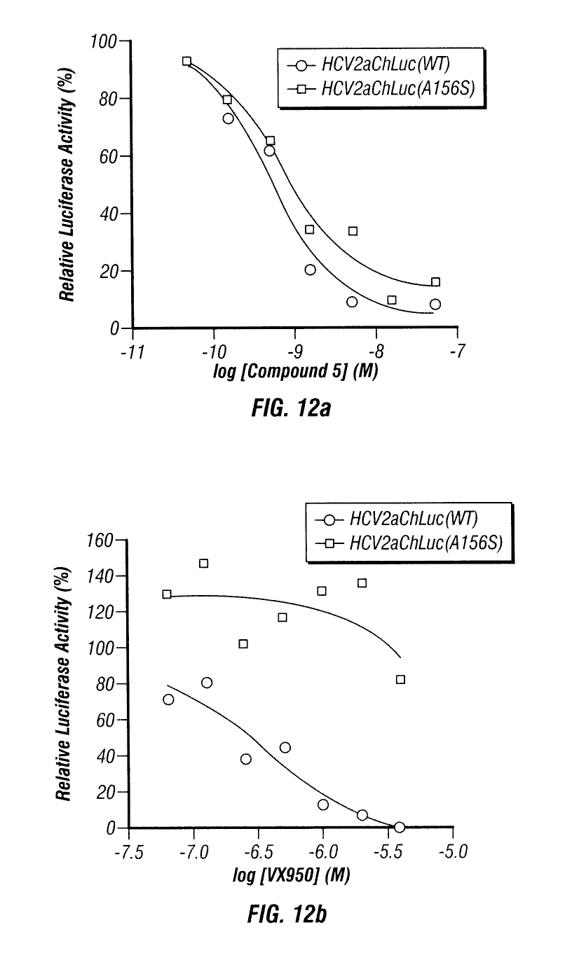

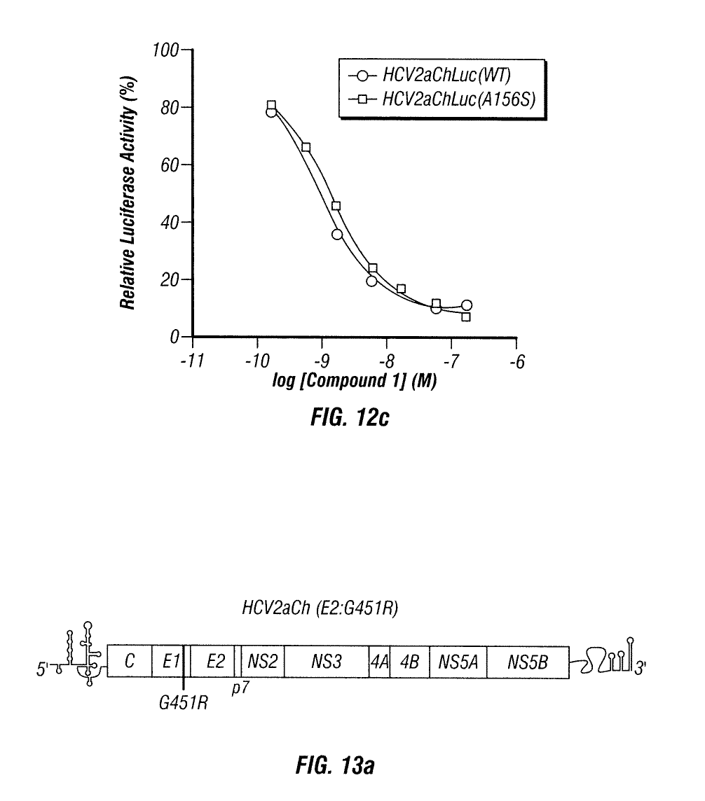

The A156T mutation in the NS3 coding region has been shown to confer resistance to many HCV protease inhibitors, such as SCH-503034 (boceprevir, Schering-Plough), SCH-6 (Schering-Plough), BILN-2061 (Boehringer Ingelheim), and VX-950 (Vertex). Therefore, we introduced A156S mutation in the backbone of HCV2aChLuc genome and investigated the infectivity of the mutant virus in the presence of compounds. As expected, HCV2aChLuc (NS3:A1 56S) showed high resistance to VX-950 as compared to the wild type of HCV2aChLuc. However, no significant difference was observed in the inhibition of infectivity of both the wild type and the mutant virus by Compound 1 or Compound 5, indicating the lack of cross-resistance between a protease inhibitor and a viral entry inhibitor, as expected (FIG. 12).

### Example 11

### SR-B1 is a Potential Target of Compounds 1 and 5

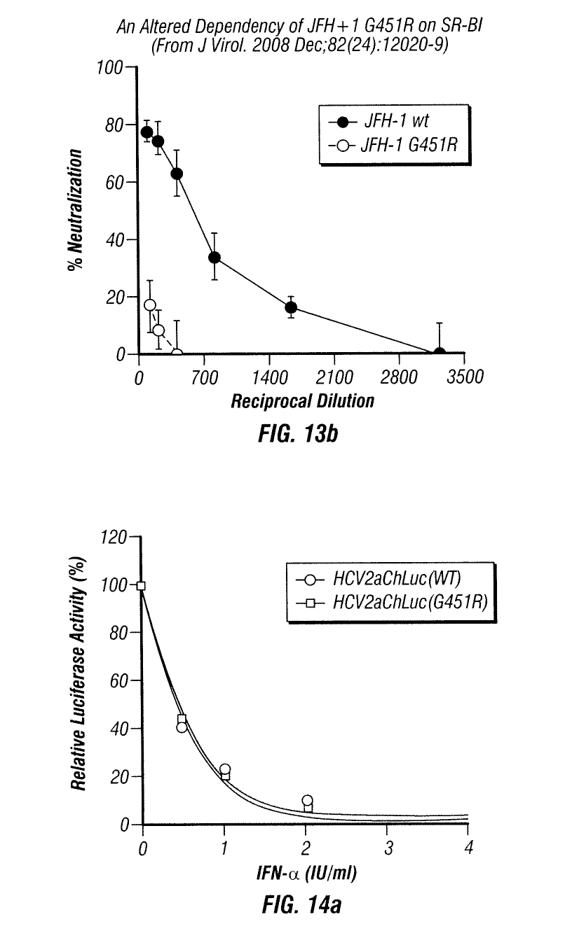

Compound 5 raised HDL levels in mice and in human subjects, but not in mice with a knock-out in the SR-B1 gene (A. Tall, manuscript in preparation), suggesting that it targets the SR-B1 pathway. Since SR-B1 is also known to be one of the essential cellular receptors required for HCV entry, we were interested to know if Compounds 1 and 5 inhibits HCV entry via SR-B1. HCV E2 protein interaction with SR-B1 is believed to occur via hypervariable region 1 located within the N-terminal region of the E2 glycoprotein. Zhong and colleagues reported that a glycine-to-arginine mutation at position 451 (G451R) in E2 promoted JFH-1 infectivity (J. Viol. 2006, 80:11082). Grove and colleagues demonstrated that JFH-1 G451R mutation had a reduced dependency on SR-BI and increased binding to CD81 (J. Viol. 2008, 82:12020) (FIG. 13b). We introduced the E2:G451R mutation into the backbone of HCV2aCh (FIG. 13a) to test whether this mutation may alter the inhibitory effect of Compounds 1 and 5 on HCV entry mediated by SR-B1.

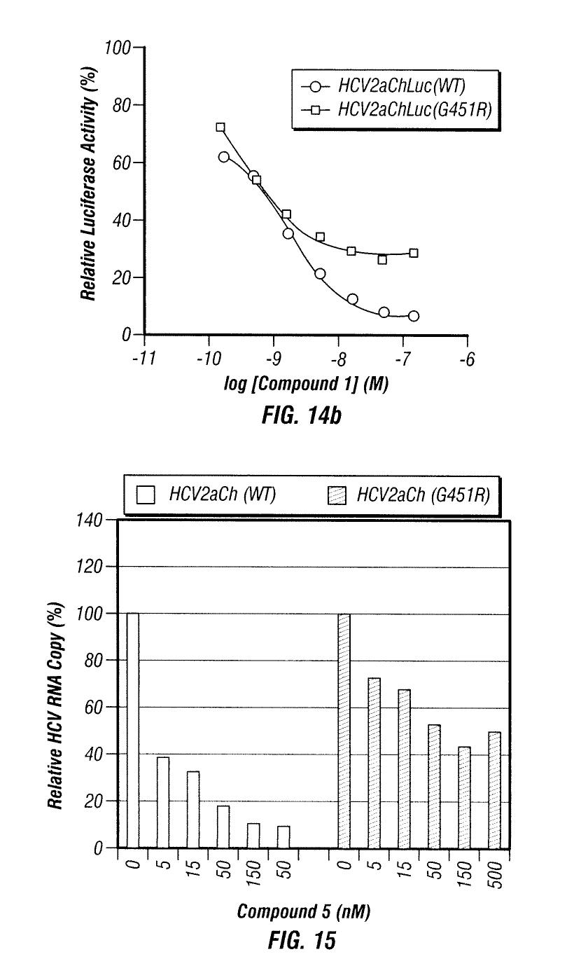

IFN-α was used as a control to show that less sensitivity of the E2 mutant virus to inhibition of Compound 1 compared to that of the parental virus is originated from the viral entry process, not from the replication process. The E2 mutant virus did show greater resistance to Compound 1 relative to the parental type virus, as shown in the luciferase assay for viral infectivity (FIG. 14). However, the responses of the parental and E2 mutant virus to IFN-α showed very identical patterns indicating that anti-HCV activity of Compound 1 is more likely related to viral entry step than to the other steps of HCV life cycle (FIG. 14).

As shown in FIG. 15, the E2 mutant virus did show greater resistance to Compound 5 relative to the wild-type virus, as shown in the Taqman assay for viral RNA copies. Two 24-well plates were seeded with 4×10⁴ cells/well. The following day, cell culture supernatant was aspirated and cells were exposed to HCV2aCh wild type (black bars) and HCV2aCh (E2:G451R) mutant (blue bars), Approximately 250 μl of cell culture virus was added to each well (˜1×10⁴ TCID/well or moi ˜0.25). Immediately after the addition of virus to the well, 2.5 μl of 100× concentration of compound was added to each well—and mixed multiple times with a 100 ul pipet tip—to achieve final compound dilutions: 0 nM, 5 nM, 15 nM, 50 nM, 150 nM, and 500 nM. Compound 5 was tested. Each plate was processed consecutively. After addition of virus and compound the plates were placed in the incubator for ˜3.5 hours (37° C., 5% CO₂). Following incubation, the supernatant was replaced with fresh media (˜1 ml/well) and plates were returned to incubator. Twenty-four hours later (˜27.5 hours following virus exposure to the cells), cells were washed once with 1 ml of PBS and then the cells were processed for Taqman analysis.

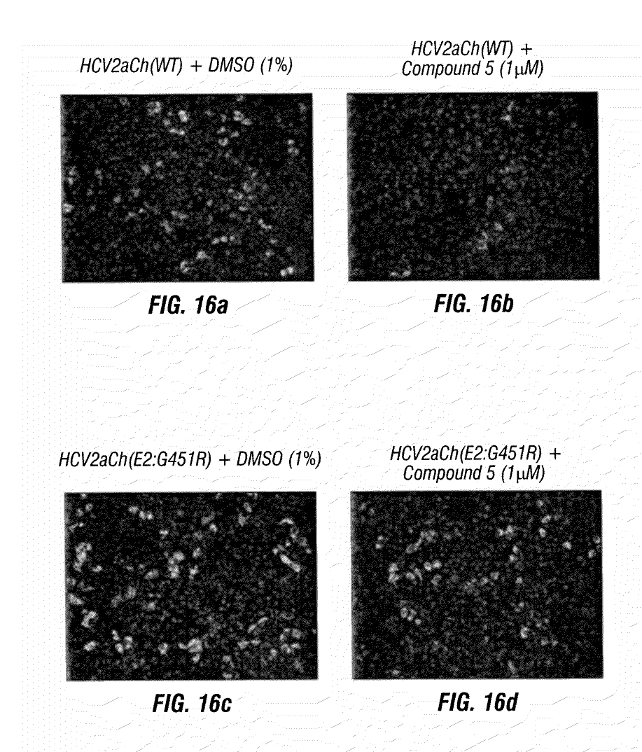

The E2 mutant was also demonstrated to be more resistant to Compound 5 as compared to wild-type virus using immuno-fluorescence as a read-out (FIG. 16).

### Example 12

### Combination Treatment of Compound 5 and Anti-HCV Compounds

Combinations of compound 5 and the following anti-HCV compounds were evaluated in a HCV genotype-2a infectious virus system: interferon-alpha, ribavirin, BILN2061, VX950, VX1 and 2′-C-methyladenosine.

Cell culture. Human hepatoma Huh-7.5.1 cells or Huh7 cells were grown at 37° C. and 5% CO₂ in Dulbecco's modification of Eagle's medium supplemented with 2 mM L-glutamine, 1 mM Sodium pyruvate, 1× Non-essential amino acids mix, 100 units/ml penicillin, 100 μg/ml streptomycin, and 10% fetal bovine serum.

Virus plasmids. The chimeric full-length construct pFL-JC1, a GT2a/2a chimera consisting of structural genes from the J6CF isolate and non-structural genes from the JFH1 isolate, has been described previously. See Backes et al., J. Virol. 2010, 84:5775-89; Schaller et al., J. Virol., 2007, 81:4591-603; Koutsoudakis et al., J. Virol. 2006, 80:5308-20; Pietschmann et al., Proc. Natl. Acad. Sci., 2006, 103:7408-13. The firefly luciferase gene has been inserted to pFL-Jc1 to develop a plamid pFL-Jc1-luc for use as a reporter of viral replication. See Amako et al., J. Virol. 2009, 83:9237-46. Specifically, the pFL-JC1-luc construct can be utilized to develop an infectious HCV cell culture system to test novel HCV inhibitors, including entry inhibitors, in vitro using luciferase expression.

Preparation of infectious HCV. Jc1-luc or HCV2a/2aChRluc RNA was in vitro transcribed using T7 RiboMAX™ Express Large Scale RNA Production System (Promega). After RNA clean up (Qiagen RNeasy Mini kit), transfection was performed as previously described [26].

400 microliters of a Huh-7.5.1 or Huh7 cell suspension (10⁷ cells/ml) was placed in a 0.4-cm cuvette with 10 μg of Jc1-luc or HCV2a/2aChRluc RNA and electroporated (Bio-Rad Gene Pulser System) using a single square wave at 260 V and 25 ms pulse length. The cells were plated in 15-cm tissue culture dishes (Corning) and supernatant was harvested and concentrated using a centrifugal filter (Amicon 100K, Millipore).

Titration of infectious HCV. Naïve Huh-7.5.1 cells plated into a 96-well plate were infected with serial 10-fold dilutions of the infectious supernatants. The inoculum was incubated with cells overnight at 37° C. then replaced with fresh complete media and cultured for an additional 72 hours. The cells were stained using a monoclonal mouse anti-CORE IgG antibody (#MA1-080, Thermo Scientific) at a dilution of 1:300 followed by incubation with a 1:100 dilution of HRP-conjugated polyclonal goat anti-mouse IgG (#12-349, Millipore) for 1 hour at room temperature. The infectivity titer was calculated from the average number of foci counted under a microscope in the last and second last well of the dilution series that yielded CORE positive foci.

Luciferase compound activity assay. Cell culture and luciferase compound assays were performed as previously described. See Wyles et al., J. Virol. 2007, 81:3005-8; Wyles et al., Antimicrob Agents Chemother. 2009, 53:2660-2; Grünberger et al., J. Infect. Dis. 2008, 197:42-5; Wyles et al., Antimicrob Agents Chemother, 2008, 52:1862-4. Briefly, Huh7.5.1 cells were seeded into 96-well plates at a density of 10,000 cells per well in 100 μl medium. After incubating overnight for attachment, Jc1-luc virus at an MOI 0.01 was added to wells with or without compounds at the specified concentrations. All conditions were run in triplicate. After 24 hours, medium was aspirated and replaced with 100 μl of complete medium containing an identical concentration of compound(s) followed by additional 48 hour incubation. The luciferase assay (Bright-Glo; Promega) was carried out according to the manufacturer's instructions. Luciferase activity was determined using a microplate luminometer (Veritas microplate luminometer; Turner Biosystems). The relative light units (RLU) for each condition were reported as the mean±the standard error of the mean for the three wells.

Synergy testing. The 50% effective concentration (EC₅₀) of Compound 5 and other anti-HCV compounds was determined independently and used to set the range of concentrations for synergy experiments. Compound 5 was tested in combination with each of the anti-HCV compounds listed above at two twofold serial dilutions above and below the EC₅₀.

Compound 5 with ribavirin or VX950 in the HCV2a/2aChRluc system. 100 μl of serially diluted compound solutions (100, 30, 10, 3, 1, 0.3, and 0.1 nM) was inoculated onto Huh-7 cells in a 96-well plate. Subsequently, 100 ul (TCID50, ˜0.6) of HCV2a/2aChRluc virus was added onto the cells. After co-incubation for 72 hrs, Renilla luciferase expression was determined (Renilla Luciferase Assay, Promega) and reported as relative light units (RLU).

Data analysis. Determinations of compound interactions were quantified based on the median-effect principle described by Chou and Talalay. Chou T., Talalay P., Adv. Enzyme. Regul., 1984, 22:27-55. Combination indices (CIs) were determined using Calcusyn (Biosoft) for each experiment at the EC₅₀, EC₇₅, and EC₉₀ levels. Five replicates per condition were evaluated. A CI<0.9 was considered synergistic, a CI≧0.9 and ≦0.1 was considered additive, and a CI>1.1 was deemed antagonistic.

The EC₅₀ (±the standard error of the mean) for Compound 5 and each of the companion compounds, using the Jc1-Luc assay system, are listed in Table 4.

TABLE 4
Inhibitory activity and cytotocity of anti-HCV
compounds on Jc1-luc in vitro

| Compound | EC₅₀ (nM) | CC₅₀ (μM) |
|---|---|---|
| Compound 5 | 20.19 ± 1.37 | >100 |
| Interferon-alpha | 3.01 ± 0.39 IU/ml | >100 IU/ml |
| BILN2061 | 492.44 ± 56.69 | 35 |
| 2′-C-methyladenosine | 251.76 ± 33.54 | >100 |
| VX1 | 88.14 ± 12.73 | 100 |

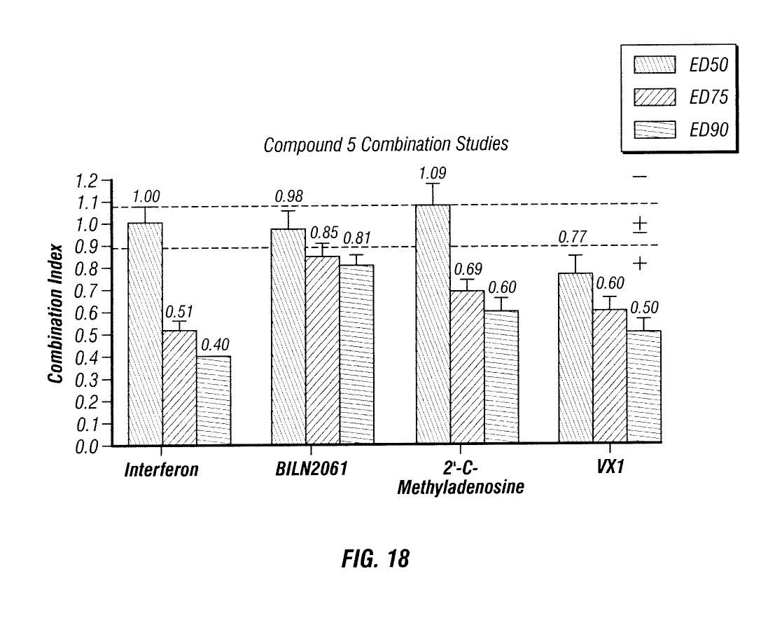

Synergy experiment results. Results for the synergy experiments are shown in FIG. 18 as an expression of the Combination Index (CI) of Compound 5 in combination with the anti-HCV compounds tested. Numerical values above the bars are the mean CI in FIG. 1. Error bars represent the standard error of the mean of the CI. The 2 lines at 0.9 and 1.1 represent the bounds of an additive interaction. +, synergy; ±, additivity; −, antagonism. ED₅₀, ED₇₅, and ED₉₀ refer to the combination index at the EC₅₀, EC₇₅, and EC₉₀, respectively, of each compound.

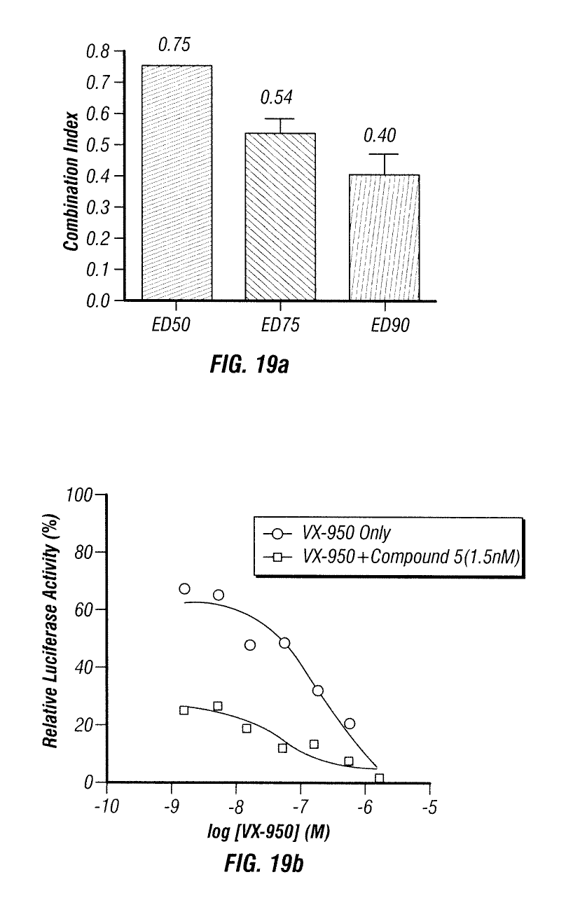

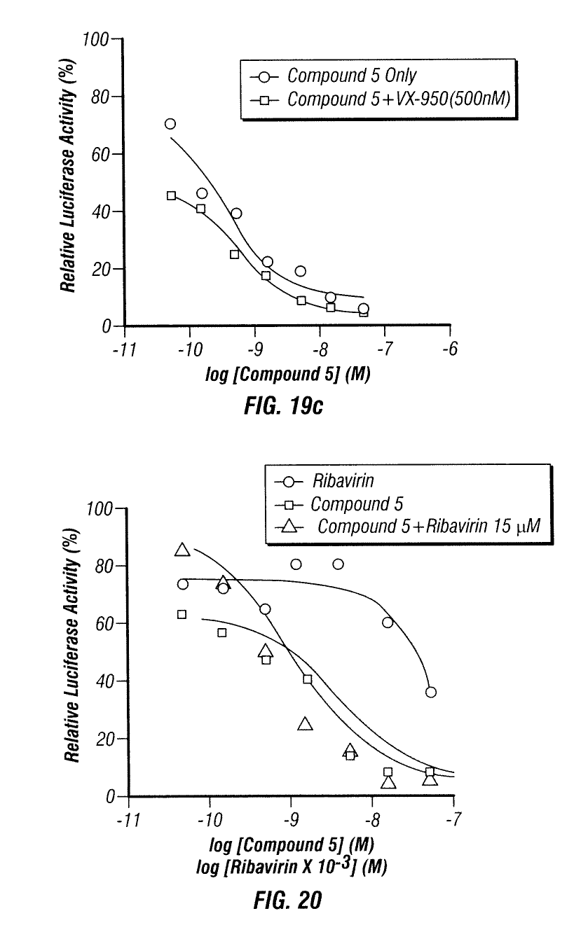

Compound 5 was not antagonistic with any compound studied. Specifically, Compound was additive with interferon-alpha, BILN2061 and 2′-C— methyladenosine at 50% effective dose (ED₅₀), the CI were 1.00, 0.98 and 1.09, respectively. At the 75% effective dose (ED₇₅) and 90% effective dose (ED₉₀), synergy was seen with all compounds tested. Combinations of Compound 5 and VX1 showed consistent synergy at ED₅₀, ED₇₅ and ED₉₀. Similar results were obtained in the HCV2a/2aCHRLuc system where Compound 5 also showed consistent synergy with VX-950. See FIG. 19. No compounds combinations showed cytotoxicity at the highest concentrations used in the synergy studies.

Compound 5 with ribavirin. Although ribavirin alone does not exhibit strong anti-viral activities in vitro, it is a key component of the standard of care in patients. Varying amounts of Compound 5 were co-incubated with a high concentration of ribavirin (15 μM), ribavirin alone did not have significant inhibitory effect on the infectivity of 2a chimeric virus. See FIG. 20. However, no antagonistic effect was discerned in the combination of Compound 5 and ribavirin.

### Example 13

### Resistance Study of Compound 5 and VX-950

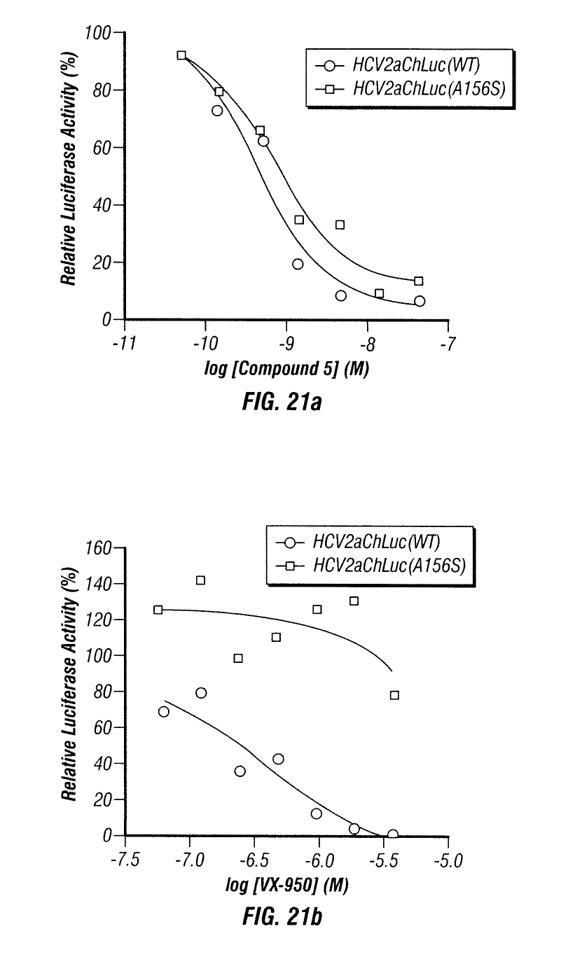

Compound 5 and VX-950 were evaluated for viral resistance in a NS3 protease mutant (A1565). Mutations in the alanine at position 156 of the NS3 coding region have been shown to confer resistance to many HCV protease inhibitors, such as SCH-503034 (boceprevir, Schering-Plough), SCH-6 (Schering-Plough), BILN-2061 (Boehringer Ingelheim), and VX-950 (Vertex). Therefore, the A156S mutation in the backbone of HCV2aChLuc genome to investigate the infectivity of the mutant virus in the presence of Compound 5 and VX-950. As expected, HCV2aChRLuc (A156S) showed high level resistance to VX-950 as compared to wild type HCV2aChRLuc. In contrast, no significant difference was observed in the inhibition of infection for both wild type and the mutant virus by Compound 5 indicating the expected lack of cross-resistance between a protease inhibitor and a viral entry inhibitor. See FIG. 21.

All publications and patent applications cited in this specification are herein incorporated by reference as if each individual publication or patent application were specifically and individually indicated to be incorporated by reference. While the invention has been described in terms of various preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method for the treatment of a hepatitis C virus infection in a host, comprising administering to a host infected with a hepatitis C virus an effective amount of a compound of formula I or II, a stereoisomer thereof, or a pharmaceutically acceptable salt thereof: 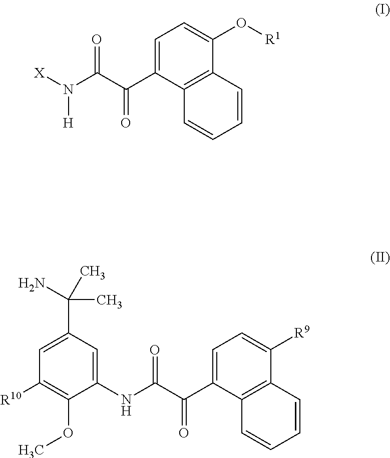
wherein 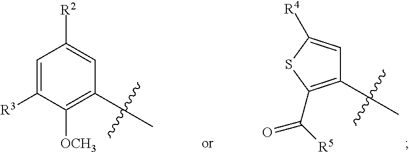
X is 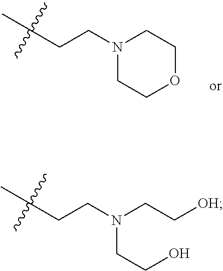
R¹ is methyl, ethyl, isopropyl,
R² is C₁-C₈ alkyl; C₅-C₈ cycloalkyl, or C₇-C₁₀ arylalkyl;
R³ is hydrogen, cyano, —CONHR⁶, —NHSO₂R⁷ or —SO₂N(R⁸)₂;
R⁴ is C₁-C₄ alkyl;
R⁵ is C₁-C₄ alkoxy or —N(R⁸)₂;
R⁶ is 2-pyridyl or C₁-C₆ alkyl, wherein one or more carbon atoms is optionally replaced by an oxygen atom;
R⁷ is C₁-C₄ alkyl, CH₂CF₃, benzyl or phenyl;
R⁸ is C₁-C₄ alkyl;
R⁹ is bromo or 6-(methylamino)pyridin-3-yl;
R¹⁰ is hydrogen or —CONHR¹¹; and
R¹¹ is hydrogen or C₁-C₄ alkyl;
provided that if R³ is —NHSO₂R⁷ and R⁷ is methyl, then R¹ is not methyl;
provided that if R¹⁰ is hydrogen, R⁹ is 6-(methylamino)pyridin-3-yl; and
provided that if R¹¹ is cyclopropyl, R⁹ is bromo.

2. The method of claim 1, further provided that if R² is methyl, then R³ is not —NHSO₂R⁹.

3. The method of claim 1, further provided that if R² is methyl and R³ is —NHSO₂R⁹, then R⁹ is not methyl.

4. The method of claim 1, further provided that if R³ is —NHSO₂R⁹, then R² is not methyl.

5. The method of claim 1, further provided that if R³ is —NHSO₂R⁹ and R⁹ is methyl, R² is not methyl.

6. The method of claim 1, wherein R¹ is methyl, ethyl, isopropyl or

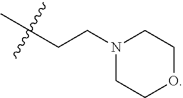

7. The method of claim 1, wherein R¹ is isopropyl or

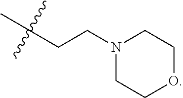

8. The method of claim 1, wherein R² is methyl, tert-butyl, cyclohexyl or 1-methyl-1-phenylethyl.

9. The method of claim 1, wherein R² is tert-butyl, cyclohexyl or 1-methyl-1-phenylethyl.

10. The method of claim 1, wherein R² is tert-butyl.

11. The method of claim 1, wherein R³ is hydrogen.

12. The method of claim 1, wherein R³ is —NHSO₂R⁹ and R⁹ is methyl.

13. The method of claim 1, wherein R⁴ is t-butyl.

14. The method of claim 1, wherein R⁵ is methoxy or dimethylamino;

15. The method of claim 1, wherein R⁶ is methyl, ethyl, propyl, methoxyethyl, methylenecyclopropyl or 2-pyridyl.

16. The method of claim 1, wherein R⁷ is methyl or ethyl.

17. The method of claim 1, wherein each R⁸ is methyl.

18. The method of claim 1, wherein R¹¹ is methyl or ethyl.

19. The method of claim 18, wherein R⁹ is 6-(methylamino)pyridin-3-yl.

20. The method of claim 1, wherein
R⁹ is bromo;
R¹⁰ is —CONHR¹¹; and
R¹¹ is C₁-C₄ alkyl.

21. The method of claim 1, wherein the compound, or a pharmaceutically acceptable salt thereof, has the formula:

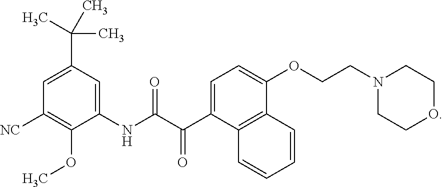

22. The method of claim 1, wherein the compound, or a pharmaceutically acceptable salt thereof, has the formula:

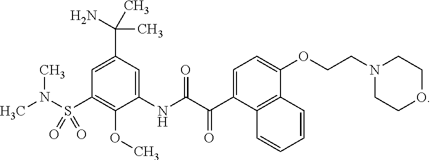

23. The method of claim 1, wherein the compound, or a pharmaceutically acceptable salt thereof, has the formula:

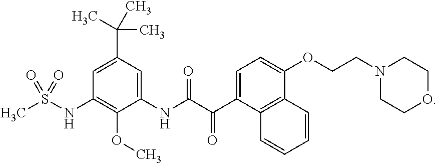

24. The method of claim 1, wherein the compound, or a pharmaceutically acceptable salt thereof, has the formula:

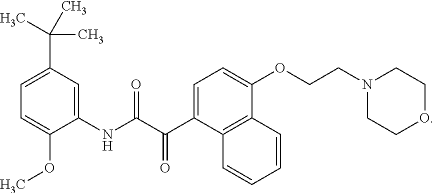

25. The method of claim 1, wherein the compound, or a pharmaceutically acceptable salt thereof, has the formula:

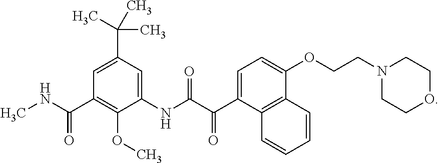

26. The method of claim 1, wherein the compound, or a pharmaceutically acceptable salt thereof, has the formula:

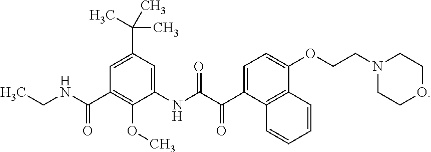

27. The method of claim 1, wherein the compound, or a pharmaceutically acceptable salt thereof, has the formula:

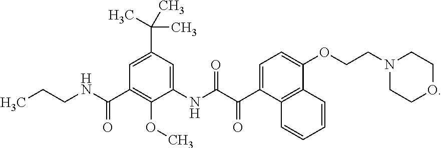

28. The method of claim 1, wherein the compound, or a pharmaceutically acceptable salt thereof, has the formula:

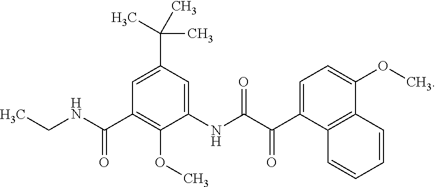

29. The method of claim 1, wherein the compound, or a pharmaceutically acceptable salt thereof, has the formula:

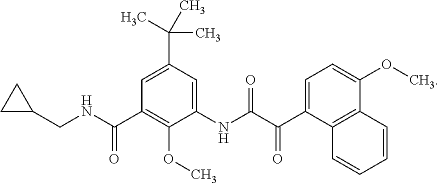

30. The method of claim 1, wherein the compound, or a pharmaceutically acceptable salt thereof, has the formula:

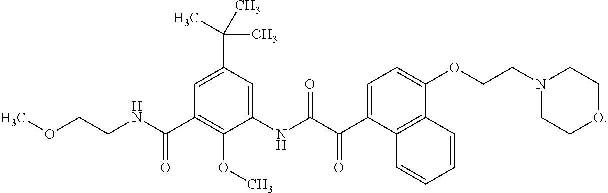

31. The method of claim 1, wherein the compound, or a pharmaceutically acceptable salt thereof, has the formula:

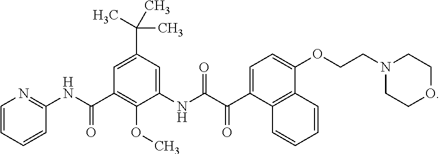

32. The method of claim 1, wherein the compound, or a pharmaceutically acceptable salt thereof, has the formula:

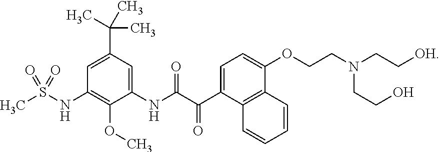

33. The method of claim 1, wherein the compound, or a pharmaceutically acceptable salt thereof, has the formula:

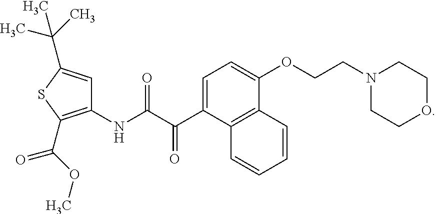

34. The method of claim 1, wherein the compound, or a pharmaceutically acceptable salt thereof, has the formula:

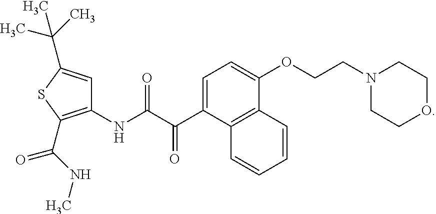

35. The method of claim 1, wherein the compound, or a pharmaceutically acceptable salt thereof, has the formula:

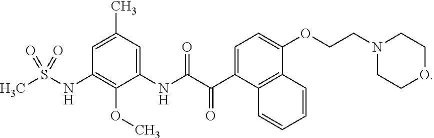

36. The method of claim 1, wherein the compound, or a pharmaceutically acceptable salt thereof, has the formula:

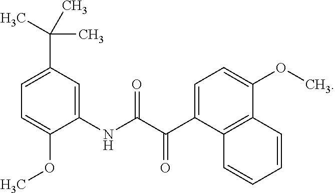

37. The method of claim 1, wherein the compound, or a pharmaceutically acceptable salt thereof, has the formula:

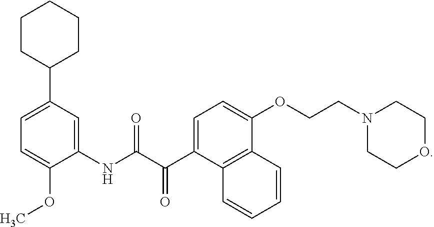

38. The method of claim 1, wherein the compound, or a pharmaceutically acceptable salt thereof, has the formula:

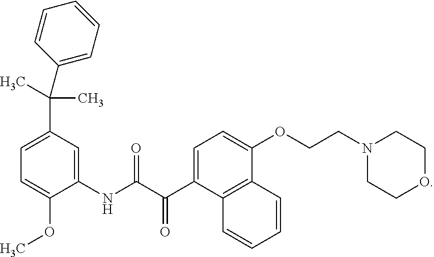

39. The method of claim 1, wherein the compound, or a pharmaceutically acceptable salt thereof, has the formula:

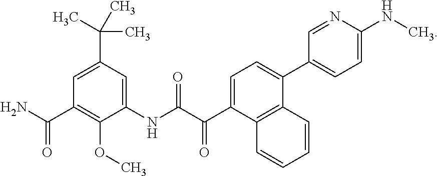

40. The method of claim 1, wherein the compound, or a pharmaceutically acceptable salt thereof, has the formula:

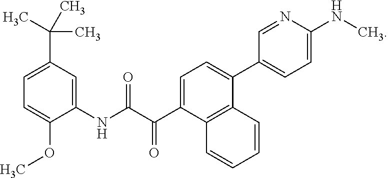

41. The method of claim 1, wherein the compound, or a pharmaceutically acceptable salt thereof, has the formula:

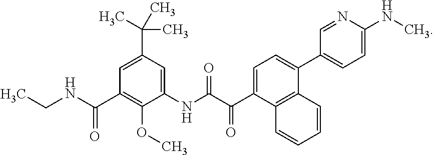

42. The method of claim 1, wherein the compound, or a pharmaceutically acceptable salt thereof, has the formula:

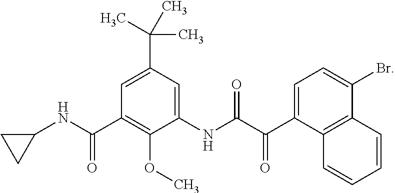

43. The method of claim 1, wherein the compound, or a pharmaceutically acceptable salt thereof, has the formula:

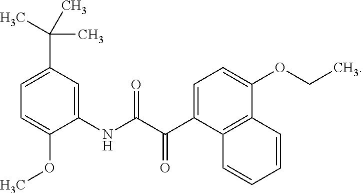

44. The method of claim 1, wherein the compound, or a pharmaceutically acceptable salt thereof, has the formula:

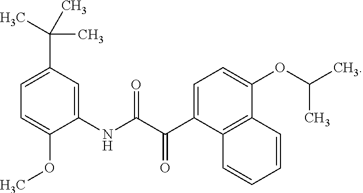

45. A method for the treatment of a hepatitis C virus infection in a host, comprising administering to a host infected with a hepatitis C virus an effective amount of a compound of formula I or II of claim 1, or a stereoisomer thereof, or a pharmaceutically acceptable salt thereof, in combination or alternation with one or more antiviral agents selected from the group consisting of a nucleoside polymerase inhibitor, a non-nucleoside polymerase inhibitor, a protease inhibitor, a cyclophilin modulator, an interferon and ribavirin.

46. The method of claim 45, wherein the compound of formula I or II, or a stereoisomer thereof, or a pharmaceutically acceptable salt thereof, is administered in combination or alternation with an interferon.

47. The method of claim 45, wherein the compound of formula I or II, or a stereoisomer thereof, or a pharmaceutically acceptable salt thereof, is administered in combination or alternation with an interferon and ribavirin.

48. A compound of formula I or II of claim 1, or a stereoisomer thereof, or a pharmaceutically acceptable salt thereof, for use in the treatment or prevention of a hepatitis C virus infection.

49. Use of a of formula I or II of claim 1, or a stereoisomer thereof, or a pharmaceutically acceptable salt thereof, in the manufacture of a medicament for the treatment or prevention of a hepatitis C virus infection.
